(19) United States
(12) Patent Application Publication
(10) Pub. No.: US 2024/0358856 A1
(43) Pub. Date: Oct. 31, 2024

(54) ADENO-ASSOCIATED VECTORS AND VIRIONS TO TREAT GALACTOSEMIA AND METHODS OF USE AND MANUFACTURE

(71) Applicant: Jaguar Gene Therapy, LLC, Lake Forest, IL (US)

(72) Inventor: Michael Hughes, London (GB)

(21) Appl. No.: 18/687,808

(22) PCT Filed: Sep. 1, 2022
(86) PCT No.: PCT/US2022/075871

§ 371 Date: Feb. 28, 2024

Related U.S. Application Data
(60) Provisional application No. 63/239,650, filed on Sep. 1, 2021.

(51) Int. Cl. A61K 48/00; A61K 38/45; C12N 15/86

(52) U.S. Cl. CPC A61K 48/005; A61K 38/45; A61K 48/0075; C12N 15/86; C12Y 207/07012; C12N 2750/14143; C12N 2830/48; C12N 2830/50

(57) ABSTRACT

Provided are recombinant adeno-associated virus (rAAV) vectors comprising a transgene to express galactose-1-phosphate uridylyl transferase (GALT); virions comprising said vectors (rAAV virions); methods of their production; methods of their use, including methods for treating galactosemia, GALT-deficiency, symptoms therefrom; and kits.

### FIELD OF THE INVENTION

Provided are viral constructs, particles and compositions for use in the treatment of galactose-1-phosphate uridylyltransferase (GALT) deficiency.

### BACKGROUND

Galactosemia is the inability to metabolize galactose, and type 1 galactosemia is specifically caused by polymorphisms in the enzyme galactose-1-phosphate uridylyltransferase (GALT). Accordingly, type 1 galactosemia can also be known as galactose-1-phosphate uridylyltransferase deficiency. GALT performs the second step of the Leloir pathway of galactose metabolism. In that step, GALT interconverts uridine diphosphate-glucose and galactose-1-phosphate into glucose-1-phosphate and uridine diphosphate-galactose in a reversible manner.

Type 1 galactosemia can be categorized into two categories. The first is classic galactosemia, and it occurs when the individual has a polymorphism resulting in lost translation of GALT or a complete or near complete (˜5% residual activity) loss of GALT activity, Classic galactosemia is an autosomal recessive inheritance. The second category of type 1 galactosemia is Duarte galactosemia, which involves a GALT with diminished activity, specifically around 25% of the normal levels of GALT activity of an individual having normal alleles. Examples of mutations causing type 1 galactosemia, a substitution from A to G in exon 6 of the GALT gene changes glutamine 188 to an arginine, and a mutation from A to G in exon 10, which changes asparagine 314 to an aspartic acid.

Required are gene therapies to introduce GALT to an individual suffering from type 1 galactosemia, GALT-deficiency, or failure to metabolize galactose.

### SUMMARY OF INVENTION

Provided are nucleic acid vector constructs for production of recombinant adeno-associated virus (AAV) virions encoding GALT. The vector constructs are used to generate recombinant AAV virions or particles which are administered for rAAV gene therapy in a subject suffering from galactosemia to deliver to cells of the subject a nucleic acid encoding GALT, thereby providing increased GALT activity to the subject to treat and ameliorate disease.

Provided are AAV vectors comprising an expression cassette which comprises a nucleotide sequence encoding human galactose-1-phosphate uridylyl transferase (hGALT), operably linked to one or more regulatory elements that promote expression of the hGALT coding sequence and a polyadenylation (poly(A)) tail signal; the promoter comprising a cytomegalovirus (CMV) early enhancer/chicken β-actin/rabbit B-globin splice acceptor (CAG) promoter or an elongation factor-1 (EF-1) promoter; the poly(A) tail signal comprising a bovine growth hormone (bGH) poly(A) tail signal or a simian virus 40 (SV40) poly(A) tail signal, flanked by inverted terminal repeat (ITR) nucleotide sequences. The hGALT may have the amino acid sequence of SEQ ID NO: 1 and may be encoded by a nucleic comprising a nucleotide sequence having at least 85% identity to the nucleotide sequence of SEQ ID NO.: 2 or a sequence reverse complement thereof, which encodes an hGALT, and, may comprise the nucleotide sequence of SEQ ID NO: 2. The vector may also contain a WPRE element, which may be located between the hGALT coding sequence and the polyA signal sequence. The ITR sequences may be wild type sequences, such as AAV2 ITRs or may include a modified ITR sequence that results in a double-stranded “self complementary” AAV vector.

Particular expression cassettes are provided and have nucleotide sequences of SEQ ID NO.: 3 (pscAAV-CAG-hGALT), SEQ ID NO.: 4 (pAAV2ITR-CAG-hGALT), or SEQ ID NO.: 5 (pAAV2ITR-EF1a-hGALT) (or a sequence reverse complementary thereto), or may be at least 85% identical to the nucleotide sequence of SEQ ID NO.: 3 (pscAAV-CAG-hGALT), SEQ ID NO.: 4 (pAAV2ITR-CAG-hGALT), or SEQ ID NO.: 5 (pAAV2ITR-EF1a-hGALT), and encodes a human GALT.

Particular expression cassettes (not including any flanking ITRs sequences) are provided and have nucleotide sequences of SEQ ID NO.: 19 (pscAAV CAG-hGALT), SEQ ID NO.: 20 (pAAV2ITR-CAG-hGALT), or SEQ ID NO.: 21 (pAAV2ITR EF1a-hGALT) (or a sequence reverse complementary thereto), or may be at least 85% identical to the nucleotide sequence of SEQ ID NO.: 19 (pscAAV-CAG-hGALT), SEQ ID NO.: 20 (pAAV2ITR-CAG-hGALT), or SEQ ID NO.: 21 (pAAV2ITR-EF1a-hGALT), and encodes a human GALT.

Also provided are plasmids comprising these expression cassettes, including the plasmid pAAV2ITR-CAG-hGALT-KanR, depicted in FIG. 1, the nucleotide sequence of which is provided in FIG. 2 (also the nucleotide sequence of SEQ ID NO: 7 and the reverse complement thereof); the plasmid pAAV2ITR-EF1a-hGALT-KanR, depicted in FIG. 3, the nucleotide sequence of which is provided in FIG. 4 (also nucleotide sequence of SEQ ID NO: 8) and pscAAV-CAG-hGALT-KanR, depicted in FIG. 5, the nucleotide sequence of which is provided in FIG. 6 (also the nucleotide sequence of SEQ ID NO: 6).

Also provided are recombinant AAV virions comprising a recombinant AAV genome (an expression cassette) described herein encoding hGALT (for example, the expression cassettes of SEQ ID NO: 3, 4 or 5) and an AAV capsid. The AAV capsid may be an AAV9 capsid (amino acid sequence SEQ ID NO: 18).

Also provided are methods of treating galactosemia or in increasing galactose metabolism in a subject in need thereof, particularly, a human subject. Also included are methods of reducing a disease condition in a subject suffering from galactosemia by administering rAAV virions described herein, wherein the disease condition comprises jaundice, hepatosplenomegaly, hepatocellular insufficiency, hypoglycemia, renal tubular dysfunction, muscle hypotonia, sepsis, cataract, ataxia, tremor, decreased bone density, or primary ovarian insufficiency. Provided are pharmaceutical compositions and methods of administration, including, but not limited to, intravenous administration or intrathecal administration.

Also provided are host cells for and methods of producing the recombinant AAV virions as described herein.

### EMBODIMENTS

1. A recombinant adeno-associated virus (AAV) vector comprising an expression cassette, which comprises a nucleotide sequence encoding human galactose-1-phosphate uridylyl transferase (hGALT), operably linked to one or more regulatory elements that promote expression of the hGALT coding sequence and a polyadenylation (poly(A)) tail signal; the promoter comprising a cytomegalovirus (CMV) early enhancer/chicken β-actin/rabbit β-globin splice acceptor (CAG) promoter or an elongation factor-1 (EF-1) promoter; the poly(A) tail signal comprising a bovine growth hormone (bGH) poly(A) tail signal or a simian virus 40 (SV40) poly(A) tail signal, flanked by inverted terminal repeat (ITR) nucleotide sequences.

2. The recombinant AAV vector of embodiment 1, the hGALT comprising the amino acid sequence of SEQ ID NO.: 1.

3. The recombinant AAV vector of embodiment 1 or embodiment 2, the nucleic acid that encodes GALT comprising a nucleic acid having at least 85% identity to the nucleotide sequence of SEQ ID NO.: 2 or a sequence reverse complementary thereto.

4. The recombinant AAV vector of any one of embodiments 1-3, the nucleic acid that encodes GALT comprising or consisting of the nucleotide sequence of SEQ ID NO.: 2 or a sequence reverse complementary thereto.

5. The recombinant AAV vector of any one of embodiments 1-4 wherein the promoter has a nucleotide sequence of SEQ ID NO: 9 or SEQ ID NO: 10, or the reverse complement thereof, and the polyA signal sequence has a nucleotide sequence of SEQ ID NO: 15 or SEQ ID NO: 16, or the reverse complement thereof.

6. The recombinant AAV vector of any one of embodiments 1-5, which comprises a WPRE element.

7. The recombinant AAV vector of any one of embodiments 1-6, wherein the ITRs comprise a 5′ AAV2 ITR having a nucleotide sequence of SEQ ID NO: 11 and a 3′ AAV2 ITR having a nucleotide sequence of SEQ ID NO: 12, or the reverse complement thereof.

8. The recombinant AAV vector of any one of embodiments 1-6 wherein the ITRs comprise a 5′ ITR having a nucleotide sequence of SEQ ID NO: 11 and a modified self-complementary 3′ ITR having a nucleotide sequence of SEQ ID NO: 13, or reverse complement thereof.

9. The recombinant AAV vector of any one of embodiments 1-8 comprising an expression cassette comprising a nucleic acid that has at least 85% identity to the nucleotide sequence of SEQ ID NO.: 3, SEQ ID NO.: 4, or SEQ ID NO.: 5, or a sequence reverse complementary thereto, and encodes a human GALT.

10. The recombinant AAV vector of any one of embodiments 1-8 comprising an expression cassette comprising a nucleic acid that has at least 85% identity to the nucleotide sequence of SEQ ID NO.: 19, SEQ ID NO.: 20, or SEQ ID NO.: 21, or a sequence reverse complementary thereto, and encodes a human GALT.

11. The recombinant AAV vector of any one of embodiments 1-9 comprising an expression cassette comprising a nucleic acid having a nucleotide sequence of SEQ ID NO.: 3, SEQ ID NO.: 4, or SEQ ID NO.: 5, or a sequence reverse complementary thereto.

12. The recombinant AAV vector of any one of embodiments 1-8 or embodiment 10 comprising an expression cassette comprising a nucleic acid having a nucleotide sequence of SEQ ID NO.: 19, SEQ ID NO.: 20, or SEQ ID NO.: 21, or a sequence reverse complementary thereto.

13. A recombinant self-complementary AAV (scAAV) vector comprising the recombinant AAV vector of any one of embodiments 1-12 comprising an scAAV ITR.

14. A recombinant AAV virion comprising: 1) an AAV capsid; and 2) the recombinant AAV vector of any one of embodiments 1-13; and the AAV capsid protein encapsulating the recombinant AAV vector.

15. The AAV virion of embodiment 14, wherein the AAV capsid has an amino acid sequence at least 85% identical to SEQ ID NO: 18 (AAV9).

16. The AAV virion of embodiment 15, wherein the AAV capsid has an amino acid sequence of SEQ ID NO: 18.

17. A method for treating galactosemia in a subject in need thereof, the method comprising administering to the subject a therapeutically effective amount of the AAV virion of any one of embodiments 14-16.

18. A method of increasing galactose metabolism in a subject in need thereof, the method comprising administering to the subject a therapeutically effective amount of the AAV virion of any one of embodiments 14-16.

19. A method of reducing a disease condition in a subject suffering from galactosemia, the method comprising administering to the subject a therapeutically effective amount of the AAV virion of any one of embodiments 14-16, the disease condition comprising jaundice, hepatosplenomegaly, hepatocellular insufficiency, hypoglycemia, renal tubular dysfunction, muscle hypotonia, sepsis, cataract, ataxia, tremor, decreased bone density, or primary ovarian insufficiency.

20. The method of any one of embodiments 17-19, said administering comprising intravenous administration, intra-arterial, intramuscular administration, intracardiac administration, intrathecal administration, subventricular administration, epidural administration, intracerebral administration, intracerebroventricular administration, sub-retinal administration, intravitreal administration, intraarticular administration, intraocular administration, intraperitoneal administration, intrauterine administration, intradermal administration, subcutaneous administration, transdermal administration, transmucosal administration, or administration by inhalation.

21. The method of any one of embodiments 17-19, the administering comprising intravenous administration, or intrathecal administration.

22. An AAV vector plasmid comprising 1) an origin of replication and 2) the recombinant AAV vector of any one of embodiments 1-13.

23. The AAV vector plasmid of embodiment 22, comprising a nucleic acid having at least 85% identity to the nucleotide sequence of SEQ ID NO.: 6, SEQ ID NO.: 7 or SEQ ID NO.: 8, which encodes a human GALT.

24. The AAV vector plasmid of embodiment 22, comprising a nucleic acid having at least 85% identity to the nucleotide sequence of SEQ ID NO.: 19, SEQ ID NO.: 20 or SEQ ID NO.: 21, which encodes a human GALT.

25. The AAV vector plasmid of embodiment 22 or 23 comprising the nucleotide sequence of SEQ ID NO.: 6, SEQ ID NO.: 7, or SEQ ID NO.: 8.

26. The AAV vector plasmid of embodiment 22 or 24 comprising the nucleotide sequence of SEQ ID NO.: 19, SEQ ID NO.: 20, or SEQ ID NO.: 21.

27. A cell comprising an AAV vector plasmid of any one of embodiments 22-26 and a second plasmid comprising nucleotide sequences encoding rep and cap; the cap encoding a VP1, a VP2, and a VP3; the rep encoding rep78, rep68, rep 52, and rep 40.

28. The cell of embodiment 27, the cap being AAV9 cap.

29. A method of producing an AAV virion, the method comprising culturing a host cell comprising the AAV vector plasmid of any one of embodiments 22-26, a second plasmid encoding the cap and rep; the cap encoding the VP1, the VP2, and the VP3; the rep encoding rep78, rep68, rep 52, and rep 40; and any additional adenoviral helper functions, under conditions sufficient to produce the AAV virion; and isolating the AAV virion produced by the host cell.

### BRIEF DESCRIPTION OF THE DRAWINGS

FIG. 1 depicts a map of the plasmid pAAV2ITR-CAG-hGALT-KanR, which comprises two AAV 2 inverted-terminal repeats (ITR) (SEQ ID NO.: 11 and 12) flanking an expressing region comprising a cytomegalovirus (CMV) early enhancer/chicken β-actin/rabbit β-globin splice acceptor (CAG) promoter (SEQ ID NO.: 9), a nucleic acid encoding human GALT (hGALT) (SEQ ID NO.: 2), a woodchuck hepatitis virus post-transcriptional regulatory element (WPRE) (SEQ ID NO.: 14), and a bovine growth hormone poly adenylation (bGH poly(A)) signal sequence (SEQ ID NO.: 15). The plasmid further comprises an ampicillin resistance gene (AmpR) promoter controlling expression of a kanamycin resistance gene sequence (KanR), a CMV origin of replication (ori), and catabolite activator protein (CAP) binding site, a lactose (lac) promoter controlling a lac operator, and an M13 reverse primer region.

FIG. 2 provides the double stranded nucleotide sequence of the pAAV2ITR-CAG-hGALT-KanR plasmid (SEQ ID NOs.: 7), which includes the nucleotide sequence encoding the hGALT (SEQ ID NO.: 2) and the amino acid sequence of the hGALT (SEQ ID NO.: 1).

FIG. 3 depicts a map of the plasmid pAAV2ITR-EF1-hGALT-KanR, which comprises two AAV 2 ITRs flanking an expressing region comprising a elongation factor-1α (EF-1α) promoter (SEQ ID NO.: 10), a nucleic acid encoding hGALT, WPRE, and bGH poly(A) signal sequence. The plasmid further comprises an AmpR promoter controlling expression of a KanR gene sequence, a CMV origin of replication (ori), and CAP binding site, a lac promoter controlling a lac operator, and an M13 reverse primer region.

FIG. 4 provides the double stranded nucleotide sequence of pAAV2ITR-EF1-hGALT-KanR plasmid (SEQ ID NOs.: 8), which includes the nucleotide sequence encoding the hGALT (SEQ ID NO.: 2) and the amino acid sequence of the hGALT (SEQ ID NO.: 1).

FIG. 5 depicts a map of the plasmid pscAAV-CAG-hGALT-KanR, which comprises one AAV 2 ITR upstream of an expressing region comprising the CAG promoter, a nucleic acid sequence encoding hGALT, WPRE, an simian virus 40 (SV40) poly(A) signal sequence (SEQ ID NO.: 16), and a self-complementary AAV (scAAV) ITR (SEQ ID NO.: 13). The plasmid further comprises an AmpR promoter controlling expression of a KanR gene sequence, a CMV origin of replication (ori), and CAP binding site, a lac promoter controlling a lac operator, and an M13 reverse primer region.

FIG. 6 provides the double stranded nucleotide sequence of pscAAV-CAG-hGALT-KanR plasmid (SEQ ID NOs.: 6), which includes the nucleotide sequence encoding the hGALT (SEQ ID NO.: 2) and the amino acid sequence of the hGALT (SEQ ID NO.: 1).

### DETAILED DESCRIPTION

### Definitions

When interpreting the description and claims, any one of “comprising,” “consisting of,” “consisting essentially of,” “is selected from the group consisting of,” “is at least selected from the group consisting of,” “is at least one selected from the group consisting of,” and “is, “being,” and “are,” or an equivalent thereof should be understood to contemplate any other and provide support for replacement of that which was recited with any other. For example, when “comprising A, B, or C” is recited, contemplated therein is replacing such, and provides support for embodiments which replace, with: “is A, B, or C,” “consists of A, B, and C,” “consists essentially of A, B, or C.” “is at least selected from the group consisting of A, B, and C,” “is at least one selected from the group consisting of A, B, and C,” or any equivalent thereof.

Further, recitation of “or” contemplates and supports, “one or more of,” “one or a combination of,” or “and,” as in “and/or.” For example, “A, B, or C” contemplates and supports embodiments with: A alone; B alone; C alone; the combination of A and B; the combination of A and C; the combination of B and C; and the combination of A, B, and C. Further to which, within recitation of “closed” language (e.g. consisting of), as well as within recitation of “open” language (e.g. comprising), the recitation of a list, as in “A, B, or C.” contemplates one or a combination within that list, unless otherwise specified. For example, “consisting of A, B, or C” contemplates and supports embodiments with: A alone; B alone; C alone; the combination of A and B; the combination of A and C; the combination of B and C; and the combination of A, B, and C. Recitation of “and/or” contemplates and supports not only the combination of all within the list (i.e. “A, B, and/or C” contemplates “A, B, and C”), but also “one or more of” or “one or a combination of.” For example “A, B, and C” contemplates: A alone; B alone; C alone; the combination of A, B and C; the combination of A and B; the combination of A and C; and the combination of B and C.

The recitation of a list of alternatives with an “and,” as in for example “selected from the group consisting of,” contemplates and provides support for combinations within that list, unless otherwise stated. For example, “is selected from the group consisting of A, B, and C” is to be understood to contemplate and support “is selected from the group consisting of A, B, C, and combinations thereof” and to be coextensive with “is at least one selected from the group consisting of A, B, and C” or such that “group” includes: A alone, B alone, C alone, A and B in combination, A and C in combination, B and C in combination, and A, B, and C in combination.

“Is at least selected from the group consisting of A, B, and C” is contemplated to include embodiments and supports embodiments wherein what is after “is” is open due to recitation of “at least” such it is coextensive with “comprises a member of the group consisting of A, B, and C” or such that “consisting of” modifies the meaning of “group” alone and not “is selected from the group.”

Further, recitation of a component in an embodiment also contemplates and supports exclusion, explicitly, of said component from the embodiment. For example, “comprising A, B, or C” supports embodiments, which comprise A or B, but specifically exclude C.

The articles “a” and “an” are used herein to refer to one or to more than one (i.e., to at least one) of the grammatical object of the article. By way of example, “an element” means one element or more than one element. For example, “comprising an A, a B, or a C” contemplates and supports embodiments comprising two or more A, two or more B, and two or more C.

Unless defined otherwise, all technical and scientific terms have the same meaning as commonly understood by one of ordinary skill in the art to which the embodiments pertain. The preferred materials and methods are described, but it is understood that any methods and materials similar or equivalent to those described can be used in the practice of embodiments. The terminology used herein is for the purpose of describing particular embodiments only and is not intended to be limiting. In describing and claiming the present invention, the following terminology will be used.

“About” as used herein when referring to a measurable value such as an amount, a temporal duration, and the like, is meant to encompass variations of .+−0.20% or .+−0.10%, more preferably .+−0.5%, even more preferably .+−0.1%, and still more preferably .+−0.1% from the specified value, as such variations are appropriate to perform the embodiments.

As used herein, the term “cell line” refers to a population of cells capable of continuous or prolonged growth and division in vitro. Often, cell lines are clonal populations derived from a single progenitor cell. Spontaneous or induced changes can occur in karyotype during storage or transfer of such clonal populations. Therefore, cells derived from the cell line referred to may not be precisely identical to the ancestral cells or cultures, and the cell line referred to includes such variants.

The term “expression” as used herein is defined as the transcription and/or translation of a particular nucleotide sequence driven by its promoter.

As used herein, the term “host cell” refers to any cell that harbors, or is capable of harboring, a substance of interest. Often a host cell can be a mammalian cell (e.g., a non-human primate, rodent, or human cell). In some aspects, the host cell can be a mammalian cell, a yeast cell, a bacterial cell, an insect cell, a plant cell, or a fungal cell. A host cell can be used as a recipient of an AAV helper construct, an AAV plasmid encoding a recombinant AAV genome comprising a transgene, an accessory function vector, or other transfer DNA associated with the production of recombinant AAV (rAAV) virions. The term includes the progeny of the original cell which has been transfected. Thus, a “host cell” as used herein can refer to a cell which has been transfected with an exogenous DNA sequence. It is understood that the progeny of a single parental cell may not necessarily be completely identical in morphology or in genomic or total DNA complement as the original parent, due to natural, accidental, or deliberate mutation.

“Identity” as used herein refers to the subunit sequence identity between two polymeric molecules particularly between two amino acid molecules, such as, between two polypeptide molecules or two nucleic acid molecule, such as polynucleotides. When two amino acid sequences have the same residues at the same positions; e.g., if a position in each of two polypeptide molecules is occupied by an arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical. In the case of an insertion or deletion, identity is understood to realign those thereafter which would be identical and is considered to be not identical at the insertion or deletion.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

By the term “modified” as used herein, is meant a changed state or structure of a molecule or cell of the invention. Molecules may be modified in many ways, including chemically, structurally, and functionally. Cells may be modified through the introduction of nucleic acids.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

A “nucleic acid,” as used herein, is interchangeable with “polynucleotide” or “a specific sequence of nucleotide.” These terms refer to a discrete sequence that performs a specific function directly or indirectly in a cell. That function includes encoding a sequence of a gene that is transcribed into mRNA and translated into protein and regulating said transcription (i.e. as a promoter would) and/or translation (i.e. as microRNA would). A nucleic acid inherently has a sequence. Thereby, “a nucleic acid comprising SEQ ID NO.: X” can be used to contemplate and support “a nucleic acid comprising the sequence of SEQ ID NO.: X.” In recombinant molecular biology, discrete nucleic acids can be combined. In some embodiments, a nucleic acid that encodes a protein can be ligated to a promoter (which is a nucleic acid), and a cis-acting element of a viral vector (i.e. an inverted-terminal repeat (ITR), which is also a nucleic acid). For convenience, a “nucleic acid” might be used to refer to the discrete elements within the larger nucleic acid, which could be referred to as “a polynucleotide,” “an expression region” (i.e. a polynucleotide comprising a promoter and a nucleic acid that encodes a protein), or “a vector” (see definition below).

“Encoding” refers to the inherent property of a nucleic acid to serve as a template, whether directly (i.e. a sense strand) or indirectly (i.e. an antisense strand) for synthesis of peptide, polypeptides, proteins, or other nucleic acids (i.e. rRNA, tRNA, microRNA). A nucleic acid can “encode” whether it is the sense strand, antisense strand, or a double-stranded segment thereof. The sense strand directly encodes the rRNA, tRNA, microRNA, or mRNA. The mRNA then serves as the template for translation of a peptide, polypeptide, or protein. The anti-sense strand is generally considered to be the reverse complementary sequence and is sometimes called a “non-coding” strand in the art (although for present purposes “non-coding” is a misnomer because the non-coding strand still “encodes” the genetic information by perpetuating it during semi-conservative replication by acting as a template for the polymerization of a new, sense strand). Within semi-conservative replication two single strands in double-stranded nucleic acids are separated, and a new strand is polymerized from the information from each of the single-stranded nucleic acids (i.e. single-stranded template), regardless of whether one single-stranded template is the sense strand (e.g. that which is used to transcribe mRNA and thereby, or directly, encode the translate or protein) or the antisense strand. By perpetuating the genetic information, the antisense strand is still encoding the genetic information for, for example, a protein. Accordingly, “a nucleic acid encoding X”, includes sense and antisense sequences or strands whether X is a peptide, a polypeptide, or a protein or X is a sequence that encodes a rRNA, tRNA, microRNA, antisenseRNA, etc.

Further to which, “nucleic acid encoding X,” includes RNA, DNA, and combinations thereof, since nucleic acids are synthesized from transcription, reverse-transcription, and replication, as naturally occurring processes and man-made processes (recombinant biology, molecular biology, etc).

Accordingly, a recited nucleic acid sequence contemplates and supports the complementary version thereof, the reverse complementary version thereof, and double-stranded versions thereof. That is, “a nucleic acid comprising SEQ ID NO.: X” is to be understood, contemplate, and support “a nucleic acid comprising the reverse complementary of SEQ ID NO.: X” or, using the nomenclature regarding the prime symbol as in “′”, “a nucleic acid comprising SEQ ID NO.: X′.” unless otherwise specified. For example, “the nucleic acid comprising SEQ ID NO.: X” wherein SEQ ID NO.: X is 5′-ATGCC-3′ contemplates and supports the reverse complementary of SEQ ID NO.: X, and specifically 5′-GGCAT-3.

As noted above, a recited nucleic acid sequence contemplates and supports conversion between RNA and DNA versions thereof. For example, if SEQ ID NO.: X is “5′-ATGCC-3′,” contemplated and supported is 5′-AUGCC-3′, as well as the reverse complementary thereof, 5′-GGCAU-3.

With regard to an AAV vector or an AAV virion, the above-noted incorporation of reverse complementary sequences and double-stranded segments into the definition of “a nucleic acid” and the above-noted use of “encoding” as including sense and antisense strands, is intended to incorporate the means by which the AAV vector can introduce an exogenous nucleic acid sequence that encodes nucleic acid or a protein into the cell. It is further intended to incorporate, in some embodiments, processes whereby said introduction results in the expression of said nucleic acid (i.e. miRNA or antisense RNA) or protein (i.e. galactose-1-phosphate uridylyltransferase (GALT)).

Take for example, a nucleic acid encoding a protein, and an AAV vector comprising a nucleic acid encoding said protein. When a typical (i.e. naturally occurring) AAV vector encoding one sense or one antisense strand of the nucleic acid that encodes said protein enters the cell, the inverted-terminal repeats (ITRs) prime the synthesis of a sequence reverse complementary to the sense strand or antisense strand of the nucleic acid that encodes said protein. The polymerization thereby forms a segment of double-stranded DNA comprising the sense and antisense strands, regardless of whether the sense version or antisense version was first introduced to the cell. In this regard, the entire nucleic acid including ITRs and sense and antisense nucleic acids encoding a protein can be one single-stranded DNA, which loops upon itself to form a double-stranded segment, wherein the base-pairs the sense and antisense nucleic acids encoding the protein align.

From this segment of double-stranded DNA, transcription of mRNA and translation of said protein is achieved from said sense strand of DNA, regardless of whether the AAV vector comprised only the sense strand or only the antisense strand when first entering the cell. In this regard, “an AAV vector comprising a nucleic acid encoding protein X” includes, contemplates, and supports embodiments in which the nucleic acid is the sense strand encoding protein X, the antisense strand encoding protein X, a double-stranded nucleic acid encoding protein X, and a single stranded nucleic acid comprising sense and antisense strands wherein the sense and antisense strands form a segment of double-stranded nucleic acid.

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence.

The term “promoter” as used herein is defined as a DNA sequence recognized by the synthetic machinery of the cell, or introduced synthetic machinery, required to initiate the specific transcription of a polynucleotide sequence. In some instances, this sequence may be the core promoter and in other instances, this sequence may also include, or be an enhancer alone and/or other regulatory elements which are required for expression of the gene product.

In certain instances the promoter may comprise enhancer elements, exons, and introns from one or a variety of viruses and animals, and thereby the term “promoter” shall be understood to not be limited to being a non-expressed sequence, nor exclude a non-expressed sequence that is between expressed sequences (i.e. introns), nor be limited to exclude an enhancer alone so long as the combination of sequences used to construct the promoter are capable of initiating the specific transcription of a polynucleotide sequence.

A “constitutive” promoter is a nucleotide sequence which, when operably linked with a polynucleotide that encodes or specifies a gene product, causes the gene product to be produced in a cell under most or all physiological conditions of the cell and without requiring the addition of exogenous factors or the introduction of a different phenotype to the cell. This constitutive promoter can be cell-specific so long as it is produced in the specific, or target, cell under most or all physiological conditions of the cell. For example, a telencephalic neuronal-specific promoter is calcium/calmodulin-dependent protein kinase II (CaMKII). The CAG promoter and an elongation factor 1 (EF1) promoter are examples of constitutive promoters in a broad range of target cell types.

An “inducible” promoter is a nucleotide sequence which, when operably linked with a polynucleotide which encodes or specifies a gene product, causes the gene product to be produced in a cell substantially only when an inducer which corresponds to the promoter is present in the cell. For example, the promoter in a cyclooxygenase-2 gene is considered to be an inducible promoter in the periphery.

As used herein, the term “recombinant cell” refers to a cell into which an exogenous DNA segment, such as DNA segment that leads to the transcription of a biologically-active polypeptide or production of a biologically active nucleic acid such as an RNA, has been introduced.

A “target gene” refers to a nucleic acid encoding a target protein to be expressed within a target cell upon entry of the vector carrying the target gene into the cell. The target gene includes naturally occurring polymorphisms (i.e. variants) and man-made modifications to the wild-type gene so long as the target protein is still expressed. An example of such man-made modifications includes codon-optimization.

A “target protein” refers to a man-made or naturally occurring protein of interest to be introduced by vector into a host cell. One some embodiments, the target protein, as encoded in the genome of the host cell, is not functional because of a polymorphism in the gene sequence resulting in some mistranscription, missense, or mistranslation of the gene whereby reduced or no target protein or inoperable target protein is produced (i.e. a polymorphism results in an early stop codon) or an attenuation in the activity of the target protein, as encoded by and expressed from the genome of a subject.

In some embodiments, the target protein comprises galactose-1-phosphate uridylyltransferase (GALT). GALT is the enzyme that interconverts (i.e. a reversible enzymatic reaction) uridine diphosphate-glucose and galactose-1-phosphate into glucose-1-phosphate and uridine diphosphate-galactose (i.e. the second step of the Leloir pathway of galactose metabolism). It is to be understood and contemplated that “GALT” encompasses naturally-occurring versions (i.e. human GALT) and non-naturally occurring GALT (i.e. amino-acid additions, deletions, or substitution of GALT, which increase or decrease the activity compared to that of naturally occurring GALT) so long as the enzyme referred to as GALT has at least the above-noted enzymatic activity. In some embodiments, the non-naturally occurring GALT has at least, or no more than, 5%, 10%, 15%, 20%, 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, 195%, or 200% of the activity of the corresponding naturally-occurring GALT, wherein “corresponding” contemplates and provides support for that to which the additions, deletions, or substitutions were applied. In embodiments, the GALT is a human GALT and, in some embodiments, has the amino acid sequence of SEQ ID NO:1. In alternate embodiments, the GALT has an amino acid sequence that has at least 99%, 95%, 90%, 85% or 80% sequence identify to SEQ ID NO:1 and has GALT activity. In other embodiments, the hGALT is encoded by the nucleotide sequence of SEQ ID NO: 2, or a nucleotide sequence that is at least 99%, 95%, 90%, 85% or 80% identical to SEQ ID NO: 2 and encodes hGALT having the amino acid sequence of SEQ ID NO: 2 or an hGALT which has an amino acid sequence that has at least 99%, 95%, 90%, 85% or 80% sequence identify to SEQ ID NO: 1 and has GALT activity

In some embodiments, the subject is deficient in GALT, as encoded by and expressed from the genome of the subject, due for example to an autosomal recessive inheritance of two defective GALT genes. In some embodiments, the subject has classic galactosemia. In some embodiments, the subject has an attenuated activity in GALT. In some embodiments, the subject has Duarte galactosemia. In some embodiments, the subject has an unstable form of the GALT enzyme, whether it is a polymorphism causing lower activity in the enzyme in comparison to that of individuals who do not have the polymorphism. In some embodiments, the subject has a mutation in the promoter regulating GALT transcription, which causes reduced transcription of mRNA encoding GALT and thereby, potentially, reduced expression of GALT (i.e. reduced GALT protein). In some embodiments, the subject has the combination of one gene encoding a deficiency in GALT activity and another gene encoding an attenuation in GALT activity. That is in some embodiments, the subject's genotype is heterozygous for the classical variant and the Duarte variant. In some embodiments the subject has one or both of a mutation from A to G in exon 6 of the GALT gene, which changes Glu188 to an arginine, and a mutation from A to G in exon 10, which changes Asn314 to an aspartic acid.

A “tissue-specific” promoter is a nucleotide sequence which, when operably linked with a polynucleotide encodes or specified by a gene, causes the gene product to be produced in a cell substantially only if the cell is a cell of the tissue type corresponding to the promoter.

A “vector” is a nucleic acid capable of delivering a target gene to the interior of a cell, and includes not only the expression-region (i.e. a promoter and a nucleic acid encoding a protein or even a nucleic acid), but also some cis-acting genetic component. The cis-acting genetic component provides for packaging within a virion, expression in a cell, replication in a cell, or a combination thereof.

By way of example, inverted-terminal repeats (ITRs) from adeno-associated viruses (AAVs) constitute a vector when adjoined to the nucleic acid encoding a target protein because the ITRs will provide for the nucleic acid encoding the target protein to be packaged within an AAV virion. ITRs also provide other cis-acting functions for expression of the nucleic acid encoding the target protein in the host cell upon entry of the vector into the host cell. Such cis-acting functions of ITRs include aiding in concatemer formation for genomic insertion; initiation of second strand formation in the case of a single-stranded (ss) AAV (ssAAV) vector; or initiation of replication and transcription in the case of ssAAV and self-complementary (sc) AAV (scAAV) vectors. In this regard, the AAV ITRs can be characterized based on the nucleic acid sequences providing such cis-acting functions from the serotypes of AAVs. That is, an ITR isolated from an AAV2 serotype can be known as an AAV2 ITR, even though the ITR generally does not contribute to the serotype of an AAV.

Further it is understood that although the scAAV ITR (e.g. SEQ ID NO.: 13) was developed by mutating or altering a wild type AAV2 ITR (e.g. SEQ ID NO.: 11, which is the 5′ flanking ITR) and specifically the terminal resolution site (trs) in the D-sequence, which is responsible for signaling for packaging (a packaging sequence), the scAAV ITR provides for a function not provided for by the wild-type AAV ITR, which is the generation of an AAV vector comprising an expressing region and a reverse complement thereof prior to packaging within the AAV virion. Without wishing to be bound to a particular theory, it is believed that when the producing cells express the AAV vector plasmid, an AAV vector comprising the expressing region and the two ITRs, one of which is the scAAV ITR, is produced. Because of the negating mutation to the trs in the D-sequence, DNA polymerase is able to polymerize from the expressing region and the remaining ITR their reverse complements. The scAAV vector is thereby obtained. In some embodiments, the scAAV vector is then packaged into a recombinant scAAV virion.

The scAAV vector may exhibit at least, or no more than, 50%, 60%, 70%, 80%, 90%, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 110-fold, 120-fold, 130-fold, or 140-fold more efficient transduction than a corresponding vector comprising a wild-type AAV2 ITR and no complement of the expressing region. Without wishing to be bound to a particular theory, it is believed that the host-cell synthesis of a double-stranded segment in a ssAAV vector is rate limiting in ssAAV vector transduction, and that by providing an expressing region and the reverse complement thereof said scAAV ITR (and vector comprising said scAAV ITR) provides for the above-noted increase in efficiency of transduction.

By way of example, a plasmid can comprise an origin of replication (e.g., ori from cytomegalovirus) which allows for the replication of the target gene within a cell, and such a plasmid is thereby a vector. A viral genetic code may provide a nucleic acid sequence or protein encoded therein that allows for insertion of the gene of interest into the host genome, thereby providing for the replication of the target gene during the replication of, and within, the host cell's genome.

“Expression vector” refers to a vector comprising an expressing region. An expressing region includes a recombinant polynucleotide comprising a nucleic acid that controls expression (i.e. a promoter) and a nucleic acid that encodes. The nucleic acid that encodes includes a nucleic acid that encodes a protein. Generally, the promoter is operatively linked to the nucleic acid that encodes the target protein in a manner that is capable of promoting expression of the protein upon entry of the vector into the host cell. In some embodiments, the promoter can be operably linked by ensuring that there is not codon misalignment.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The nucleotide and amino acid sequences provided herein are set out in Table 1.

### AAV Vectors

In some embodiments, a recombinant adeno-associated virus (AAV) vector is provided, which comprises an expressing region and at least two inverted-terminal repeats (ITRs); the expressing region comprises a promoter and a nucleic acid that encodes a galactose-1-phosphate uridylyl transferase (GALT); the promoter is operably linked to the expression of GALT; and the at least two ITRs flank the expressing region, the expressing region may further include other regulatory sequences such as enhancers, polyA signals, intron sequences and WPRE sequences.

In some embodiments, the GALT comprises an amino acid sequence comprising at least, or no more than, 85%, 86%, 87%, 88%, 89%, 90%, 91%, 92%, 93%, 94%, 95%, 96%, 97%, 98%, 99.0%, 99.1%, 99.2%, 99.3%, 99.4%, 99.5%, 99.6%, 99.7%, 99.8%, or 99.9% identity to; or 1, 2. 3, 4, 5, 6, 7, 8, or 9 substitutions, additions, deletions, or combinations thereof within; the amino acid sequence of SEQ ID NO.: 1. In some embodiments, the GALT comprises the amino acid sequence of SEQ ID NO.: 1. In some embodiments, the nucleic acid that encodes GALT comprises a nucleic acid having at least, or no more than, 85%, 86%, 87%, 88%, 89%, 90%, 91%, 92%, 93%, 94%, 95%, 96%, 97%, 98%, 99.0%, 99.1%, 99.2%, 99.3%, 99.4%, 99.5%, 99.6%, 99.7%, 99.8%, or 99.9% identity to; or 1, 2, 3, 4, 5, 6, 7, 8, or 9 substitutions, additions, deletions, or combinations thereof within; SEQ ID NO.: 2 or a reverse complementary sequence thereto. In some embodiments, the nucleic acid that encodes GALT comprises or consists of SEQ ID NO.: 2 or a reverse complementary sequence thereto.

In some embodiments, the ITR, or at least two ITRs, comprises an AAV1 ITR, AAV2 ITR, AAV3 ITR, AAV4 ITR, AAV5 ITR, AAV6 ITR, AAV7 ITR, AAV8 ITR, or an AAV9 ITR, and in embodiments may be an scAAV ITR. In some embodiments, the ITR, or at least two ITRs, comprises a AAV2 ITR or a scAAV ITR. In some embodiments, the ITR, or at least two ITRs, comprises AAV2 ITR and a scAAV ITR. In some embodiments, the scAAV ITR is an AAV2 ITR lacking at least one functional terminal resolution site (trs) in the D-sequence. In some embodiments, the scAAV ITR lacks at least one functional trs. In some embodiments, the scAAV ITR has at least one substitution, addition, or deletion in the at least one trs, wherein the at least one substitution, addition, or deletion confers lack of function to the at least one trs. In some embodiments, the AAV ITR has at least one a D-sequence deleted. In some embodiments, the D-sequence deleted is at the 3′ end of the ITR (ssD[−]). In some embodiments, the D-sequence deleted is a the 5′ end of the ITR (ssD[+]). In some embodiments, the ssD[−] sequence has at least one substitution, deletion, or addition that prevents binding of the 52-kDa-FK506-binding protein (FKBP52). In some embodiments, the AAV ITR has a D-sequence replaced with a transcription factor binding site. In some embodiments, the transcription factor binding site comprises an S-sequence. In some embodiments, the S-sequence comprises a Foxd3 binding site or a NF-μE1 binding site. In some embodiments, the transcription factor binding site or the S-sequence comprises a GATA-1 and GATA-2 binding site. In some embodiments, the ITR, or at least two ITRs, comprise a nucleic acid having at least, or no more than, 85%, 86%, 87%, 88%, 89%, 90%, 91%, 92%, 93%, 94%, 95%, 96%, 97%, 98%, 99.0%, 99.1%, 99.2%, 99.3%, 99.4%, 99.5%, 99.6%, 99.7%, 99.8%, or 99.9% identity to; or 1, 2, 3, 4, 5, 6, 7, 8, or 9 substitutions, additions, deletions, or combinations thereof within; SEQ ID NO.: 11, SEQ ID NO.: 13, or a reverse complementary sequence thereto, including SEQ ID NO: 12. In some embodiments, the ITR, or at least two ITRs, comprise SEQ ID NO.: 11, SEQ ID NO.: 12, or a reverse complementary sequence thereto. In fact the flanking ITRs may be reverse complements of each other such that the ITR at the 5′ end of the expression cassette has a nucleotide sequence of SEQ ID NO: 11 and the ITR at the 3′ end of the expression cassette has a nucleotide sequence of SEQ ID NO: 12 (or the reverse complement of each). In the case of a self-complementary vectors, the ITR at the 5′ end of the expression cassette has a mutant sequence of SEQ ID NO: 13 and the ITR at the 3′ end of the expression cassette is an unmodified ITR, for example, and AAV2 ITR having a nucleotide sequence of SEQ ID NO: 11 at the 5′ end of the expression cassette.

In some embodiments, the sequence encoding GALT is operably linked to a promoter, including a constitutive promoter. In some embodiments the promoter is a CAG promoter. A CAG promoter is a composite, synthetic promoter which contains the CMV early enhancer element, the chicken β-actin promoter and the first exon and first intron of the chicken β-actin gene and the splice acceptor of the rabbit 3 globin gene. See, e.g., Miyazaki et al, Gene 79:269-277 (1989) and Niwa et al, Gene 108:193-199 (1991). In certain embodiments, the CAG “promoter” has a nucleotide sequence of SEQ ID NO:9, or may be an at least 200, 300, 400, 500 or 600 nucleotide fragment thereof with promoter activity that promotes expression of the target gene in the appropriate tissues (or a reverse complement thereof as appropriate). In other embodiments, the promoter is an EF1a or EF1α promoter, which may or may not include the EF-1α intron A sequence. In certain embodiments, the EF1a or EF1α promoter has a nucleotide sequence of SEQ ID NO: 10 (or a nucleotide sequence of the first 230 nucleotides of SEQ ID NO: 10, which does not include the EF1α intron A. Alternately, the EF1α promoter is an at least 100, 200, 300, 400, 500, 600, 700, 800, 900 or 1000 nucleotide fragment of SEQ ID NO: 10 and has promoter activity that promotes expression of the target gene in the appropriate tissues (or a reverse complement thereof as appropriate). In some embodiments, the promoter comprises a nucleic acid sequence having at least, or no more than, 85%, 86%, 87%, 88%, 89%, 90%, 91%, 92%, 93%, 94%, 95%, 96%, 97%, 98%, 99.0%, 99.1%, 99.2%, 99.3%, 99.4%, 99.5%, 99.6%, 99.7%, 99.8%, or 99.9% identity to; or 1, 2, 3, 4, 5, 6, 7, 8, or 9 substitutions, additions, deletions, or combinations thereof within; SEQ ID NO.: 9, SEQ ID NO.: 10, or a reverse complementary sequence thereto and promotes GALT gene expression in the appropriate tissues. In some other embodiments, the promoter comprises or consists of a Rous sarcoma virus (RSV) LTR promoter, a cytomegalovirus (CMV) promoter, a simian virus (SV40) promoter, a dihydrofolate reductase promoter, a β-actin promoter, a phosphoglycerol kinase (PGK) promoter, a P5 promoter, a Ubc promoter, a tetracycline response element promoter, a UAS promoter, an Ac5 promoter, a polyhedrin promoter, a calmodulin-dependent protein kinase II-α (CaMKIIα) promoter, a galactose promoter, the GALT promoter, a GDS promoter, an alcohol dehydrogenase promoter, an H1 promoter, a U6 promoter, or an Alpha-1-antitrypsin promoter. In some embodiments, the β-actin promoter is a chicken β-actin (“CBA”) promoter or a human β-actin promoter.

In some embodiments, the expressing region is in an anti-sense (e.g. reverse complementary) orientation or in sense orientation. In some embodiments, the vector comprises two or more expressing regions. In some embodiments, the two or more expressing regions comprises one in antisense orientation and another in sense orientation (i.e. as an scAAV would).

In some embodiments, the expressing region further comprises a nucleic acid that encodes a polyadenylation (poly(A)) signal 3′ of the target gene coding sequence such that the expressed mRNA has a polyA tail. In some embodiments, the nucleic acid that encodes the poly(A) signal comprises a bovine growth hormone (bGH) poly(A) tail signal or a simian virus 40 (SV40) poly(A) tail signal. In some embodiments, the polyA signal has a nucleotide sequence of SEQ ID NO 15 (bGH polyA signal) or SEQ ID NO: 16 (SV40 polyA signal) (or a reverse complement thereof) or the polyA signal has at least, or no more than, 85%, 86%, 87%, 88%, 89%, 90%, 91%, 92%, 93%, 94%, 95%, 96%, 97%, 98%, 99.0%, 99.1%, 99.2%, 99.3%, 99.4%, 99.5%, 99.6%, 99.7%, 99.8%, or 99.9% identity to; or 1, 2, 3, 4, 5, 6, 7, 8, or 9 substitutions, additions, deletions, or combinations thereof within; SEQ ID NO.: 15, SEQ ID NO.: 16, or a reverse complementary sequence thereto.

In some embodiments, the expression cassette further comprises regulatory elements that may enhance the expression of the target gene. In one embodiment, the expressing region or expression cassette further comprises a woodchuck hepatitis virus post-transcriptional regulatory element (WPRE). In some embodiments, the WPRE is downstream (3′ of) the GALT coding sequence and upstream (5′ of) the poly(A) tail signal. In some embodiments, the WPRE comprises a nucleic acid having at least, or no more than, 85%, 86%, 87%, 88%, 89%, 90%, 91%, 92%, 93%, 94%, 95%, 96%, 97%, 98%, 99.0%, 99.1%, 99.2%, 99.3%, 99.4%, 99.5%, 99.6%, 99.7%, 99.8%, or 99.9% identity to; or 1, 2, 3, 4, 5, 6, 7, 8, or 9 substitutions, additions, deletions, or combinations thereof within SEQ ID NO.: 14 (or reverse complement thereof) that enhances target gene expression. In some embodiments, the WPRE comprises SEQ ID NO.: 14 (or a reverse complement thereof). In other embodiments, the expression cassette comprises an intron or a chimeric intron sequence that promotes target gene expression. The intron sequence may be inserted between the promoter and the hGALT coding sequence. In certain embodiments, the intron has a nucleotide sequence of SEQ ID NO: 17 (or reverse complement thereof), or a nucleotide sequence having at least, or no more than, 85%, 86%, 87%, 88%, 89%, 90%, 91%, 92%, 93%, 94%, 95%, 96%, 97%, 98%, 99.0%, 99.1%, 99.2%, 99.3%, 99.4%, 99.5%, 99.6%, 99.7%, 99.8%, or 99.9% identity to; or 1, 2, 3, 4, 5, 6, 7, 8, or 9 substitutions, additions, deletions, or combinations thereof within SEQ ID NO.: 17 (or reverse complement thereof) that enhances target gene expression

In some embodiments, the recombinant AAV vector may be packaged into a recombinant AAV virion.

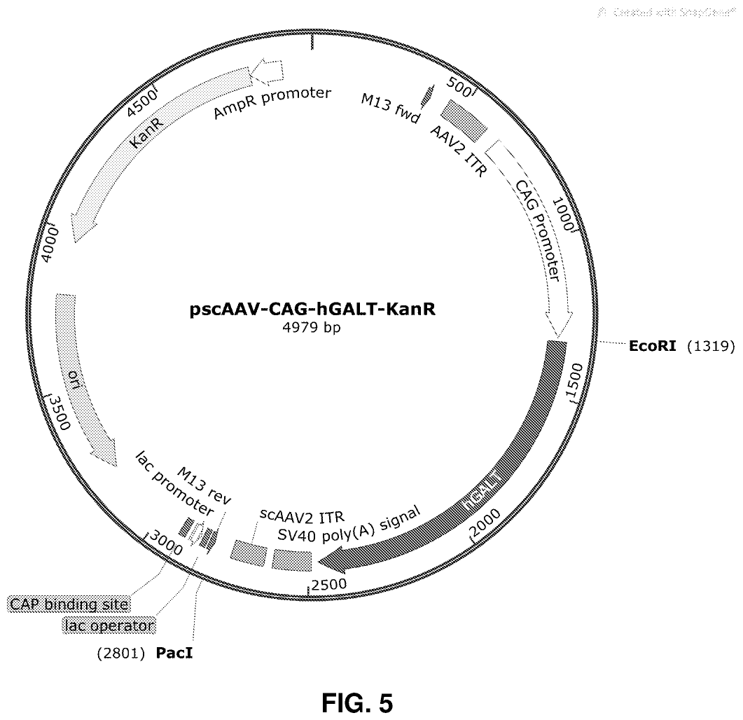

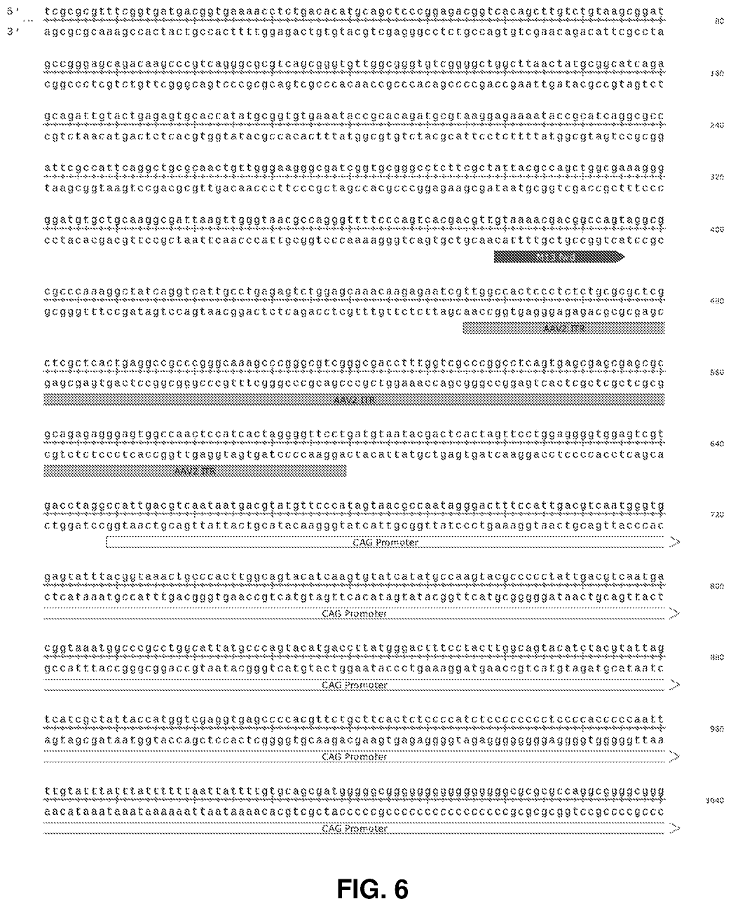

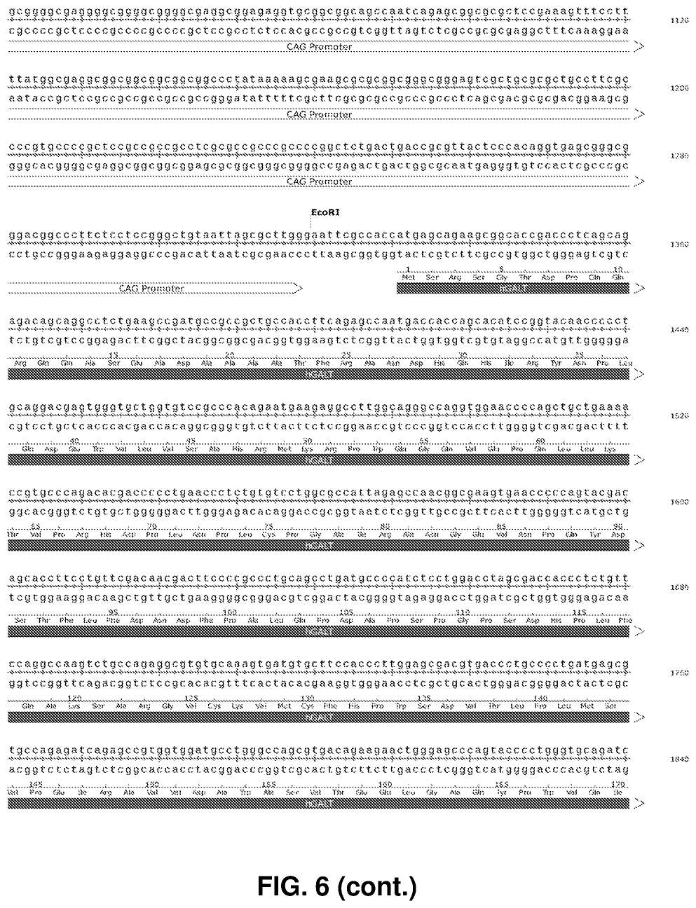

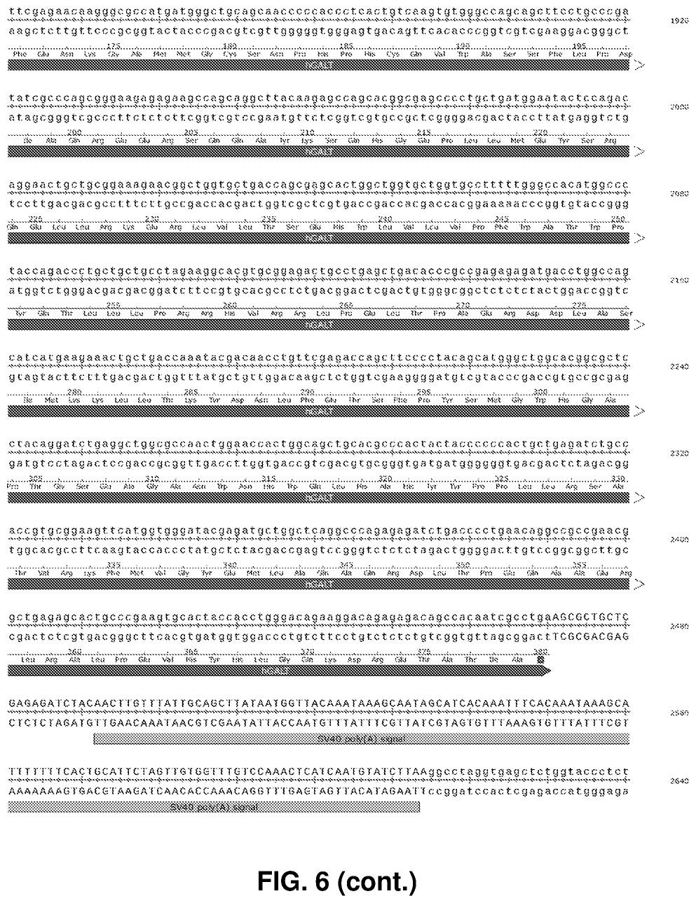

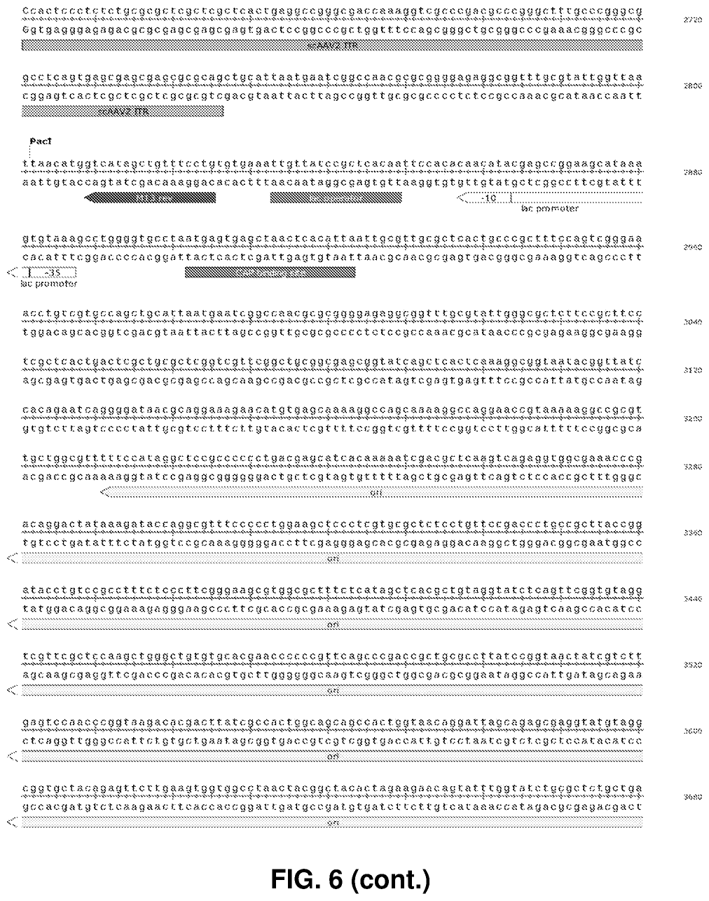

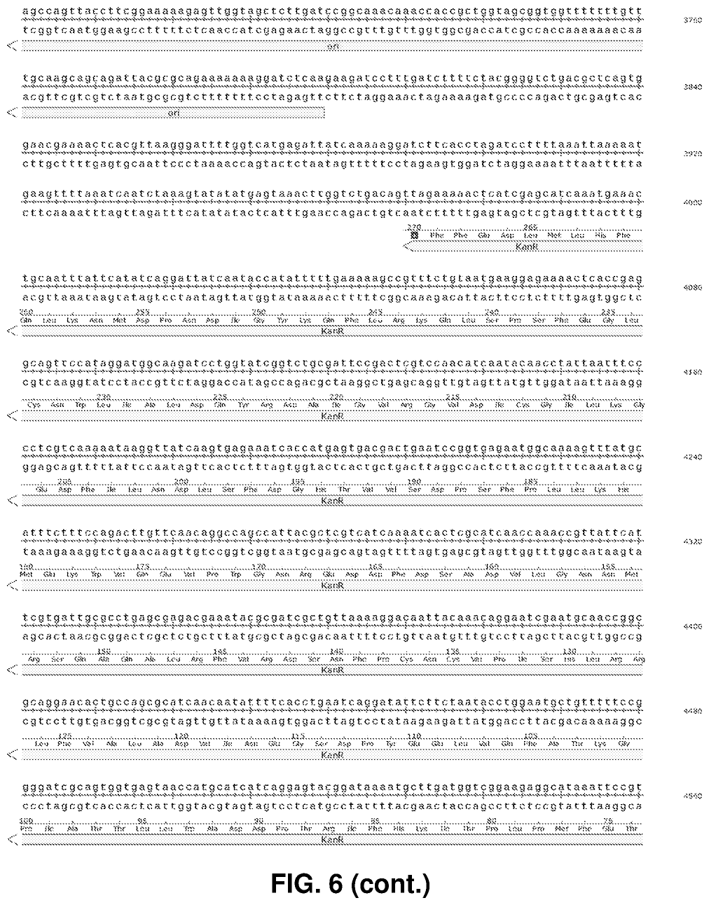

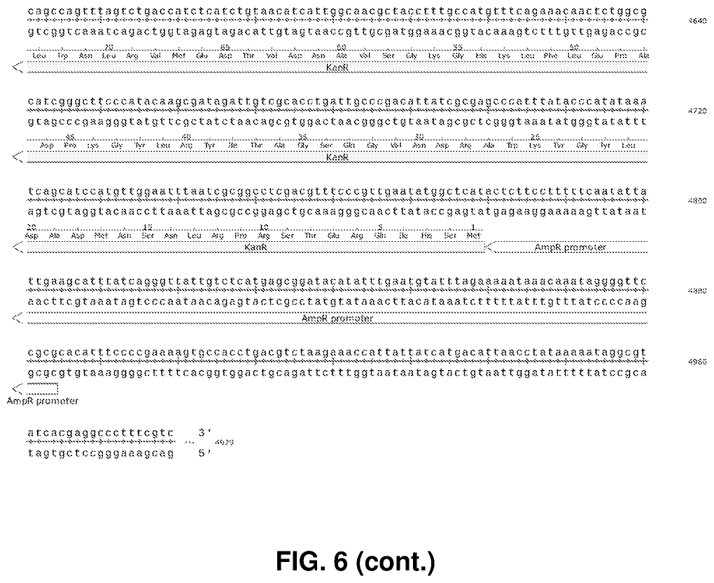

In some embodiments, a recombinant self-complementary AAV (scAAV) vector is provided. In some embodiments, the scAAV comprises the expressing region, at least three ITRs, and a nucleic acid reverse complementary to expressing region. In some embodiments, the at least three ITRs comprise at least one scAAV ITR. In some embodiments, the scAAV vector is in the following order: at least one of the three ITRs, the expressing region, the scAAV ITR, the nucleic acid reverse complementary to the expressing region, and at least one of the three ITRs. In some embodiments, the at least three ITRs comprise at least two scAAV ITRs. In some embodiments, the scAAV vector is in the following order: at least one of the at least two scAAV ITRs, the expressing region, at least one of the ITRs, the nucleic acid reverse complementary to the expressing region, and at least another of the at least two scAAV ITRs. In some embodiments, a scAAV vector plasmid is provided; the scAAV plasmid encoding the scAAV. In some embodiments, the scAAV vector plasmid encoding the scAAV will comprise at least two ITRs and the expressing region, wherein at least one of the at least two ITRs is the scAAV ITR. In embodiments, as discussed above, the 5′ ITR may have a nucleotide sequence of SEQ ID NO:11 and the modified ITR may be at the ′3 end of the expression cassette and have a nucleotide sequence of SEQ ID NO: 13. FIGS. 5 and 6 depict a plasmid and its nucleotide sequence, respectively, which can produce an scAAV.

Provided, thus, are expression cassettes that can be incorporated into an AAV vector for gene replacement expression of the target gene. In particular embodiments, the expression cassette may have elements arranged as follows: 5′AAV2ITR-CAG promoter sequence-hGALT coding sequence-SV40 polyA signal sequence-3′scAAV2ITR. In embodiments, the expression cassette has a nucleotide sequence of SEQ ID NO: 3. In other embodiments, the gene expression cassette has elements arranged as follows: 5′AAV2ITR-CAG Promoter (CMV enhancer-CBA promoter-Intron sequence)-hGALT coding sequence-WPRE sequence-bGH polyA signal sequence-3′AAV2ITR sequence. In embodiments, the expression cassette has a nucleotide sequence of SEQ ID NO: 4 (or the reverse complement thereof). In another embodiment, the expression cassette may have elements arranged as follows: 5′ITR-EF1α promoter sequence-hGALT coding sequence-WPRE sequence-bGH polyA signal sequence-3′AAV2 ITR. In embodiments, the expression cassette has a nucleotide sequence of SEQ ID NO: 5 (or reverse complement thereof). In embodiments, the expression cassette has a nucleotide sequence of SEQ ID NO:5 (or the reverse complement thereof).

In some embodiments, the scAAV vector is then packaged into a recombinant AAV virion.

In some embodiments, the expressing region comprises a nucleic acid having at least, or no more than, 85%, 86%, 87%, 88%, 89%, 90%, 91%, 92%, 93%, 94%, 95%, 96%, 97%, 98%, 99.0%, 99.1%, 99.2%, 99.3%, 99.4%, 99.5%, 99.6%, 99.7%, 99.8%, or 99.9% identity to; or 1, 2, 3, 4, 5, 6, 7, 8, or 9 substitutions, additions, deletions, or combinations thereof within; SEQ ID NO.: 3, SEQ ID NO.: 4, SEQ ID NO.: 5, SEQ ID NO.: 19, SEQ ID NO.: 20, or SEQ ID NO.: 21 or a reverse complementary sequence thereto, and is an expression cassette that expresses hGALT in appropriate human tissues. In some embodiments, the expressing region or expression cassette comprises SEQ ID NO.: 3, SEQ ID NO.: 4, SEQ ID NO.: 5, SEQ ID NO.: 19, SEQ ID NO.: 20, or SEQ ID NO.: 21 or a reverse complementary sequence thereto.

### AAV Vector Plasmids

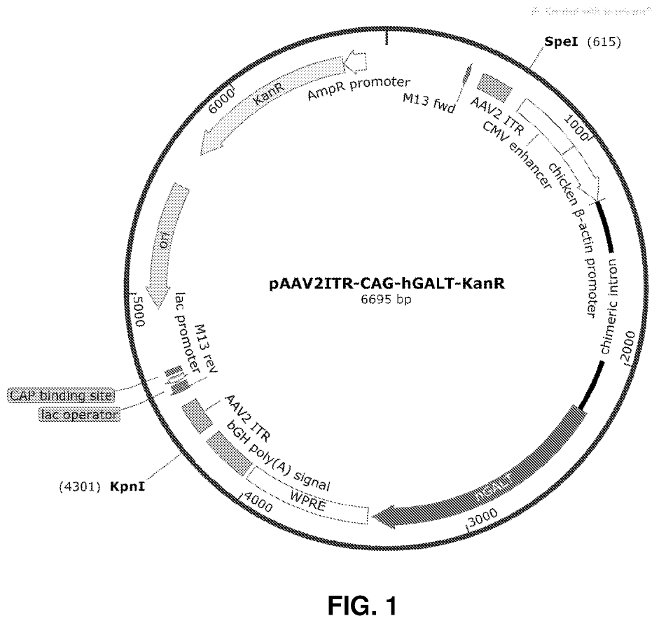

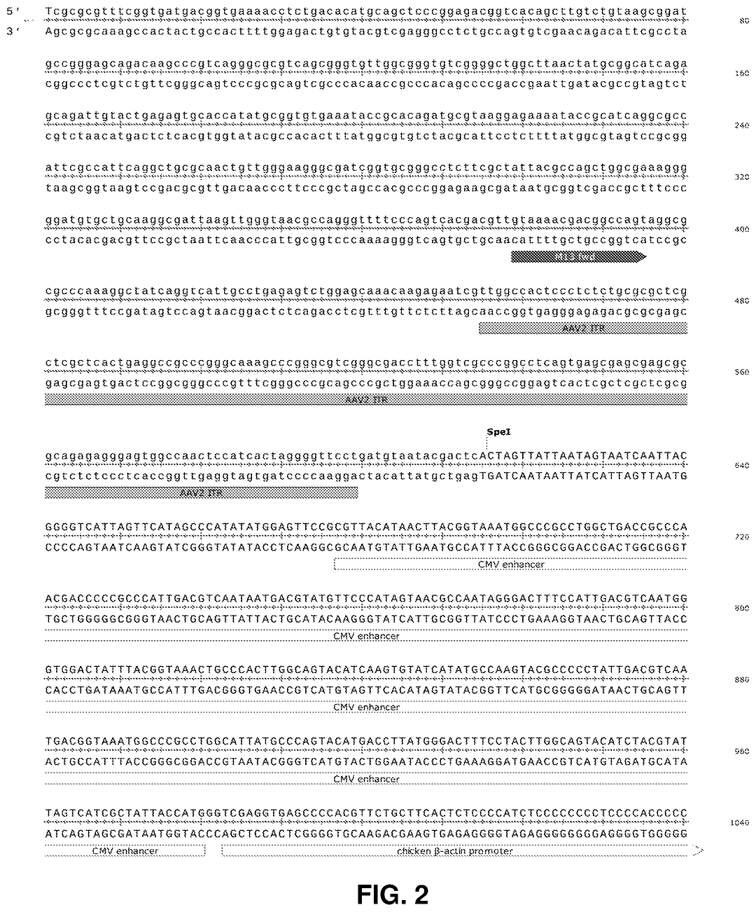

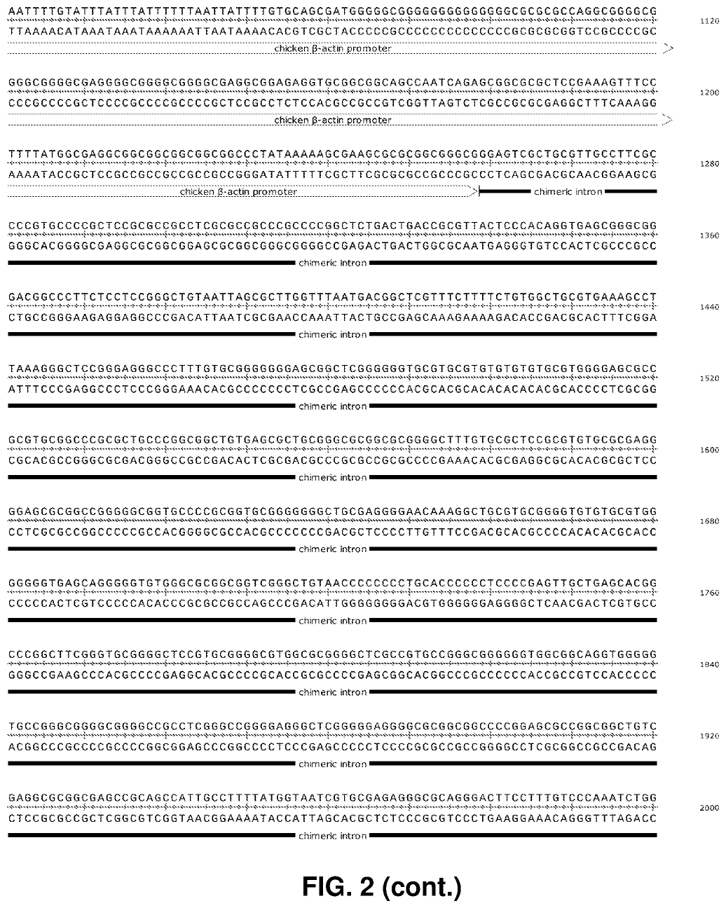

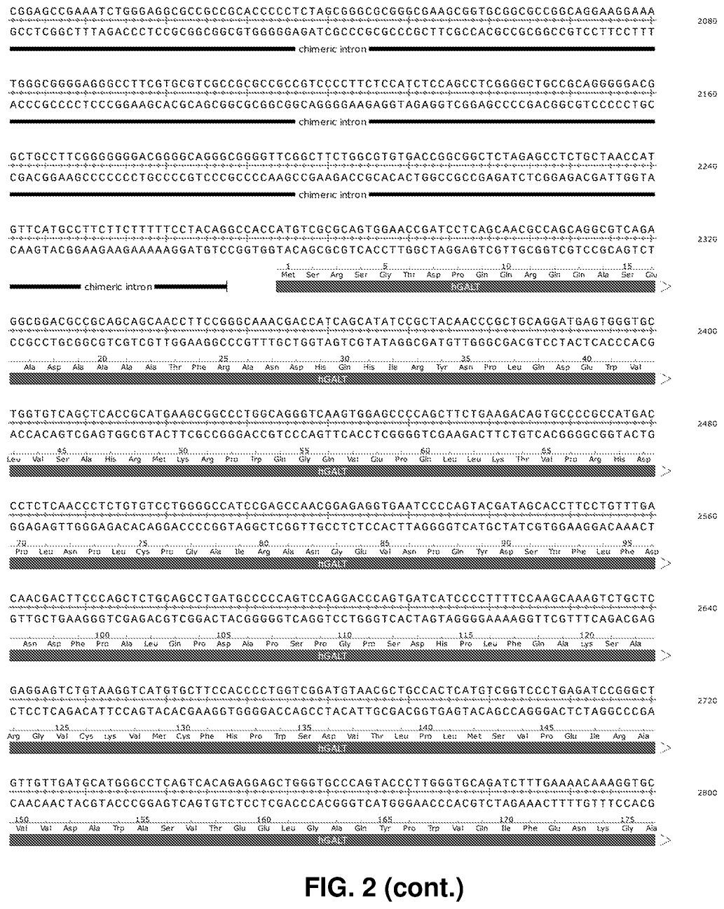

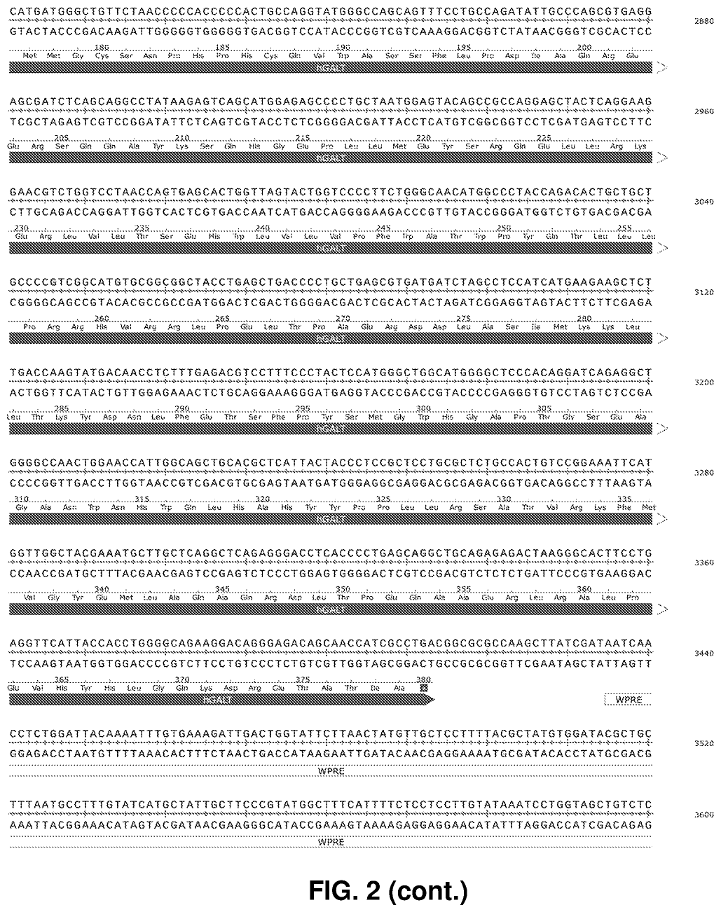

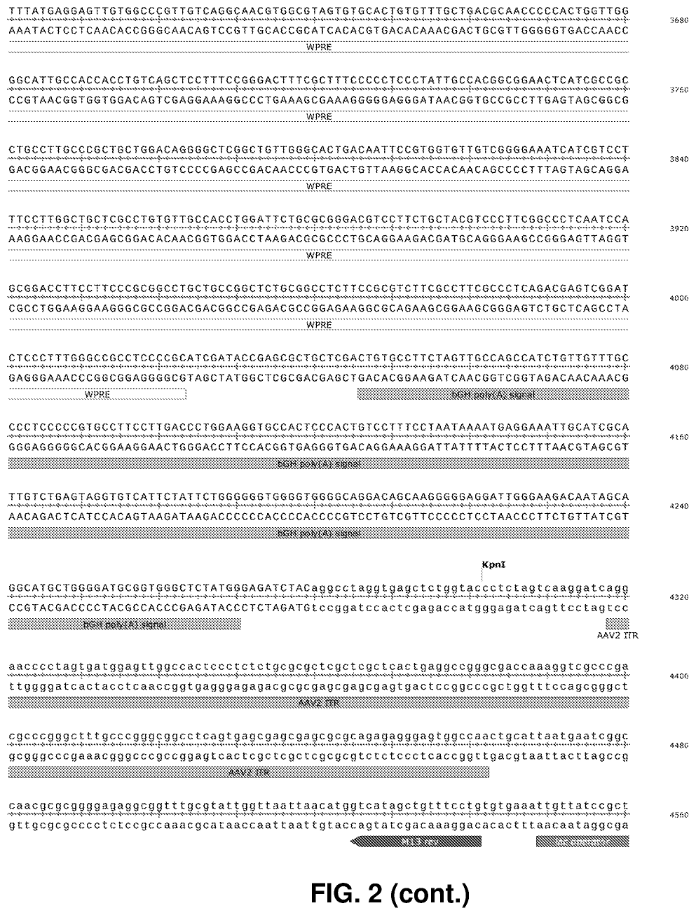

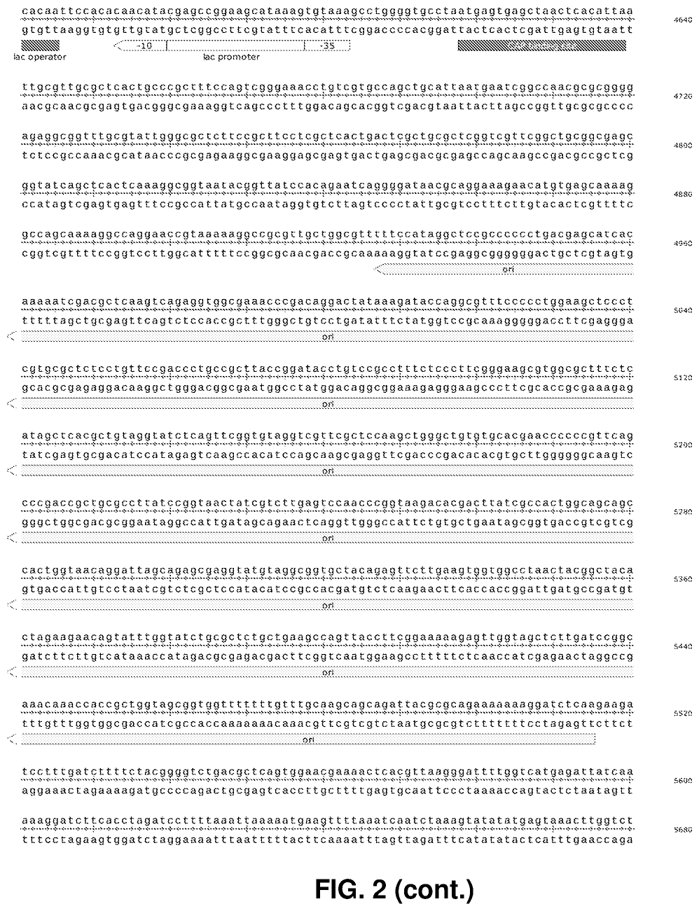

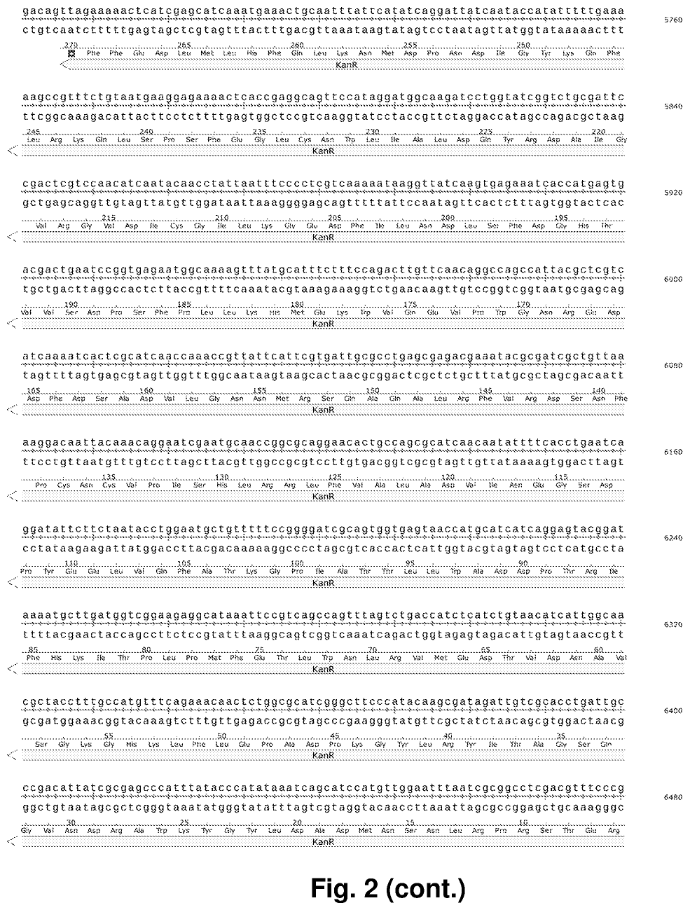

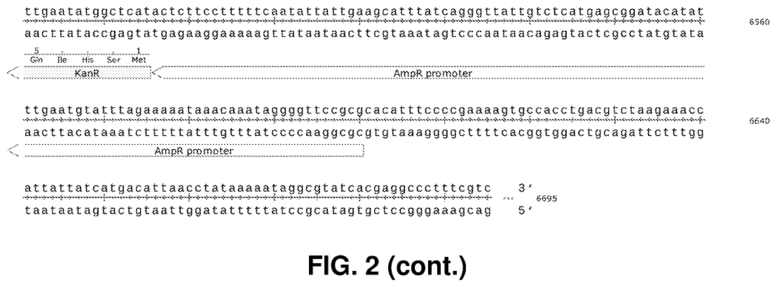

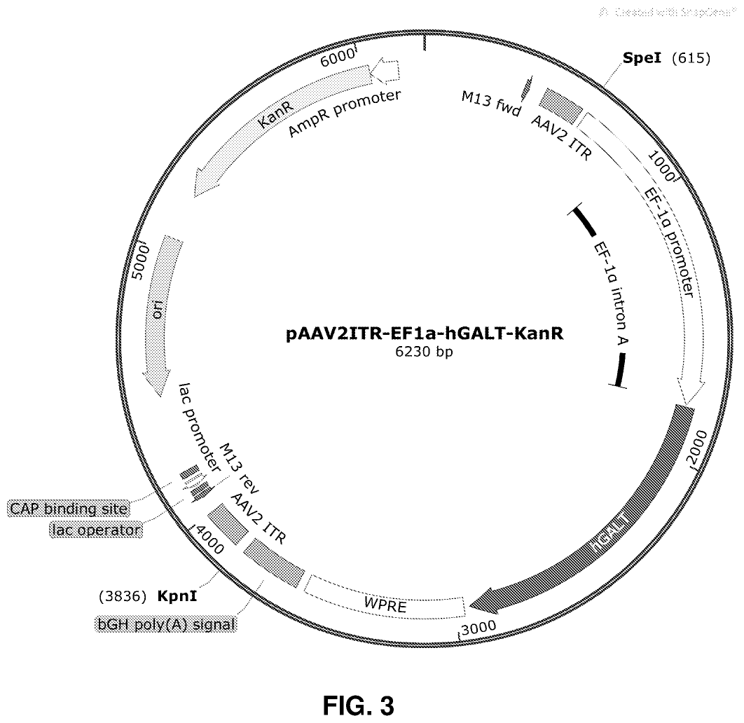

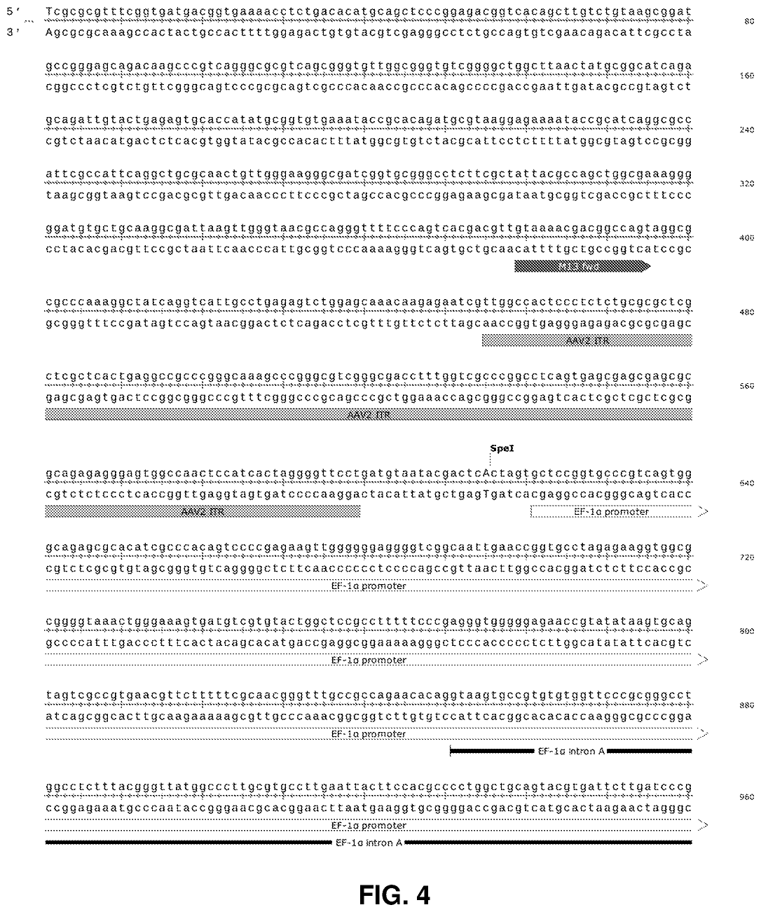

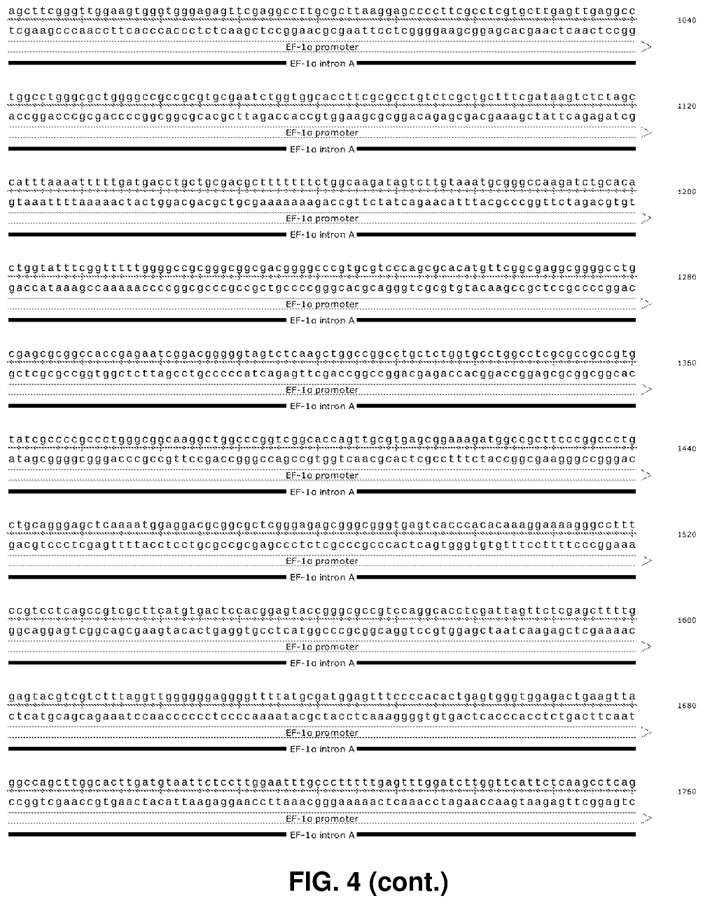

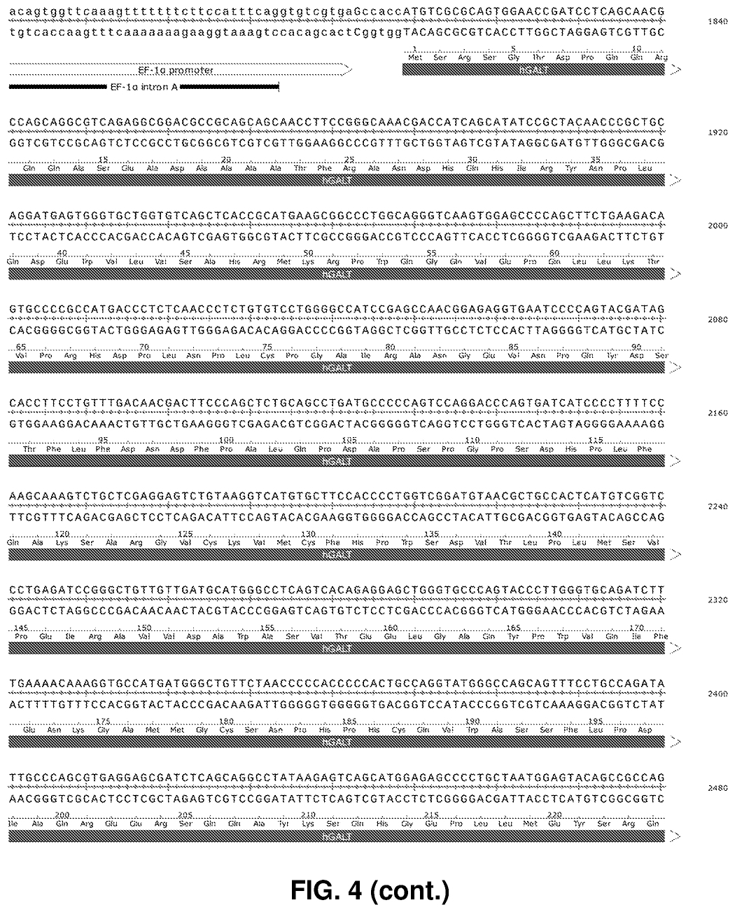

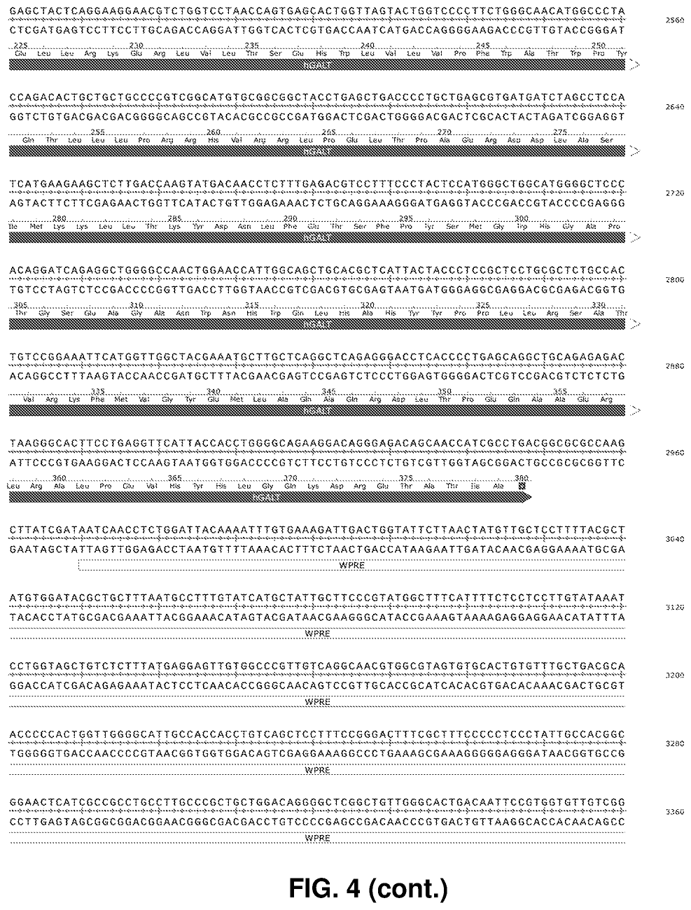

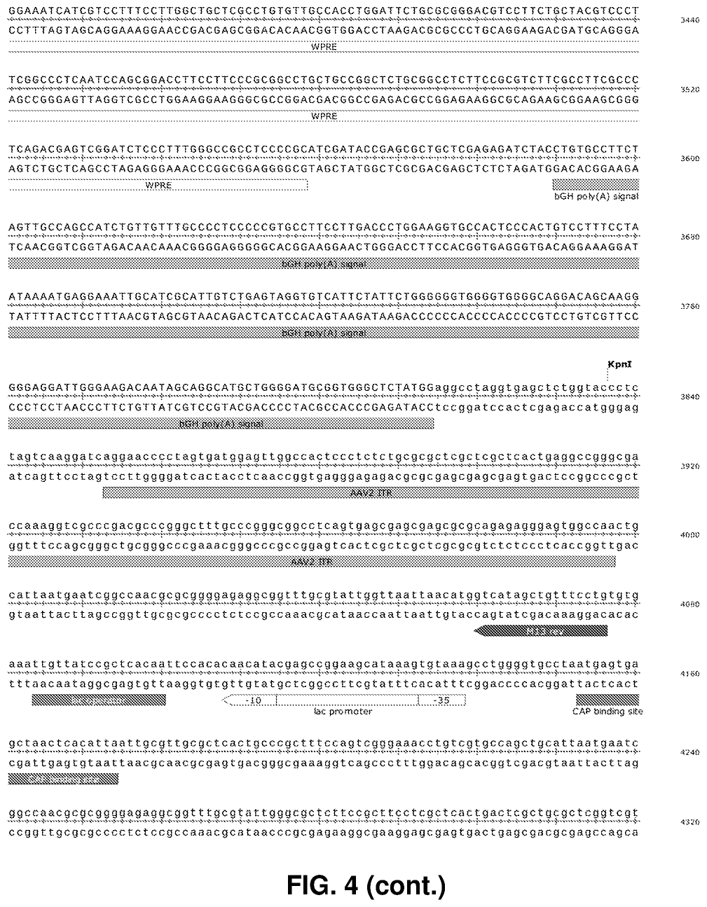

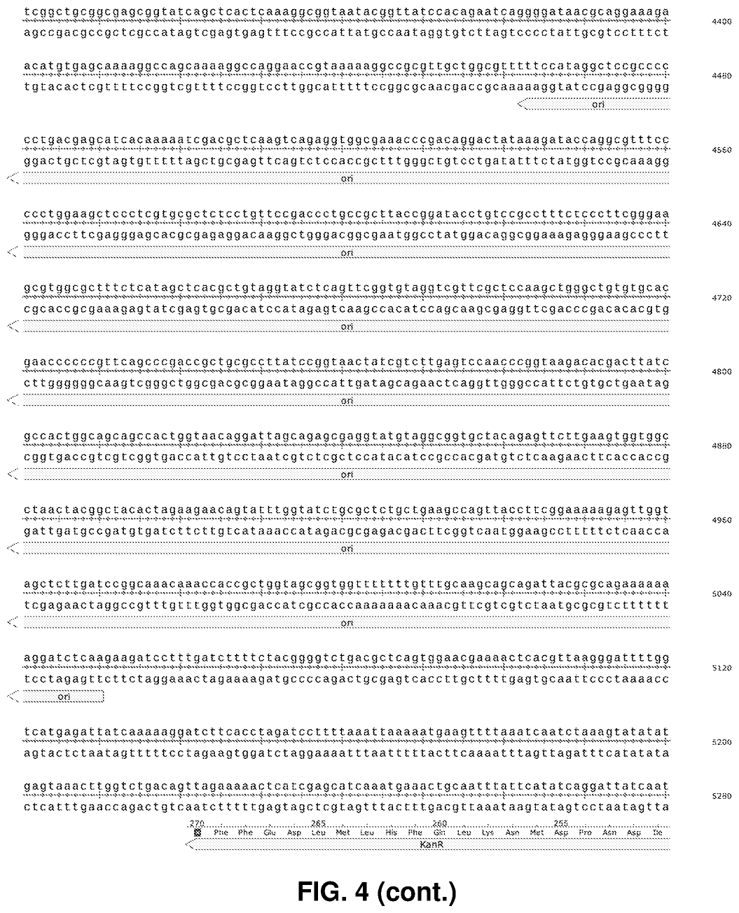

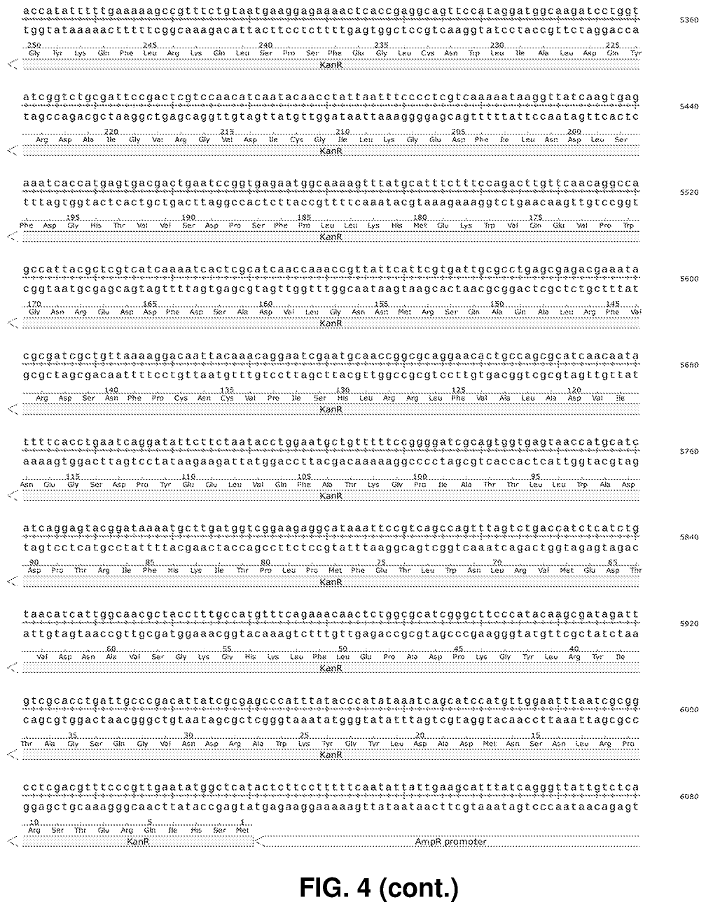

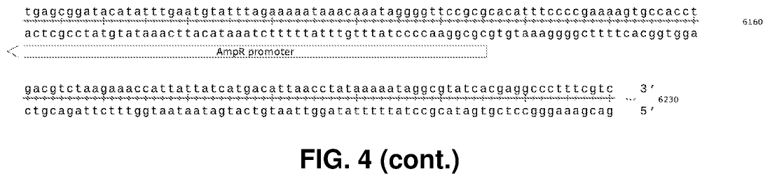

In some embodiments, an AAV vector plasmid is provided that may be used to prepare a recombinant AAV viral particle having a recombinant genome comprising a nucleotide sequence encoding the target gene operably linked to regulatory elements that promote expression in appropriate tissues. The plasmids provided herein generally have an origin of replication and selectable markers to permit reproduction of the plasmid and use in host cells for generating the recombinant AAV viral particles described herein. Exemplary plasmids, and their sequences, as depicted in FIGS. 1-6. The plasmids provided herein include plasmids comprising the expression cassettes described herein. In particular, the AAV vector plasmids may comprise an expression cassette having a nucleotide sequence of SEQ ID NO: 3, 4 or 5. In certain embodiments, the AAV vector plasmid comprises a nucleic acid having at least, or no more than, 85%, 86%, 87%, 88%, 89%, 90%, 91%, 92%, 93%, 94%, 95%, 96%, 97%, 98%, 99.0%, 99.1%, 99.2%, 99.3%, 99.4%, 99.5%, 99.6%, 99.7%, 99.8%, or 99.9% identity to; or 1, 2, 3, 4, 5, 6, 7, 8, or 9 substitutions, additions, deletions, or combinations thereof within; SEQ ID NO.: 3, SEQ ID NO.: 4, SEQ ID NO.: 5, SEQ ID NO.: 19, SEQ ID NO.: 20 or SEQ ID NO.: 21. An AAV vector plasmid may have a nucleotide sequence of SEQ ID NO.: 6, SEQ ID NO.: 7, or SEQ ID NO.: 8.

In some embodiments, the AAV vector plasmid further comprises a bacterial expressing region. In some embodiments, the bacterial expressing region comprises a bacterial promoter and a nucleic acid that encodes a bacterial selecting region. In some embodiments, the nucleic acid that encodes the bacterial selecting region is operably linked to the bacterial promoter. In some embodiments, the nucleic acid that encodes the bacterial selecting region comprises a nucleic acid that encodes an antibiotic resistance gene or protein. In some embodiments, the antibiotic resistance gene or protein comprises an ampicillin resistance gene (AmpR) or a kanamycin resistance gene sequence (KanR). In some embodiments the bacteria promoter comprises AmpR promoter or a KanR promoter. In some embodiments, the AAV vector plasmid further comprises an origin of replication. In some embodiments, the origin of replication comprises a CMV origin of replication (ori). In some embodiments, the AAV vector plasmid further comprises a eukaryotic expressing region. In some embodiments, the eukaryotic expressing region comprises a eukaryotic promoter and a nucleic acid that encodes a eukaryotic selecting region. In some embodiments, the nucleic acid that encodes a eukaryotic selecting region is operably linked to the eukaryotic promoter. In some embodiments, the eukaryotic promoter comprises nucleic catabolite activator protein (CAP) binding site or a lactose (lac) promoter. In some embodiments, the eukaryotic selecting region comprises a lac operator. In some embodiments the plasmid comprises an M13 reverse primer region.

### AAV Virion

In some embodiments, a recombinant AAV virion is provided. In some embodiments, the AAV virion comprises an AAV capsid protein and the recombinant AAV vector that comprises the nucleotide sequence encoding hGALT operably linked to regulatory elements. In some embodiments, the recombinant AAV vector is a recombinant scAAV vector. In some embodiments, the recombinant AAV or scAAV vector is a single-stranded DNA. In some embodiments, the AAV capsid protein encapsulates the recombinant AAV vector.

In some embodiments, the AAV capsid protein comprises a VP1, a VP2, and a VP3. The capsid preferably has tropism for appropriate cells and tissues, such as, for example, nervous tissue, CNS, liver, etc. In some embodiments, the capsid protein comprises an AAV1 capsid protein, an AAV2 capsid protein, an AAV3 capsid protein, an AAV4 capsid protein, an AAV5 capsid protein, an AAV6 capsid protein, an AAV7 capsid protein, an AAV8 capsid protein, an AAV9 capsid protein, an AAV-DJ capsid protein, an AAV-DJ/8 capsid protein, an AAV-Rh10 capsid protein, an AAV-retro capsid protein, an AAV-PHP.B capsid protein, an AAV8-PHP.eB capsid protein, or an AAV-PHP.S capsid protein. In certain embodiments, the rAAV particle has an AAV9 capsid protein, for example, having an amino acid sequence of SEQ ID NO:18. Alternatively, the capsid protein has an amino acid sequence that is 99%, 98%, 95%, 90% or 85% identical to the AAV9 capsid and has the tropism and transduction activity of the AAV9 capsid protein.

In some aspects, the isolated nucleic acids and/or rAAVs described herein can be modified and/or selected to enhance the targeting of the isolated rAAVs to a target tissue (e.g., CNS). Non-limiting methods of modifications and/or selections include AAV capsid serotypes (e.g., AAV9), tissue-specific promoters, and/or targeting peptides. In some aspects, the isolated nucleic acids and rAAVs disclosed herein can comprise AAV capsid serotypes with enhanced targeting to CNS tissues (e.g., AAV9). In some aspects, the isolated nucleic acids and rAAVs described herein can comprise tissue-specific promoters. In some aspects, the isolated nucleic acids and rAAVs described herein can comprise AAV capsid serotypes with enhanced targeting to CNS tissues and tissue-specific promoters. While AAV9 targets CNS tissue, the rAAV9 vectors may also transduce other non-CNS tissues and, thus, the transgenes, under the control of a promoter such as the CAG promoter may be expressed both in the CNS and other tissues outside the CNS.

Methods for obtaining recombinant AAVs having a desired capsid protein can be obtained from U.S. Patent Application Publication Number 2003/0138772, for example. Typically the methods involve culturing a host cell which contains a nucleic acid sequence encoding an AAV capsid protein or fragment thereof; a functional rep gene; sufficient helper functions to permit packaging of the recombinant AAV vector into the AAV capsid proteins; and a recombinant AAV vector plasmid comprising the AAV vector. Typically, capsid proteins are structural proteins encoded by the cap gene of an AAV. In some aspects, wherein the capsid protein comprises VP1, VP2, and VP3, said VP1, VP2, and VP3 are transcribed from a single cap gene via alternative splicing. In some aspects, the molecular weights of VP1, VP2 and VP3 are respectively about 87 kDa, about 72 kDa and about 62 kDa. In some aspects, upon translation, capsid proteins form a spherical 60-mer protein shell around the viral genome. In some aspects, capsid proteins protect a viral genome, deliver a genome and/or interact with a host cell. In some aspects, capsid proteins deliver the viral genome to a host in a tissue specific manner.

In some aspects, components to be cultured in the host cell to package a recombinant AAV vector in an AAV capsid can be provided to the host cell in trans. Alternatively, any one or more of the required components (e.g., recombinant AAV vector, rep sequences, cap sequences, and/or helper functions) can be provided by a stable host cell which has been engineered to contain one or more of the required components. In particular embodiments, provided are host cells comprising the recombinant AAV constructs or plasmids comprising the target gene sequence, a plasmid providing the AAV rep and cap gene sequences, and a construct providing adenoviral helper proteins as needed to produce the recombinant viral particle.

In some aspects, such a stable host cell can contain the required component(s) under the control of an inducible promoter. However, the required component(s) can be under the control of a constitutive promoter. Examples of suitable inducible and constitutive promoters are provided herein, in the discussion of regulatory elements suitable for use with the transgene. In some aspects, a selected stable host cell can contain selected component(s) under the control of a constitutive promoter and other selected component(s) under the control of one or more inducible promoters. For example, a stable host cell may be generated which is derived from 293 cells (which contain El helper functions under the control of a constitutive promoter), but which contain the rep and/or cap proteins under the control of inducible promoters. Still other stable host cells may be generated by one of skill in the art.

The recombinant AAV vector, rep sequences, cap sequences, and helper functions useful for producing the rAAV described herein can be delivered to the packaging host cell using any appropriate genetic element (vector, e.g. plasmid). The selected genetic element can be delivered by any suitable method, including those described herein. The methods used to construct any of compositions disclosed herein are known to those with skill in nucleic acid manipulation and include genetic engineering, recombinant engineering, and synthetic techniques. See, e.g., . Sambrook et al, Molecular Cloning: A Laboratory Manual, Cold Spring Harbor Press, Cold Spring Harbor, N.Y. Similarly, methods of generating rAAV virions are well known and the selection of a suitable method is not a limitation on the present disclosure. Sec, e.g., K. Fisher et al, J. Virol., 70:520-532 (1993) and U.S. Pat. No. 5,478,745.

In some aspects, recombinant AAVs can be produced using the triple transfection method (described in detail in U.S. Pat. No. 6,001,650). Typically, the recombinant AAVs can be produced by transfecting a host cell with a recombinant AAV vector (comprising a transgene) to be packaged into AAV particles, an AAV helper function vector, and an accessory function vector. An AAV helper function vector encodes the “AAV helper function” sequences (i.e., rep and cap), which function in trans for productive AAV replication and encapsidation with the cap gene encoding the capsid proteins of desired serotype, for example, encoding the AAV9 capsid. In some aspects, the AAV helper function vector can support efficient AAV vector production without generating any detectable wild-type AAV virions (i.e., AAV virions containing functional rep and cap genes). Non-limiting examples of vectors suitable for use with the present disclosure include pHLP19, described in U.S. Pat. No. 6,001,650 and pRep6cap6 vector, described in U.S. Pat. No. 6,156,303, the entirety of both incorporated by reference herein. The accessory function vector encodes nucleotide sequences for non-AAV derived viral and/or cellular functions upon which AAV is dependent for replication (i.e., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpesvirus (other than herpes simplex virus type-1), and vaccinia virus.

Cells. Disclosed herein are transfected host cells. The term “transfection” is used to refer to the uptake of foreign DNA by a cell, and a cell has been “transfected” when exogenous DNA has been introduced through the cell membrane. Examples of methods of transfection include Graham et al. (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13:197. Such techniques can be used to introduce one or more exogenous nucleic acids, such as a nucleotide integration vector and other nucleic acid molecules, into suitable host cells.

In one aspect, a cell is provided. In some embodiments, the cell comprises an AAV second plasmid and an AAV vector plasmid. In some embodiments, the AAV second plasmid comprises rep and cap. In some embodiments, the cap encodes a VP1, a VP2, and a VP3. In some embodiments, the rep encodes rep78, rep68, rep 52, and rep 40. In some embodiments, the AAV vector plasmid comprises the recombinant AAV vector or the recombinant scAAV vector. In some embodiments, the cap is AAV9 cap. In some embodiments, the AAV vector plasmid comprises the expression cassette of SEQ ID NO.: 6, SEQ ID NO.: 7, or SEQ ID NO.: 8.

In another aspect, a method of producing the AAV virion is provided. In some embodiments, the method comprises transfecting a cell with a second plasmid and at least one of a vector plasmid or the AAV vector construct. In some embodiments, the vector plasmid or AAV vector construct comprises the recombinant AAV vector or the recombinant scAAV vector. In some embodiments, the second plasmid comprises cap and rep. In some embodiments, the cap encodes the VP1, the VP2, and the VP3. In some embodiments, the rep encodes rep78, rep68, rep 52, and rep 40. In some embodiments, the vector plasmid or the AAV vector construct comprises the expression cassette having the nucleotide sequence of SEQ ID NO.: 3, SEQ ID NO.: 4, SEQ ID NO.: 5, SEQ ID NO.: 19, SEQ ID NO.: 20 or SEQ ID NO.: 21 or having the nucleotide sequence of SEQ ID NO.: 6, SEQ ID NO.: 7, or SEQ ID NO.: 8.

### Methods of Use

In some embodiments, the AAV virion will transduce the cells, or the cells of the tissue or organ. In some embodiments, upon transduction, the AAV vector will be released from encapsulation from the capsid protein. In some embodiments, the AAV vector will be released into the cytosol or nucleus of the cell. In some embodiments, the cell's transcription machinery will bind to the promoter of the AAV vector. In some embodiments, the cell will express the nucleic acid encoding the protein. In some embodiments, the cell will express the protein. In some embodiments, the cell will express the nucleic acid encoding the GALT. In some embodiments, the cell will express the GALT. In some embodiments, the GALT will undergo its enzymatic activity (i.e. conduct at least the forward reaction or the reverse reaction of the interconversion of uridine diphosphate-glucose and galactose-1-phosphate into glucose-1-phosphate and uridine diphosphate-galactose).

In some embodiments, a method for treating at least one of galactosemia, insufficient galactose metabolism, GALT-deficiency, GALT-insufficiency, or symptoms of insufficient galactose metabolism in a subject in need thereof are provided. In some embodiments, a method of increasing galactose metabolism in a subject is provided. In some embodiments, a method of reducing a disease condition in a subject, who suffers from galactosemia, are provided. In some embodiments, said disease condition comprises jaundice, hepatosplenomegaly, hepatocellular insufficiency, hypoglycemia, renal tubular dysfunction, muscle hypotonia, sepsis, cataract, ataxia, tremor, decreased bone density, or primary ovarian insufficiency. In some embodiments, the method comprises administering to the subject a therapeutically effective amount of the AAV virion.

In some embodiments, the subject has a GALT, which comprises a substitution of at least one of: Q188R. N314D, L218L, S135L, K285N, L195P, T138M, Y209C, and IVS2-2A>Ga. In some embodiments, the subject has a deletion of about 5 kg in one or both of the genes encoding GALT, wherein the deletion first identified in individuals of Ashkenazi Jewish ancestry. In some embodiments, the subject has classic galactosemia or Duarte galactosemia.

In some embodiments, the administering or treating comprises: intravenous administration, intra-arterial, intramuscular administration, intracardiac administration, intrathecal administration, subventricular administration, epidural administration, intracerebral administration, intracerebroventricular administration, sub-retinal administration, intravitreal administration, intraarticular administration, intraocular administration, intraperitoneal administration, intrauterine administration, intradermal administration, subcutaneous administration, transdermal administration, transmucosal administration, or administration by inhalation. In some embodiments, the method comprises contacting the AAV virion, or an effective amount thereof, with at least one of the liver, the spleen, a muscle, a kidney, the blood, a lens, an eye, the cerebellum, the brainstem, the basal ganglia, the hypothalamus, the preoptic area, a hippocampus, a striatum, a cortex, a motor cortex, a prefrontal cortex, a somatosensory cortex, a temporal cortex, a visual cortex, an occipital lobe, a temporal lobe, a parietal lobe, a frontal lobe, a bone, a reproductive organ, an ovary, a testis, a skin surface, a prostate, a uterus, or a pancreas of the subject.

In some embodiments, the administration, treating, contacting, or effective amount comprises at least, or no more than, 10⁴, 10⁵, 10⁶, 10⁷, 10⁸, 10⁹, 10¹⁰, 10¹¹, 10¹², 10¹³, 10¹⁴, or 10¹⁵ plague forming units (PFU), PFU/mL, or virions of the AAV.

In some embodiments, the administering or treating can comprise at least, or no more than, 1, 2, 3, 4, 5, 6, 7, 8, 9, or 10 administrations or treatments. In some embodiments, successful treatment and/or repair is determined when one or more of the following is detected: alleviation or amelioration of one or more of symptoms of the treated subject's disease, disorder, or condition, diminishment of extent of the subject's disease, disorder, or condition, stabilized (i.e., not worsening) state of a disease, disorder, or condition, delay or slowing of the progression of the disease, disorder, or condition, and amelioration or palliation of the disease, disorder, or condition. In some embodiments, success of treatment is determined by detecting the presence repaired target polynucleotide in one or more cells, tissues, or organs isolated from the subject. In some embodiments, success of treatment is determined by detecting the presence polypeptide encoded by the repaired target polynucleotide in one or more cells, tissues, or organs isolated from the subject.

In some embodiments, the recombinant AAV (rAAV) virion can be administered in a composition, which optionally comprises a suitable carrier. Suitable carriers can be selected for the indication for which the rAAV is directed. For example, one suitable carrier includes saline, which may be formulated with a variety of buffering solutions (e.g., phosphate buffered saline). Examples of other suitable carriers include but are not limited to sterile saline, lactose, sucrose, calcium phosphate, gelatin, dextran, agar, pectin, peanut oil, sesame oil, and water. Optionally, the compositions disclosed herein can also include, in addition to the rAAV virion and carrier(s), other pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

In particular embodiments, the rAAV virion is administered in a pharmaceutical composition comprising phosphate buffered saline (PBS), pH 7.3 and 0.001% of a pharmaceutically acceptable non-ionic surfactant, such as, for example, pluronic F-68 (PF68), or other appropriate pharmaceutically acceptable buffers or excipients. The formulation may be frozen until ready for use and then thawed and administered. In some embodiments, the pharmaceutical composition can comprise 10 mM Tris, 150 mM NaCl. 0.02% poloxamer 188, 1 mM MgCl₂, adjusted to a pH of 8.0. In some embodiments, the composition can further comprise sugar, sorbitol, or trehalose. In some embodiments, the sugar is at least, or no more than, 0.0%, 0.1%. 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, or 3.0% weight by weight, weight by volume, or mole by mole. In some embodiments, the sugars is sucrose, glucose, or lactose. In some embodiments, the sorbitol is at least, or no more than, 0.0%, 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, or 3.0% weight by weight, weight by volume, or mole by mole. In some embodiments, the trehalose is at least, or no more than, 0.0%. 0.1%, 0.2%, 0.3%, 0.4%, 0.5%, 0.6%, 0.7%, 0.8%, 0.9%, 1.0%, 1.1%. 1.2%, 1.3%, 1.4%, 1.5%, 1.6%, 1.7%, 1.8%, 1.9%, 2.0%, 2.1%, 2.2%, 2.3%. 2.4%. 2.5%. 2.6%, 2.7%, 2.8%, 2.9%, or 3.0% weight by weight, weight by volume, or mole by mole.

In some aspects, the compositions disclosed herein can comprise an rAAV virion alone, or in combination with one or more other viruses (e.g., a second rAAV virion encoding having one or more different transgenes). In some aspects, a composition can comprise 1, 2, 3, 4, 5, 6, 7, 8, 9, 10, or more different rAAVs virion each having one or more different transgenes.

Recombinant AAV virions can be administered in sufficient amounts to transfect the cells of a desired tissue and to provide sufficient levels of gene transfer and expression without undue adverse effects. In some aspects, acceptable routes of administration include, but are not limited to, direct delivery to the selected organ (e.g., injection into the liver, skeletal muscle), oral, inhalation (including intranasal and intratracheal delivery), intraocular, intravenous, intramuscular, subcutaneous, intradermal, intratumoral, and other parental routes of administration. In some aspects, the route of administration can be by intracerebroventricular injection. Routes of administration may be combined, if desired.

The dose of rAAV virions required to achieve a particular “therapeutic effect.” e.g., the units of dose in genome copies/per kilogram of body weight (GC/kg), the units of dose in genome copies per brain volume, and units of dose in genome copies per CSF volume, will vary based on several factors including, but not limited to: the route of rAAV virion administration, the level of gene or RNA expression required to achieve a therapeutic effect, the specific disease or disorder being treated, and the stability of the gene or RNA product.

In some embodiments, an effective amount of an rAAV virion can be an amount sufficient to target infect an animal, target a desired tissue. The effective amount will depend primarily on factors such as the species, age, weight, health of the subject, and the tissue to be targeted, and may thus vary among animal and tissue. For example, an effective amount of the rAAV virion can be in the range from about 1 ml to about 100 ml of solution containing from about 10⁶ to 10¹⁶ genome copies (e.g., from 1×10⁶ to 1×10¹⁶, inclusive). In methods disclosed herein, the therapeutically effective dose is between 6×10¹³ gc/kg to 6×10¹⁴ gc/kg, including 7×10¹³ gc/kg, 8×10¹³ gc/kg, 9×10¹³ gc/kg, 1×10¹⁴ gc/kg, 2×10¹⁴ gc/kg. 3×10¹⁴ gc/kg, 4×10¹⁴ gc/kg, or 5×10¹⁴ gc/kg (or alternatively, genome copies per brain volume, CSF volume or other measurement appropriate for ICV or ICM delivery). In some aspects, a dosage between about 10¹¹ to 10¹² per kg or appropriate measurement rAAV genome copies can be appropriate. In some aspects, a dosage of between about 10¹¹ to 10¹³ per kg or appropriate measurement rAAV genome copies can be appropriate. In some aspects, a dosage of between about 10¹¹ to 10¹⁴ per kg or appropriate measurement rAAV genome copies can be appropriate. In some aspects, a dosage of between about 10¹¹ to 10¹⁵ per kg or appropriate measurement rAAV genome copies can be appropriate. In some aspects, a dosage of about 1×10¹⁴ vector genome (vg) copies per kg or appropriate measurement can be appropriate. In some aspects, the dosage can vary or be reduced when specifically targeting one or more brain region(s). In some aspects, a dosage between about 10⁷ to 10⁸ rAAV genome copies per kg or appropriate measurement can be appropriate. In some aspects, a dosage of between about 10⁸ to 10⁹ rAAV genome copies per kg or appropriate measurement can be appropriate. In some aspects, a dosage of between about 10⁹ to 10¹⁰ rAAV genome copies per kg or appropriate measurement can be appropriate. In some aspects, a dosage of between about 10¹⁰ to 10¹¹ rAAV genome copies per kg or other appropriate measurement can be appropriate.

In some aspects, a potential side-effect for administering an AAV virion to a subject can be an immune response in the subject to the AAV virion, including inflammation, and, and may depend on the route of administration, and in particularly, when the administration of an AAV virion is systemic. In some aspects, a subject can be immunosuppressed prior to administration of one or more rAAVs as described herein.

As used herein, “immunosuppressed” or “immunosuppression” refers to a decrease in the activation or efficacy of an immune response in a subject. Immunosuppression can be induced in a subject using one or more (e.g., multiple, such as 2, 3, 4, 5, or more) agents, including, but not limited to, rituximab, methylprednisolone, prednisolone, sirolimus, immunoglobulin injection, prednisone, methotrexate, an interleukin-6 inhibitor, an anti-interleukin-6 antibody, an interleukin-6 receptor inhibitor, an anti-interleukin-6 receptor antibody, and any combination thereof.

In some aspects, methods disclosed herein can further comprise the step of inducing immunosuppression (e.g., administering one or more immunosuppressive agents) in a subject prior to the subject being administered an rAAV virion (e.g., an rAAV virion or pharmaceutical composition as disclosed herein). In some aspects, a subject can be immunosuppressed (e.g., immunosuppression is induced in the subject) between about 30 days and about 0 days (e.g., any time between 30 days until administration of the rAAV virion, inclusive) prior to administration of the rAAV virion to the subject. In some aspects, the subject can be pre-treated with immune suppression agent (e.g., rituximab, sirolimus, and/or prednisone) for at least 7 days.

In some aspects, immunosuppression of a subject maintained during and/or after administration of a rAAV virion or pharmaceutical composition. In some aspects, a subject can be immunosuppressed (e.g., administered one or more immunosuppressants) for between 1 day and 1 year after administration of the rAAV virion or pharmaceutical composition.

In some aspects, rAAV virion compositions can be formulated to reduce aggregation of AAV virions in the composition, particularly where high rAAV virion concentrations are present (e.g., −10¹³ GC/ml or more). Methods for reducing aggregation of rAAVs include, for example, addition of surfactants, pH adjustment, salt concentration adjustment, etc. (Sec, e.g., Wright F R, et al., Molecular Therapy (2005) 12, 171-178.)

In some aspects, these formulations can contain at least about 0.1% of the active compound or more, although the percentage of the active ingredient(s) may, of course, be varied and can be conveniently be between about 1 or 2% and about 70% or 80% or more of the weight or volume of the total formulation. Naturally, the amount of active compound in each therapeutically-useful composition can be prepared in such a way that a suitable dosage can be obtained in any given unit dose of the compound. Factors such as solubility, bioavailability, biological half-life, route of administration, product shelf life, as well as other pharmacological considerations can be contemplated by one skilled in the art of preparing such pharmaceutical formulations, and as such, a variety of dosages and treatment regimens can be desirable.

In some aspects, it will be desirable to deliver the rAAV virions in suitably formulated pharmaceutical compositions as disclosed herein either subcutaneously, intrapancreatically, intranasally, parenterally, intravenously, intramuscularly, intrathecally, or orally, intraperitoneally, intracerebroventricularly, or by inhalation. In some aspects, the administration modalities as described in U.S. Pat. Nos. 5,543,158; 5,641,515 and 5,399,363 can be used to deliver rAAVs. In some embodiments, a preferred mode of administration can be by intracerebroventricular or intrathecal injection.

The pharmaceutical forms suitable for injectable use include sterile aqueous solutions or dispersions and sterile powders for the extemporaneous preparation of sterile injectable solutions or dispersions. Dispersions can also be prepared in glycerol, liquid polyethylene glycols, and mixtures thereof and in oils. Under ordinary conditions of storage and use, these preparations contain a preservative to prevent the growth of microorganisms. In many cases the form can be sterile and fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, isotonic agents, for example, sugars or sodium chloride can be included. Prolonged absorption of the injectable compositions can be brought about by the use in the compositions of agents delaying absorption, for example, aluminum monostearate and gelatin.

For administration of an injectable aqueous solution, for example, the solution can be suitably buffered, if necessary, and the liquid diluent first rendered isotonic with sufficient saline or glucose. These particular aqueous solutions can be suitable for intravenous, intramuscular, subcutaneous, intracerebroventricular, and intraperitoneal administration. In this connection, a sterile aqueous medium can be employed. For example, one dosage can be dissolved in 1 mL of isotonic NaCl solution and either added to 1000 mL of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the host. The person responsible for administration will, in any event, determine the appropriate dose for the individual host.

Sterile injectable solutions can be prepared by incorporating the active rAAV virion in the required amount in the appropriate solvent with various of the other ingredients enumerated herein, as required, followed by filtered sterilization. Generally, dispersions can be prepared by incorporating the various sterilized active ingredients into a sterile vehicle which contains the basic dispersion medium and the required other ingredients from those enumerated above. In the case of sterile powders for the preparation of sterile injectable solutions, the methods of preparation can be vacuum-drying and freeze-drying techniques which yield a powder of the active ingredient plus any additional desired ingredient from a previously sterile-filtered solution thereof.

The rAAV virion compositions disclosed herein can be also be formulated in a neutral or salt form. Pharmaceutically-acceptable salts, include the acid addition salts (formed with the free amino groups of the protein) and which can be formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine, and the like. Upon formulation, solutions will be administered in a manner compatible with the dosage formulation and in such amount as is therapeutically effective. The formulations can be easily administered in a variety of dosage forms such as injectable solutions, drug-release capsules, and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. Supplementary active ingredients can also be incorporated into the compositions. The phrase “pharmaceutically-acceptable” refers to molecular entities and compositions that do not produce an allergic or similar untoward reaction when administered to a host.

Delivery vehicles such as liposomes, nanocapsules, microparticles, microspheres, lipid particles, vesicles, and the like, may be used for the introduction of the compositions of the present disclosure into suitable host cells. In particular, the rAAV vector delivered transgenes can be formulated for delivery either encapsulated in a lipid particle, a liposome, a vesicle, a nanosphere, or a nanoparticle or the like.

Such formulations can be used for the introduction of pharmaceutically acceptable formulations of the nucleic acids or the rAAV constructs disclosed herein. The formation and use of liposomes is generally known to those of skill in the art. Recently, liposomes were developed with improved serum stability and circulation half-lives (U.S. Pat. No. 5,741,516). Further, various methods of liposome and liposome like preparations as potential drug carriers have been described (U.S. Pat. Nos. 5,567,434; 5,552,157; 5,565,213; 5,738,868 and 5,795,587).

Liposomes have been used successfully with a number of cell types that are normally resistant to transfection by other procedures. In addition, liposomes are free of the DNA length constraints that are typical of viral-based delivery systems. Liposomes have been used effectively to introduce genes, drugs, radiotherapeutic agents, viruses, transcription factors and allosteric effectors into a variety of cultured cell lines and animals. In addition, several successful clinical trials examining the effectiveness of liposome-mediated drug delivery have been completed.

Liposomes can be formed from phospholipids that can be dispersed in an aqueous medium and spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500 Angstroms, containing an aqueous solution in the core.

Alternatively, nanocapsule formulations of the rAAV virions can be used. Nanocapsules can generally entrap substances in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use.

In addition to the methods of delivery described above, the following techniques can also be used as alternative methods of delivering the rAAV compositions to a host.

Sonophoresis (e.g., ultrasound) has been used and described in U.S. Pat. No. 5,656,016 as a device for enhancing the rate and efficacy of drug permeation into and through the circulatory system. Other drug delivery alternatives contemplated are intraosseous injection (U.S. Pat. No. 5,779,708), microchip devices (U.S. Pat. No. 5,797,898), ophthalmic formulations (Bourlais et al., 1998), transdermal matrices (U.S. Pat. Nos. 5,770,219 and 5,783,208) and feedback-controlled delivery (U.S. Pat. No. 5,697,899).

In some aspects, the methods can include administering one or more additional therapeutic agents to a subject who has been administered an rAAV or pharmaceutical composition as described herein.

In some aspects, administering the rAAV virions described to a subject promotes expression of GALT by 10-fold compared to a control. In some aspects, administering the rAAV virions described herein to a subject promotes expression of GALT by 5-fold to 100-fold compared to control (e.g., 5-fold to 10-fold, 10-fold to 15-fold, 10-fold to 20-fold, 15-fold to 25-fold, 20-fold to 30-fold, 25-fold to 35-fold, 30-fold to 40-fold, 35-fold to 45-fold, 40-fold to 60-fold, 50-fold to 75-fold, 60-fold to 80-fold, 75-fold to 100-fold compared to a control).

In some aspects, administering the rAAV virions described herein to a subject promotes expression of GALT in a subject (e.g., promotes expression of GALT in the CNS of a subject) by between a 5% and 200% increase (e.g., 5-50%, 25-75%, 50-100%, 75-125%, 100-200%, or 100-150% etc.) compared to a control subject.

As used herein, the term “treating” refers to the application or administration of a composition (e.g., an isolated nucleic acid or rAAV as described herein) to a subject who has a disease or disorder associated with low levels of GALT expression (e.g., GALT deficiency), with the purpose to cure, heal, alleviate, relieve, alter, remedy, ameliorate, improve, or affect the disorder, the symptom of the disease, or the predisposition toward a disease.

Alleviating a disease associated with low levels of GALT expression (e.g., GALT deficiency) includes delaying the development or progression of the disease, or reducing disease severity. Alleviating the disease does not necessarily require curative results. As used therein, “delaying” the development of a disease means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

In particular, administration of the rAAV virion described herein to a human subject suffering from GALT deficiency will within 10 weeks, 15 weeks, 20 weeks, 25 weeks, 30 weeks, 40 weeks, 50 weeks or 1 year after the administration will result in reduction in one or more biomarkers or hallmarks of the disease.

“Development” or “progression” of a disease means initial manifestations and/or ensuing progression of the disease. Development of the disease can be detectable and assessed using standard clinical techniques as well known in the art. However, development also refers to progression that can be undetectable. As used herein the terms development or progression refer to the biological course of the symptoms. “Development” includes occurrence, recurrence, and onset. As used herein “onset” or “occurrence” of a disease can be associated with low levels of GALT expression (e.g., GALT deficiency).

In some aspects, a subject has or is suspected of having a disease or disorder associated with low levels of GALT expression (e.g., GALT deficiency). In some aspects, a subject having a disease or disorder associated with low levels of GALT expression (e.g., GALT deficiency) comprises at least one GALT allele having a loss-of-function mutation (e.g., associated with GALT deficiency). In some aspects, a GALT allele having a loss-of-function mutation (e.g., associated with GALT deficiency) comprises a frameshift mutation, a splice site mutation, a missense mutation, a truncation mutation or a nonsense mutation. A subject may have two GALT alleles having the same loss-of-function mutations (homozygous state) or two GALT alleles having different loss-of-function mutations (compound heterozygous state). In certain aspects, the subject is a carrier of an GALT deficiency and, in certain aspects, is heterozygous for a loss of function allele described herein.

In some aspects, the rAAV virions disclosed herein can be administered in sufficient amounts to transfect the cells of a desired tissue and to provide sufficient levels of gene transfer and expression without undue adverse effects. Pharmaceutically acceptable routes of administration include, but are not limited to, direct delivery to the selected organ (e.g., to the central nervous system), by ICV or administration to the cisterna magna, oral, inhalation (including intranasal and intratracheal delivery), intraocular, intracerebroventricular, intravenous, intramuscular, subcutaneous, intradermal, and other parental routes of administration. Routes of administration can be combined, if desired.

### Kits

Disclosed herein are kits comprising any of the agents described herein. In some aspects, any of the agents disclosed herein can be assembled into pharmaceutical or diagnostic or research kits to facilitate their use in therapeutic, diagnostic or research applications. A kit can include one or more containers housing the components of the disclosure and instructions for use. Specifically, such kits may include one or more agents described herein, along with instructions describing the intended application and the proper use of these agents. In some aspects, the agents in a kit can be in a pharmaceutical formulation and dosage suitable for a particular application and for a method of administration of the agents. Kits for research purposes can contain the components in appropriate concentrations or quantities for running various experiments.

Also disclosed herein are kits for producing a rAAV virions. In some aspects, the kit can comprise a container housing an isolated nucleic acid encoding a GALT1 protein or a portion thereof. In some aspects, the kits can further comprise instructions for producing the rAAV virion. In some aspects, the kit further comprises at least one container housing a recombinant AAV vector, wherein the recombinant AAV vector comprises a transgene (i.e GALT).

In some aspects, the kits can comprise a container housing a recombinant AAV virion as described supra. In some aspects, the kits can further comprises a container housing a pharmaceutically acceptable carrier. For example, a kit can comprise one container housing a rAAV virion and a second container housing a buffer suitable for injection of the rAAV virion into a subject. In some aspects, the container can be a syringe.

In some aspects, the kits can be designed to facilitate use of the methods described herein by researchers and can take many forms. Each of the compositions of the kit, where applicable, may be provided in liquid form (e.g., in solution), or in solid form, (e.g., a dry powder). In some aspects, some of the compositions can be constitutable or otherwise processable (e.g., to an active form), for example, by the addition of a suitable solvent or other species (for example, water or a cell culture medium), which may or may not be provided with the kit. As used herein, “instructions” can define a component of instruction and/or promotion, and typically involve written instructions on or associated with packaging of the disclosure. Instructions also can include any oral or electronic instructions provided in any manner such that a user will clearly recognize that the instructions can be associated with the kit, for example, audiovisual (e.g., videotape, DVD, etc.), internet, and/or web-based communications, etc. The written instructions can be in a form prescribed by a governmental agency regulating the manufacture, use or sale of pharmaceuticals or biological products, which instructions can also reflect approval by the agency of manufacture, use or sale for animal administration.

The kits disclosed herein can also contain any one or more of the components described herein in one or more containers. In some aspects, the kits can include instructions for mixing one or more components of the kit and/or isolating and mixing a sample and applying to a subject. The kits can include a container housing agents described herein. The agents can be in the form of a liquid, gel or solid (powder). The agents can be prepared sterilely, packaged in syringe and shipped refrigerated. Alternatively, it can be housed in a vial or other container for storage. A second container can have other agents prepared sterilely. Alternatively the kits can include the active agents premixed and shipped in a syringe, vial, tube, or other container. The kits can have one or more or all of the components required to administer the agents to an animal, such as a syringe, topical application devices, or iv needle tubing and bag, particularly in the case of the kits for producing specific somatic animal models.

In some aspects, the method disclosed herein can involve transfecting cells with total cellular DNAs isolated from the tissues that potentially harbor proviral AAV genomes at very low abundance and supplementing with helper virus function (e.g., adenovirus) to trigger and/or boost AAV rep and cap gene transcription in the transfected cell. In some aspects, RNA from the transfected cells can provide a template for RT-PCR amplification of cDNA and the detection of novel AAVs. In cases where cells are transfected with total cellular DNAs isolated from the tissues that potentially harbor proviral AAV genomes, it is often desirable to supplement the cells with factors that promote AAV gene transcription. For example, the cells can also be infected with a helper virus, such as an Adenovirus or a Herpes Virus. In some aspects, the helper functions can be provided by an adenovirus. The adenovirus can be a wild-type adenovirus, and can be of human or non-human origin, for example, non-human primate (NHP) origin. Similarly, adenoviruses known to infect non-human animals (e.g., chimpanzees, mouse) can also be employed in the methods of the disclosure (Scc, e.g., U.S. Pat. No. 6,083,716). In addition to wild-type adenoviruses, recombinant viruses or non-viral vectors (e.g., plasmids, episomes, etc.) carrying the necessary helper functions can be utilized. Such recombinant viruses are known in the art and may be prepared according to published techniques. Sec, e.g., U.S. Pat. Nos. 5,871,982 and 6,251,677, which describe a hybrid Ad/AAV virus. A variety of adenovirus strains are available from the American Type Culture Collection, Manassas, Va., or available by request from a variety of commercial and institutional sources. Further, the sequences of many such strains are available from a variety of databases including, e.g., PubMed and GenBank.

Cells can also be transfected with a vector (e.g., helper vector) which provides helper functions to the AAV. The vector providing helper functions can provide adenovirus functions, including. e.g., E1a, E1b, E2a, E4ORF6. The sequences of adenovirus gene providing these functions can be obtained from any known adenovirus serotype, such as serotypes 2, 3, 4, 7, 12 and 40, and further including any of the presently identified human types known in the art. Thus, in some aspects, the methods involve transfecting the cell with a vector expressing one or more genes necessary for AAV replication, AAV gene transcription, and/or AAV packaging.

In some aspects, an isolated capsid gene can be used to construct and package recombinant AAV vectors, using methods well known in the art, to determine functional characteristics associated with the novel capsid protein encoded by the gene. For example, isolated capsid genes can be used to construct and package recombinant AAV (rAAV) vectors comprising a reporter gene (e.g., B-Galactosidase, GFP, Luciferase, etc.). The rAAV vector can then be delivered to an animal (e.g., mouse) and the tissue targeting properties of the isolated capsid gene can be determined by examining the expression of the reporter gene in various tissues (e.g., heart, liver, kidneys) of the animal. Other methods for characterizing isolated capsid genes are disclosed herein and still others are well known in the art.

The kits disclosed can have a variety of forms, such as a blister pouch, a shrink wrapped pouch, a vacuum scalable pouch, a scalable thermoformed tray, or a similar pouch or tray form, with the accessories loosely packed within the pouch, one or more tubes, containers, a box or a bag. The kits can be sterilized after the accessories are added, thereby allowing the individual accessories in the container to be otherwise unwrapped. The kits can be sterilized using any appropriate sterilization techniques, such as radiation sterilization, heat sterilization, or other sterilization methods known in the art. The kits can also include other components, depending on the specific application, for example, containers, cell media, salts, buffers, reagents, syringes, needles, a fabric, such as gauze, for applying or removing a disinfecting agent, disposable gloves, a support for the agents prior to administration etc.

The instructions included within the kit can involve methods for detecting a latent AAV in a cell. In addition, kits of the disclosure can include, instructions, a negative and/or positive control, containers, diluents and buffers for the sample, sample preparation tubes and a printed or electronic table of reference AAV sequence for sequence comparisons.

TABLE 1
Sequences

| | SEQ ID | |
|---|---|---|
| Name | NO: | Sequence |
| hGALT | 1 | MSRSGTDPQQRQQASEADAAAATFRANDHQHIRYNPLQDEWVLVSAHR |
| protein | | MKRPWQGQVEPQLLKTVPRHDPLNPLCPGAIRANGEVNPQYDSTFLFD |
| | | NDFPALQPDAPSPGPSDHPLFQAKSARGVCKVMCFHPWSDVTLPLMSV |
| | | PEIRAVVDAWASVTEELGAQYPWVQIFENKGAMMGCSNPHPHCQVWAS |
| | | SFLPDIAQREERSQQAYKSQHGEPLLMEYSRQELLRKERLVLTSEHWL |
| | | VLVPFWATWPYQTLLLPRRHVRRLPELTPAERDDLASIMKKLLTKYDN |
| | | LFETSFPYSMGWHGAPTGSEAGANWNHWQLHAHYYPPLLRSATVRKFM |
| | | VGYEMLAQAQRDLTPEQAAERLRALPEVHYHLGQKDRETATIA |
| Wild type | 2 | atgagcagaagcggcaccgaccctcagcagagacagcaggcctctgaa |
| hGALT | | gccgatgccgccgctgccaccttcagagccaatgaccaccagcacatc |
| coding | | cggtacaaccccctgcaggacgagtgggtgctggtgtccgcccacaga |
| sequence | | atgaagaggccttggcagggccaggtggaaccccagctgctgaaaacc |
| | | gtgcccagacacgaccccctgaaccctctgtgtcctggcgccattaga |
| | | gccaacggcgaagtgaacccccagtacgacagcaccttcctgttcgac |
| | | aacgacttccccgccctgcagcctgatgccccatctcctggacctagc |
| | | gaccaccctctgttccaggccaagtctgccagaggcgtgtgcaaagtg |
| | | atgtgcttccacccttggagcgacgtgaccctgcccctgatgagcgtg |
| | | ccagagatcagagccgtggtggatgcctgggccagcgtgacagaagaa |
| | | ctgggagcccagtacccctgggtgcagatcttcgagaacaagggcgcc |
| | | atgatgggctgcagcaacccccaccctcactgtcaagtgtgggccagc |
| | | agcttcctgcccgatatcgcccagcgggaagagagaagccagcaggct |
| | | tacaagagccagcacggcgagcccctgctgatggaatactccagacag |
| | | gaactgctgcggaaagaacggctggtgctgaccagcgagcactggctg |
| | | gtgctggtgcctttttgggccacatggccctaccagaccctgctgctg |
| | | cctagaaggcacgtgcggagactgcctgagctgacacccgccgagaga |
| | | gatgacctggccagcatcatgaagaaactgctgaccaaatacgacaac |
| | | ctgttcgagaccagcttcccctacagcatgggctggcacggcgctcct |
| | | acaggatctgaggctggcgccaactggaaccactggcagctgcacgcc |
| | | cactactaccccccactgctgagatctgccaccgtgcggaagttcatg |
| | | gtgggatacgagatgctggctcaggcccagagagatctgacccctgaa |
| | | caggccgccgaacggctgagagcactgcccgaagtgcactaccacctg |
| | | ggacagaaggacagagagacagccacaatcgcctga |
| pscAAV- | 3 | ttggccactccctctctgcgcgctcgctcgctcactgaggccgcccgg |
| CAG- | | gcaaagcccgggcgtcgggcgacctttggtcgcccggcctcagtgagc |
| hGALT | | gagcgagcgcgcagagagggagtggccaactccatcactaggggttcc |
| expression | | tgatgtaatacgactcactagttcctggaggggtggagtcgtgaccta |
| cassette | | ggccattgacgtcaataatgacgtatgttcccatagtaacgccaatag |
| (including | | ggactttccattgacgtcaatgggtggagtatttacggtaaactgccc |
| flanking | | acttggcagtacatcaagtgtatcatatgccaagtacgccccctattg |
| ITRs) | | acgtcaatgacggtaaatggcccgcctggcattatgcccagtacatga |
| | | ccttatgggactttcctacttggcagtacatctacgtattagtcatcg |
| | | ctattaccatggtcgaggtgagccccacgttctgcttcactctcccca |
| | | tctcccccccctccccacccccaattttgtatttatttattttttaat |
| | | tattttgtgcagcgatgggggcggggggggggggggggcgcgcgccag |
| | | gcggggcggggcggggcgaggggcggggcggggcgaggcggagaggtg |
| | | cggcggcagccaatcagagcggcgcgctccgaaagtttccttttatgg |
| | | cgaggcggcggcggcggcggccctataaaaagcgaagcgcgcggcggg |
| | | cgggagtcgctgcgcgctgccttcgccccgtgccccgctccgccgccg |
| | | cctcgcgccgcccgccccggctctgactgaccgcgttactcccacagg |
| | | tgagcgggcgggacggcccttctcctccgggctgtaattagcgcttgg |
| | | gaattcgccaccatgagcagaagcggcaccgaccctcagcagagacag |
| | | caggcctctgaagccgatgccgccgctgccaccttcagagccaatgac |
| | | caccagcacatccggtacaaccccctgcaggacgagtgggtgctggtg |
| | | tccgcccacagaatgaagaggccttggcagggccaggtggaaccccag |
| | | ctgctgaaaaccgtgcccagacacgaccccctgaaccctctgtgtcct |
| | | ggcgccattagagccaacggcgaagtgaacccccagtacgacagcacc |
| | | ttcctgttcgacaacgacttccccgccctgcagcctgatgccccatct |
| | | cctggacctagcgaccaccctctgttccaggccaagtctgccagaggc |
| | | gtgtgcaaagtgatgtgcttccacccttggagcgacgtgaccctgccc |
| | | ctgatgagcgtgccagagatcagagccgtggtggatgcctgggccagc |
| | | gtgacagaagaactgggagcccagtacccctgggtgcagatcttcgag |
| | | aacaagggcgccatgatgggctgcagcaacccccaccctcactgtcaa |
| | | gtgtgggccagcagcttcctgcccgatatcgcccagcgggaagagaga |
| | | agccagcaggcttacaagagccagcacggcgagcccctgctgatggaa |
| | | tactccagacaggaactgctgcggaaagaacggctggtgctgaccagc |
| | | gagcactggctggtgctggtgcctttttgggccacatggccctaccag |
| | | accctgctgctgcctagaaggcacgtgcggagactgcctgagctgaca |
| | | cccgccgagagagatgacctggccagcatcatgaagaaactgctgacc |
| | | aaatacgacaacctgttcgagaccagcttcccctacagcatgggctgg |
| | | cacggcgctcctacaggatctgaggctggcgccaactggaaccactgg |
| | | cagctgcacgcccactactaccccccactgctgagatctgccaccgtg |
| | | cggaagttcatggtgggatacgagatgctggctcaggcccagagagat |
| | | ctgacccctgaacaggccgccgaacggctgagagcactgcccgaagtg |
| | | cactaccacctgggacagaaggacagagagacagccacaatcgcctga |
| | | agcgctgctcgagagatctacaacttgtttattgcagcttataatggt |
| | | tacaaataaagcaatagcatcacaaatttcacaaataaagcatttttt |
| | | tcactgcattctagttgtggtttgtccaaactcatcaatgtatcttaa |
| | | ggcctaggtgagctctggtaccctctccactccctctctgcgcgctcg |
| | | ctcgctcactgaggccgggcgaccaaaggtcgcccgacgcccgggctt |
| | | tgcccgggcggcctcagtgagcgagcgagcgcgcag |
| pAAV2ITR- | 4 | ttggccactccctctctgcgcgctcgctcgctcactgaggccgcccgg |
| CAG- | | gcaaagcccgggcgtcgggcgacctttggtcgcccggcctcagtgagc |
| hGALT | | gagcgagcgcgcagagagggagtggccaactccatcactaggggttcc |
| expression | | tgatgtaatacgactcactagttattaatagtaatcaattacggggtc |
| cassette | | attagttcatagcccatatatggagttccgcgttacataacttacggt |
| (including | | aaatggcccgcctggctgaccgcccaacgacccccgcccattgacgtc |
| flanking | | aataatgacgtatgttcccatagtaacgccaatagggactttccattg |
| ITRs) | | acgtcaatgggtggactatttacggtaaactgcccacttggcagtaca |
| | | tcaagtgtatcatatgccaagtacgccccctattgacgtcaatgacgg |
| | | taaatggcccgcctggcattatgcccagtacatgaccttatgggactt |
| | | tcctacttggcagtacatctacgtattagtcatcgctattaccatggg |
| | | tcgaggtgagccccacgttctgcttcactctccccatctcccccccct |
| | | ccccacccccaattttgtatttatttattttttaattattttgtgcag |
| | | cgatgggggcggggggggggggggcgcgcgccaggcggggcggggcgg |
| | | ggcgaggggcggggcggggcgaggcggagaggtgcggcggcagccaat |
| | | cagagcggcgcgctccgaaagtttccttttatggcgaggcggcggcgg |
| | | cggcggccctataaaaagcgaagcgcgcggcgggcgggagtcgctgcg |
| | | ttgccttcgccccgtgccccgctccgcgccgcctcgcgccgcccgccc |
| | | cggctctgactgaccgcgttactcccacaggtgagcgggcgggacggc |
| | | ccttctcctccgggctgtaattagcgcttggtttaatgacggctcgtt |
| | | tcttttctgtggctgcgtgaaagccttaaagggctccgggagggccct |
| | | ttgtgcgggggggagcggctcggggggtgcgtgcgtgtgtgtgtgcgt |
| | | ggggagcgccgcgtgcggcccgcgctgcccggcggctgtgagcgctgc |
| | | gggcgcggcgcggggctttgtgcgctccgcgtgtgcgcgaggggagcg |
| | | cggccgggggcggtgccccgcggtgcgggggggctgcgaggggaacaa |
| | | aggctgcgtgcggggtgtgtgcgtgggggggtgagcagggggtgtggg |
| | | cgcggcggtcgggctgtaacccccccctgcacccccctccccgagttg |
| | | ctgagcacggcccggcttcgggtgcggggctccgtgcggggcgtggcg |
| | | cggggctcgccgtgccgggcggggggtggcggcaggtgggggtgccgg |
| | | gcggggcggggccgcctcgggccggggagggctcgggggaggggcgcg |
| | | gcggccccggagcgccggcggctgtcgaggcgcggcgagccgcagcca |
| | | ttgccttttatggtaatcgtgcgagagggcgcagggacttcctttgtc |
| | | ccaaatctggcggagccgaaatctgggaggcgccgccgcaccccctct |
| | | agcgggcgcgggcgaagcggtgcggcgccggcaggaaggaaatgggcg |
| | | gggagggccttcgtgcgtcgccgcgccgccgtccccttctccatctcc |
| | | agcctcggggctgccgcagggggacggctgccttcgggggggacgggg |
| | | cagggcggggttcggcttctggcgtgtgaccggcggctctagagcctc |
| | | tgctaaccatgttcatgccttcttctttttcctacaggccaccatgtc |
| | | gcgcagtggaaccgatcctcagcaacgccagcaggcgtcagaggcgga |
| | | cgccgcagcagcaaccttccgggcaaacgaccatcagcatatccgcta |
| | | caacccgctgcaggatgagtgggtgctggtgtcagctcaccgcatgaa |
| | | gcggccctggcagggtcaagtggagccccagcttctgaagacagtgcc |
| | | ccgccatgaccctctcaaccctctgtgtcctggggccatccgagccaa |
| | | cggagaggtgaatccccagtacgatagcaccttcctgtttgacaacga |
| | | cttcccagctctgcagcctgatgcccccagtccaggacccagtgatca |
| | | tccccttttccaagcaaagtctgctcgaggagtctgtaaggtcatgtg |
| | | cttccacccctggtcggatgtaacgctgccactcatgtcggtccctga |
| | | gatccgggctgttgttgatgcatgggcctcagtcacagaggagctggg |
| | | tgcccagtacccttgggtgcagatctttgaaaacaaaggtgccatgat |
| | | gggctgttctaacccccacccccactgccaggtatgggccagcagttt |
| | | cctgccagatattgcccagcgtgaggagcgatctcagcaggcctataa |
| | | gagtcagcatggagagcccctgctaatggagtacagccgccaggagct |
| | | actcaggaaggaacgtctggtcctaaccagtgagcactggttagtact |
| | | ggtccccttctgggcaacatggccctaccagacactgctgctgccccg |
| | | tcggcatgtgcggcggctacctgagctgacccctgctgagcgtgatga |
| | | tctagcctccatcatgaagaagctcttgaccaagtatgacaacctctt |
| | | tgagacgtcctttccctactccatgggctggcatggggctcccacagg |
| | | atcagaggctggggccaactggaaccattggcagctgcacgctcatta |
| | | ctaccctccgctcctgcgctctgccactgtccggaaattcatggttgg |
| | | ctacgaaatgcttgctcaggctcagagggacctcacccctgagcaggc |
| | | tgcagagagactaagggcacttcctgaggttcattaccacctggggca |
| | | gaaggacagggagacagcaaccatcgcctga |
| pAAV2ITR- | 5 | ttggccactccctctctgcgcgctcgctcgctcactgaggccgcccgggcaaagcccgggcgtcgg |
| EF1a- | | gcgacctttggtcgcccggcctcagtgagcgagcgagcgcgcagagagggagtggccaactccat |
| hGALT | | cactaggggttcctgatgtaatacgactcactagtgctccggtgcccg |
| expression | | tcagtgggcagagcgcacatcgcccacagtccccgagaagttgggggg |
| cassette | | aggggtcggcaattgaaccggtgcctagagaaggtggcgcggggtaaa |
| (including | | ctgggaaagtgatgtcgtgtactggctccgcctttttcccgagggtgg |
| flanking | | gggagaaccgtatataagtgcagtagtcgccgtgaacgttctttttcg |
| ITRs) | | caacgggtttgccgccagaacacaggtaagtgccgtgtgtggttcccg |
| | | cgggcctggcctctttacgggttatggcccttgcgtgccttgaattac |
| | | ttccacgcccctggctgcagtacgtgattcttgatcccgagcttcggg |
| | | ttggaagtgggtgggagagttcgaggccttgcgcttaaggagcccctt |
| | | cgcctcgtgcttgagttgaggcctggcctgggcgctggggccgccgcg |
| | | tgcgaatctggtggcaccttcgcgcctgtctcgctgctttcgataagt |
| | | ctctagccatttaaaatttttgatgacctgctgcgacgctttttttct |
| | | ggcaagatagtcttgtaaatgcgggccaagatctgcacactggtattt |
| | | cggtttttggggccgcgggcggcgacggggcccgtgcgtcccagcgca |
| | | catgttcggcgaggcggggcctgcgagcgcggccaccgagaatcggac |
| | | gggggtagtctcaagctggccggcctgctctggtgcctggcctcgcgc |
| | | cgccgtgtatcgccccgccctgggcggcaaggctggcccggtcggcac |
| | | cagttgcgtgagcggaaagatggccgcttcccggccctgctgcaggga |
| | | gctcaaaatggaggacgcggcgctcgggagagcgggcgggtgagtcac |
| | | ccacacaaaggaaaagggcctttccgtcctcagccgtcgcttcatgtg |
| | | actccacggagtaccgggcgccgtccaggcacctcgattagttctcga |
| | | gcttttggagtacgtcgtctttaggttggggggaggggttttatgcga |
| | | tggagtttccccacactgagtgggtggagactgaagttaggccagctt |
| | | ggcacttgatgtaattctccttggaatttgccctttttgagtttggat |
| | | cttggttcattctcaagcctcagacagtggttcaaagtttttttcttc |
| | | catttcaggtgtcgtgagccaccatgtcgcgcagtggaaccgatcctc |
| | | agcaacgccagcaggcgtcagaggcggacgccgcagcagcaaccttcc |
| | | gggcaaacgaccatcagcatatccgctacaacccgctgcaggatgagt |
| | | gggtgctggtgtcagctcaccgcatgaagcggccctggcagggtcaag |
| | | tggagccccagcttctgaagacagtgccccgccatgaccctctcaacc |
| | | ctctgtgtcctggggccatccgagccaacggagaggtgaatccccagt |
| | | acgatagcaccttcctgtttgacaacgacttcccagctctgcagcctg |
| | | atgcccccagtccaggacccagtgatcatccccttttccaagcaaagt |
| | | ctgctcgaggagtctgtaaggtcatgtgcttccacccctggtcggatg |
| | | taacgctgccactcatgtcggtccctgagatccgggctgttgttgatg |
| | | catgggcctcagtcacagaggagctgggtgcccagtacccttgggtgc |
| | | agatctttgaaaacaaaggtgccatgatgggctgttctaacccccacc |
| | | cccactgccaggtatgggccagcagtttcctgccagatattgcccagc |
| | | gtgaggagcgatctcagcaggcctataagagtcagcatggagagcccc |
| | | tgctaatggagtacagccgccaggagctactcaggaaggaacgtctgg |
| | | tcctaaccagtgagcactggttagtactggtccccttctgggcaacat |
| | | ggccctaccagacactgctgctgccccgtcggcatgtgcggcggctac |
| | | ctgagctgacccctgctgagcgtgatgatctagcctccatcatgaaga |
| | | agctcttgaccaagtatgacaacctctttgagacgtcctttccctact |
| | | ccatgggctggcatggggctcccacaggatcagaggctggggccaact |
| | | ggaaccattggcagctgcacgctcattactaccctccgctcctgcgct |
| | | ctgccactgtccggaaattcatggttggctacgaaatgcttgctcagg |
| | | ctcagagggacctcacccctgagcaggctgcagagagactaagggcac |
| | | ttcctgaggttcattaccacctggggcagaaggacagggagacagcaa |
| | | ccatcgcctgacggcgcgccaagcttatcgataatcaacctctggatt |
| | | acaaaatttgtgaaagattgactggtattcttaactatgttgctcctt |
| | | ttacgctatgtggatacgctgctttaatgcctttgtatcatgctattg |
| | | cttcccgtatggctttcattttctcctccttgtataaatcctggtagc |
| | | tgtctctttatgaggagttgtggcccgttgtcaggcaacgtggcgtag |
| | | tgtgcactgtgtttgctgacgcaacccccactggttggggcattgcca |
| | | ccacctgtcagctcctttccgggactttcgctttccccctccctattg |
| | | ccacggcggaactcatcgccgcctgccttgcccgctgctggacagggg |
| | | ctcggctgttgggcactgacaattccgtggtgttgtcggggaaatcat |
| | | cgtcctttccttggctgctcgcctgtgttgccacctggattctgcgcg |
| | | ggacgtccttctgctacgtcccttcggccctcaatccagcggaccttc |
| | | cttcccgcggcctgctgccggctctgcggcctcttccgcgtcttcgcc |
| | | ttcgccctcagacgagtcggatctccctttgggccgcctccccgcatc |
| | | gataccgagcgctgctcgagagatctacctgtgccttctagttgccag |
| | | ccatctgttgtttgcccctcccccgtgccttccttgaccctggaaggt |
| | | gccactcccactgtcctttcctaataaaatgaggaaattgcatcgcat |
| | | tgtctgagtaggtgtcattctattctggggggtggggtggggcaggac |
| | | agcaagggggaggattgggaagacaatagcaggcatgctggggatgcg |
| | | gtgggctctatggaggcctaggtgagctctggtaccctctagtcaagg |
| | | atcaggaacccctagtgatggagttggccactccctctctgcgcgctc |
| | | gctcgctcactgaggccgggcgaccaaaggtcgcccgacgcccgggct |
| | | ttgcccgggcggcctcagtgagcgagcgagcgcgcagagagggagtgg |
| | | ccaa |
| pscAAV- | 6 | tcgcgcgtttcggtgatgacggtgaaaacctctgacacatgcagctcc |
| CAG- | | cggagacggtcacagcttgtctgtaagcggatgccgggagcagacaag |
| hGALT | | cccgtcagggcgcgtcagcgggtgttggcgggtgtcggggctggctta |
| plasmid | | actatgcggcatcagagcagattgtactgagagtgcaccatatgcggt |
| | | gtgaaataccgcacagatgcgtaaggagaaaataccgcatcaggcgcc |
| | | attcgccattcaggctgcgcaactgttgggaagggcgatcggtgcggg |
| | | cctcttcgctattacgccagctggcgaaagggggatgtgctgcaaggc |
| | | gattaagttgggtaacgccagggttttcccagtcacgacgttgtaaaa |
| | | cgacggccagtaggcgcgcccaaaggctatcaggtcattgcctgagag |
| | | tctggagcaaacaagagaatcgttggccactccctctctgcgcgctcg |
| | | ctcgctcactgaggccgcccgggcaaagcccgggcgtcgggcgacctt |
| | | tggtcgcccggcctcagtgagcgagcgagcgcgcagagagggagtggc |
| | | caactccatcactaggggttcctgatgtaatacgactcactagttcct |
| | | ggaggggtggagtcgtgacctaggccattgacgtcaataatgacgtat |
| | | gttcccatagtaacgccaatagggactttccattgacgtcaatgggtg |
| | | gagtatttacggtaaactgcccacttggcagtacatcaagtgtatcat |
| | | atgccaagtacgccccctattgacgtcaatgacggtaaatggcccgcc |
| | | tggcattatgcccagtacatgaccttatgggactttcctacttggcag |
| | | tacatctacgtattagtcatcgctattaccatggtcgaggtgagcccc |
| | | acgttctgcttcactctccccatctcccccccctccccacccccaatt |
| | | ttgtatttatttattttttaattattttgtgcagcgatgggggcgggg |
| | | ggggggggggggcgcgcgccaggcggggcggggcggggcgaggggcgg |
| | | ggcggggcgaggcggagaggtgcggcggcagccaatcagagcggcgcg |
| | | ctccgaaagtttccttttatggcgaggcggcggcggcggcggccctat |
| | | aaaaagcgaagcgcgcggcgggcgggagtcgctgcgcgctgccttcgc |
| | | cccgtgccccgctccgccgccgcctcgcgccgcccgccccggctctga |
| | | ctgaccgcgttactcccacaggtgagcgggcgggacggcccttctcct |
| | | ccgggctgtaattagcgcttgggaattcgccaccatgagcagaagcgg |
| | | caccgaccctcagcagagacagcaggcctctgaagccgatgccgccgc |
| | | tgccaccttcagagccaatgaccaccagcacatccggtacaaccccct |
| | | gcaggacgagtgggtgctggtgtccgcccacagaatgaagaggccttg |
| | | gcagggccaggtggaaccccagctgctgaaaaccgtgcccagacacga |
| | | ccccctgaaccctctgtgtcctggcgccattagagccaacggcgaagt |
| | | gaacccccagtacgacagcaccttcctgttcgacaacgacttccccgc |
| | | cctgcagcctgatgccccatctcctggacctagcgaccaccctctgtt |
| | | ccaggccaagtctgccagaggcgtgtgcaaagtgatgtgcttccaccc |
| | | ttggagcgacgtgaccctgcccctgatgagcgtgccagagatcagagc |
| | | cgtggtggatgcctgggccagcgtgacagaagaactgggagcccagta |
| | | cccctgggtgcagatcttcgagaacaagggcgccatgatgggctgcag |
| | | caacccccaccctcactgtcaagtgtgggccagcagcttcctgcccga |
| | | tatcgcccagcgggaagagagaagccagcaggcttacaagagccagca |
| | | cggcgagcccctgctgatggaatactccagacaggaactgctgcggaa |
| | | agaacggctggtgctgaccagcgagcactggctggtgctggtgccttt |
| | | ttgggccacatggccctaccagaccctgctgctgcctagaaggcacgt |
| | | gcggagactgcctgagctgacacccgccgagagagatgacctggccag |
| | | catcatgaagaaactgctgaccaaatacgacaacctgttcgagaccag |
| | | cttcccctacagcatgggctggcacggcgctcctacaggatctgaggc |
| | | tggcgccaactggaaccactggcagctgcacgcccactactacccccc |
| | | actgctgagatctgccaccgtgcggaagttcatggtgggatacgagat |
| | | gctggctcaggcccagagagatctgacccctgaacaggccgccgaacg |
| | | gctgagagcactgcccgaagtgcactaccacctgggacagaaggacag |
| | | agagacagccacaatcgcctgaagcgctgctcgagagatctacaactt |
| | | gtttattgcagcttataatggttacaaataaagcaatagcatcacaaa |
| | | tttcacaaataaagcatttttttcactgcattctagttgtggtttgtc |
| | | caaactcatcaatgtatcttaaggcctaggtgagctctggtaccctct |
| | | ccactccctctctgcgcgctcgctcgctcactgaggccgggcgaccaa |
| | | aggtcgcccgacgcccgggctttgcccgggcggcctcagtgagcgagc |
| | | gagcgcgcagctgcattaatgaatcggccaacgcgcggggagaggcgg |
| | | tttgcgtattggttaattaacatggtcatagctgtttcctgtgtgaaa |
| | | ttgttatccgctcacaattccacacaacatacgagccggaagcataaa |
| | | gtgtaaagcctggggtgcctaatgagtgagctaactcacattaattgc |
| | | gttgcgctcactgcccgctttccagtcgggaaacctgtcgtgccagct |
| | | gcattaatgaatcggccaacgcgcggggagaggcggtttgcgtattgg |
| | | gcgctcttccgcttcctcgctcactgactcgctgcgctcggtcgttcg |
| | | gctgcggcgagcggtatcagctcactcaaaggcggtaatacggttatc |
| | | cacagaatcaggggataacgcaggaaagaacatgtgagcaaaaggcca |
| | | gcaaaaggccaggaaccgtaaaaaggccgcgttgctggcgtttttcca |
| | | taggctccgcccccctgacgagcatcacaaaaatcgacgctcaagtca |
| | | gaggtggcgaaacccgacaggactataaagataccaggcgtttccccc |
| | | tggaagctccctcgtgcgctctcctgttccgaccctgccgcttaccgg |
| | | atacctgtccgcctttctcccttcgggaagcgtggcgctttctcatag |
| | | ctcacgctgtaggtatctcagttcggtgtaggtcgttcgctccaagct |
| | | gggctgtgtgcacgaaccccccgttcagcccgaccgctgcgccttatc |
| | | cggtaactatcgtcttgagtccaacccggtaagacacgacttatcgcc |
| | | actggcagcagccactggtaacaggattagcagagcgaggtatgtagg |
| | | cggtgctacagagttcttgaagtggtggcctaactacggctacactag |
| | | aagaacagtatttggtatctgcgctctgctgaagccagttaccttcgg |
| | | aaaaagagttggtagctcttgatccggcaaacaaaccaccgctggtag |
| | | cggtggtttttttgtttgcaagcagcagattacgcgcagaaaaaaagg |
| | | atctcaagaagatcctttgatcttttctacggggtctgacgctcagtg |
| | | gaacgaaaactcacgttaagggattttggtcatgagattatcaaaaag |
| | | gatcttcacctagatccttttaaattaaaaatgaagttttaaatcaat |
| | | ctaaagtatatatgagtaaacttggtctgacagttagaaaaactcatc |
| | | gagcatcaaatgaaactgcaatttattcatatcaggattatcaatacc |
| | | atatttttgaaaaagccgtttctgtaatgaaggagaaaactcaccgag |
| | | gcagttccataggatggcaagatcctggtatcggtctgcgattccgac |
| | | tcgtccaacatcaatacaacctattaatttcccctcgtcaaaaataag |
| | | gttatcaagtgagaaatcaccatgagtgacgactgaatccggtgagaa |
| | | tggcaaaagtttatgcatttctttccagacttgttcaacaggccagcc |
| | | attacgctcgtcatcaaaatcactcgcatcaaccaaaccgttattcat |
| | | tcgtgattgcgcctgagcgagacgaaatacgcgatcgctgttaaaagg |
| | | acaattacaaacaggaatcgaatgcaaccggcgcaggaacactgccag |
| | | cgcatcaacaatattttcacctgaatcaggatattcttctaatacctg |
| | | gaatgctgtttttccggggatcgcagtggtgagtaaccatgcatcatc |
| | | aggagtacggataaaatgcttgatggtcggaagaggcataaattccgt |
| | | cagccagtttagtctgaccatctcatctgtaacatcattggcaacgct |
| | | acctttgccatgtttcagaaacaactctggcgcatcgggcttcccata |
| | | caagcgatagattgtcgcacctgattgcccgacattatcgcgagccca |
| | | tttatacccatataaatcagcatccatgttggaatttaatcgcggcct |
| | | cgacgtttcccgttgaatatggctcatactcttcctttttcaatatta |
| | | ttgaagcatttatcagggttattgtctcatgagcggatacatatttga |
| | | atgtatttagaaaaataaacaaataggggttccgcgcacatttccccg |
| | | aaaagtgccacctgacgtctaagaaaccattattatcatgacattaac |
| | | ctataaaaataggcgtatcacgaggccctttcgtc |
| pAAV2ITR- | 7 | tcgcgcgtttcggtgatgacggtgaaaacctctgacacatgcagctcc |
| CAG- | | cggagacggtcacagcttgtctgtaagcggatgccgggagcagacaag |
| hGALT | | cccgtcagggcgcgtcagcgggtgttggcgggtgtcggggctggctta |
| plasmid | | actatgcggcatcagagcagattgtactgagagtgcaccatatgcggt |
| | | gtgaaataccgcacagatgcgtaaggagaaaataccgcatcaggcgcc |
| | | attcgccattcaggctgcgcaactgttgggaagggcgatcggtgcggg |
| | | cctcttcgctattacgccagctggcgaaagggggatgtgctgcaaggc |
| | | gattaagttgggtaacgccagggttttcccagtcacgacgttgtaaaa |
| | | cgacggccagtaggcgcgcccaaaggctatcaggtcattgcctgagag |
| | | tctggagcaaacaagagaatcgttggccactccctctctgcgcgctcg |
| | | ctcgctcactgaggccgcccgggcaaagcccgggcgtcgggcgacctt |
| | | tggtcgcccggcctcagtgagcgagcgagcgcgcagagagggagtggc |
| | | caactccatcactaggggttcctgatgtaatacgactcactagttatt |
| | | aatagtaatcaattacggggtcattagttcatagcccatatatggagt |
| | | tccgcgttacataacttacggtaaatggcccgcctggctgaccgccca |
| | | acgacccccgcccattgacgtcaataatgacgtatgttcccatagtaa |
| | | cgccaatagggactttccattgacgtcaatgggtggactatttacggt |
| | | aaactgcccacttggcagtacatcaagtgtatcatatgccaagtacgc |
| | | cccctattgacgtcaatgacggtaaatggcccgcctggcattatgccc |
| | | agtacatgaccttatgggactttcctacttggcagtacatctacgtat |
| | | tagtcatcgctattaccatgggtcgaggtgagccccacgttctgcttc |
| | | actctccccatctcccccccctccccacccccaattttgtatttattt |
| | | attttttaattattttgtgcagcgatgggggcggggggggggggggcg |
| | | cgcgccaggcggggcggggcggggcgaggggcggggcggggcgaggcg |
| | | gagaggtgcggcggcagccaatcagagcggcgcgctccgaaagtttcc |
| | | ttttatggcgaggcggcggcggcggcggccctataaaaagcgaagcgc |
| | | gcggcgggcgggagtcgctgcgttgccttcgccccgtgccccgctccg |
| | | cgccgcctcgcgccgcccgccccggctctgactgaccgcgttactccc |
| | | acaggtgagcgggcgggacggcccttctcctccgggctgtaattagcg |
| | | cttggtttaatgacggctcgtttcttttctgtggctgcgtgaaagcct |
| | | taaagggctccgggagggccctttgtgcgggggggagcggctcggggg |
| | | gtgcgtgcgtgtgtgtgtgcgtggggagcgccgcgtgcggcccgcgct |
| | | gcccggcggctgtgagcgctgcgggcgcggcgcggggctttgtgcgct |
| | | ccgcgtgtgcgcgaggggagcgcggccgggggcggtgccccgcggtgc |
| | | gggggggctgcgaggggaacaaaggctgcgtgcggggtgtgtgcgtgg |
| | | gggggtgagcagggggtgtgggcgcggcggtcgggctgtaaccccccc |
| | | ctgcacccccctccccgagttgctgagcacggcccggcttcgggtgcg |
| | | gggctccgtgcggggcgtggcgcggggctcgccgtgccgggcgggggg |
| | | tggcggcaggtgggggtgccgggcggggcggggccgcctcgggccggg |
| | | gagggctcgggggaggggcgcggcggccccggagcgccggcggctgtc |
| | | gaggcgcggcgagccgcagccattgccttttatggtaatcgtgcgaga |
| | | gggcgcagggacttcctttgtcccaaatctggcggagccgaaatctgg |
| | | gaggcgccgccgcaccccctctagcgggcgcgggcgaagcggtgcggc |
| | | gccggcaggaaggaaatgggcggggagggccttcgtgcgtcgccgcgc |
| | | cgccgtccccttctccatctccagcctcggggctgccgcagggggacg |
| | | gctgccttcgggggggacggggcagggcggggttcggcttctggcgtg |
| | | tgaccggcggctctagagcctctgctaaccatgttcatgccttcttct |
| | | ttttcctacaggccaccatgtcgcgcagtggaaccgatcctcagcaac |
| | | gccagcaggcgtcagaggcggacgccgcagcagcaaccttccgggcaa |
| | | acgaccatcagcatatccgctacaacccgctgcaggatgagtgggtgc |
| | | tggtgtcagctcaccgcatgaagcggccctggcagggtcaagtggagc |
| | | cccagcttctgaagacagtgccccgccatgaccctctcaaccctctgt |
| | | gtcctggggccatccgagccaacggagaggtgaatccccagtacgata |
| | | gcaccttcctgtttgacaacgacttcccagctctgcagcctgatgccc |
| | | ccagtccaggacccagtgatcatccccttttccaagcaaagtctgctc |
| | | gaggagtctgtaaggtcatgtgcttccacccctggtcggatgtaacgc |
| | | tgccactcatgtcggtccctgagatccgggctgttgttgatgcatggg |
| | | cctcagtcacagaggagctgggtgcccagtacccttgggtgcagatct |
| | | ttgaaaacaaaggtgccatgatgggctgttctaacccccacccccact |
| | | gccaggtatgggccagcagtttcctgccagatattgcccagcgtgagg |
| | | agcgatctcagcaggcctataagagtcagcatggagagcccctgctaa |
| | | tggagtacagccgccaggagctactcaggaaggaacgtctggtcctaa |
| | | ccagtgagcactggttagtactggtccccttctgggcaacatggccct |
| | | accagacactgctgctgccccgtcggcatgtgcggcggctacctgagc |
| | | tgacccctgctgagcgtgatgatctagcctccatcatgaagaagctct |
| | | tgaccaagtatgacaacctctttgagacgtcctttccctactccatgg |
| | | gctggcatggggctcccacaggatcagaggctggggccaactggaacc |
| | | attggcagctgcacgctcattactaccctccgctcctgcgctctgcca |
| | | ctgtccggaaattcatggttggctacgaaatgcttgctcaggctcaga |
| | | gggacctcacccctgagcaggctgcagagagactaagggcacttcctg |
| | | aggttcattaccacctggggcagaaggacagggagacagcaaccatcg |
| | | cctgacggcgcgccaagcttatcgataatcaacctctggattacaaaa |
| | | tttgtgaaagattgactggtattcttaactatgttgctccttttacgc |
| | | tatgtggatacgctgctttaatgcctttgtatcatgctattgcttccc |
| | | gtatggctttcattttctcctccttgtataaatcctggtagctgtctc |
| | | tttatgaggagttgtggcccgttgtcaggcaacgtggcgtagtgtgca |
| | | ctgtgtttgctgacgcaacccccactggttggggcattgccaccacct |
| | | gtcagctcctttccgggactttcgctttccccctccctattgccacgg |
| | | cggaactcatcgccgcctgccttgcccgctgctggacaggggctcggc |
| | | tgttgggcactgacaattccgtggtgttgtcggggaaatcatcgtcct |
| | | ttccttggctgctcgcctgtgttgccacctggattctgcgcgggacgt |
| | | ccttctgctacgtcccttcggccctcaatccagcggaccttccttccc |
| | | gcggcctgctgccggctctgcggcctcttccgcgtcttcgccttcgcc |
| | | ctcagacgagtcggatctccctttgggccgcctccccgcatcgatacc |
| | | gagcgctgctcgactgtgccttctagttgccagccatctgttgtttgc |
| | | ccctcccccgtgccttccttgaccctggaaggtgccactcccactgtc |
| | | ctttcctaataaaatgaggaaattgcatcgcattgtctgagtaggtgt |
| | | cattctattctggggggtggggtggggcaggacagcaagggggaggat |
| | | tgggaagacaatagcaggcatgctggggatgcggtgggctctatggga |
| | | gatctacaggcctaggtgagctctggtaccctctagtcaaggatcagg |
| | | aacccctagtgatggagttggccactccctctctgcgcgctcgctcgc |
| | | tcactgaggccgggcgaccaaaggtcgcccgacgcccgggctttgccc |
| | | gggcggcctcagtgagcgagcgagcgcgcagagagggagtggccaact |
| | | gcattaatgaatcggccaacgcgcggggagaggcggtttgcgtattgg |
| | | ttaattaacatggtcatagctgtttcctgtgtgaaattgttatccgct |
| | | cacaattccacacaacatacgagccggaagcataaagtgtaaagcctg |
| | | gggtgcctaatgagtgagctaactcacattaattgcgttgcgctcact |
| | | gcccgctttccagtcgggaaacctgtcgtgccagctgcattaatgaat |
| | | cggccaacgcgcggggagaggcggtttgcgtattgggcgctcttccgc |
| | | ttcctcgctcactgactcgctgcgctcggtcgttcggctgcggcgagc |
| | | ggtatcagctcactcaaaggcggtaatacggttatccacagaatcagg |
| | | ggataacgcaggaaagaacatgtgagcaaaaggccagcaaaaggccag |
| | | gaaccgtaaaaaggccgcgttgctggcgtttttccataggctccgccc |
| | | ccctgacgagcatcacaaaaatcgacgctcaagtcagaggtggcgaaa |
| | | cccgacaggactataaagataccaggcgtttccccctggaagctccct |
| | | cgtgcgctctcctgttccgaccctgccgcttaccggatacctgtccgc |
| | | ctttctcccttcgggaagcgtggcgctttctcatagctcacgctgtag |
| | | gtatctcagttcggtgtaggtcgttcgctccaagctgggctgtgtgca |
| | | cgaaccccccgttcagcccgaccgctgcgccttatccggtaactatcg |
| | | tcttgagtccaacccggtaagacacgacttatcgccactggcagcagc |
| | | cactggtaacaggattagcagagcgaggtatgtaggcggtgctacaga |
| | | gttcttgaagtggtggcctaactacggctacactagaagaacagtatt |
| | | tggtatctgcgctctgctgaagccagttaccttcggaaaaagagttgg |
| | | tagctcttgatccggcaaacaaaccaccgctggtagcggtggtttttt |
| | | tgtttgcaagcagcagattacgcgcagaaaaaaaggatctcaagaaga |
| | | tcctttgatcttttctacggggtctgacgctcagtggaacgaaaactc |
| | | acgttaagggattttggtcatgagattatcaaaaaggatcttcaccta |
| | | gatccttttaaattaaaaatgaagttttaaatcaatctaaagtatata |
| | | tgagtaaacttggtctgacagttagaaaaactcatcgagcatcaaatg |
| | | aaactgcaatttattcatatcaggattatcaataccatatttttgaaa |
| | | aagccgtttctgtaatgaaggagaaaactcaccgaggcagttccatag |
| | | gatggcaagatcctggtatcggtctgcgattccgactcgtccaacatc |
| | | aatacaacctattaatttcccctcgtcaaaaataaggttatcaagtga |
| | | gaaatcaccatgagtgacgactgaatccggtgagaatggcaaaagttt |
| | | atgcatttctttccagacttgttcaacaggccagccattacgctcgtc |
| | | atcaaaatcactcgcatcaaccaaaccgttattcattcgtgattgcgc |
| | | ctgagcgagacgaaatacgcgatcgctgttaaaaggacaattacaaac |
| | | aggaatcgaatgcaaccggcgcaggaacactgccagcgcatcaacaat |
| | | attttcacctgaatcaggatattcttctaatacctggaatgctgtttt |
| | | tccggggatcgcagtggtgagtaaccatgcatcatcaggagtacggat |
| | | aaaatgcttgatggtcggaagaggcataaattccgtcagccagtttag |
| | | tctgaccatctcatctgtaacatcattggcaacgctacctttgccatg |
| | | tttcagaaacaactctggcgcatcgggcttcccatacaagcgatagat |
| | | tgtcgcacctgattgcccgacattatcgcgagcccatttatacccata |
| | | taaatcagcatccatgttggaatttaatcgcggcctcgacgtttcccg |
| | | ttgaatatggctcatactcttcctttttcaatattattgaagcattta |
| | | tcagggttattgtctcatgagcggatacatatttgaatgtatttagaa |
| | | aaataaacaaataggggttccgcgcacatttccccgaaaagtgccacc |
| | | tgacgtctaagaaaccattattatcatgacattaacctataaaaatag |
| | | gcgtatcacgaggccctttcgtc |
| pAAV2ITR- | 8 | tcgcgcgtttcggtgatgacggtgaaaacctctgacacatgcagctcc |
| EF1a- | | cggagacggtcacagcttgtctgtaagcggatgccgggagcagacaag |
| hGALT | | cccgtcagggcgcgtcagcgggtgttggcgggtgtcggggctggctta |
| plasmid | | actatgcggcatcagagcagattgtactgagagtgcaccatatgcggt |
| | | gtgaaataccgcacagatgcgtaaggagaaaataccgcatcaggcgcc |
| | | attcgccattcaggctgcgcaactgttgggaagggcgatcggtgcggg |
| | | cctcttcgctattacgccagctggcgaaagggggatgtgctgcaaggc |
| | | gattaagttgggtaacgccagggttttcccagtcacgacgttgtaaaa |
| | | cgacggccagtaggcgcgcccaaaggctatcaggtcattgcctgagag |
| | | tctggagcaaacaagagaatcgttggccactccctctctgcgcgctcg |
| | | ctcgctcactgaggccgcccgggcaaagcccgggcgtcgggcgacctt |
| | | tggtcgcccggcctcagtgagcgagcgagcgcgcagagagggagtggc |
| | | caactccatcactaggggttcctgatgtaatacgactcactagtgctc |
| | | cggtgcccgtcagtgggcagagcgcacatcgcccacagtccccgagaa |
| | | gttggggggaggggtcggcaattgaaccggtgcctagagaaggtggcg |
| | | cggggtaaactgggaaagtgatgtcgtgtactggctccgcctttttcc |
| | | cgagggtgggggagaaccgtatataagtgcagtagtcgccgtgaacgt |
| | | tctttttcgcaacgggtttgccgccagaacacaggtaagtgccgtgtg |
| | | tggttcccgcgggcctggcctctttacgggttatggcccttgcgtgcc |
| | | ttgaattacttccacgcccctggctgcagtacgtgattcttgatcccg |
| | | agcttcgggttggaagtgggtgggagagttcgaggccttgcgcttaag |
| | | gagccccttcgcctcgtgcttgagttgaggcctggcctgggcgctggg |
| | | gccgccgcgtgcgaatctggtggcaccttcgcgcctgtctcgctgctt |
| | | tcgataagtctctagccatttaaaatttttgatgacctgctgcgacgc |
| | | tttttttctggcaagatagtcttgtaaatgcgggccaagatctgcaca |
| | | ctggtatttcggtttttggggccgcgggcggcgacggggcccgtgcgt |
| | | cccagcgcacatgttcggcgaggcggggcctgcgagcgcggccaccga |
| | | gaatcggacgggggtagtctcaagctggccggcctgctctggtgcctg |
| | | gcctcgcgccgccgtgtatcgccccgccctgggcggcaaggctggccc |
| | | ggtcggcaccagttgcgtgagcggaaagatggccgcttcccggccctg |
| | | ctgcagggagctcaaaatggaggacgcggcgctcgggagagcgggcgg |
| | | gtgagtcacccacacaaaggaaaagggcctttccgtcctcagccgtcg |
| | | cttcatgtgactccacggagtaccgggcgccgtccaggcacctcgatt |
| | | agttctcgagcttttggagtacgtcgtctttaggttggggggaggggt |
| | | tttatgcgatggagtttccccacactgagtgggtggagactgaagtta |
| | | ggccagcttggcacttgatgtaattctccttggaatttgccctttttg |
| | | agtttggatcttggttcattctcaagcctcagacagtggttcaaagtt |
| | | tttttcttccatttcaggtgtcgtgagccaccatgtcgcgcagtggaa |
| | | ccgatcctcagcaacgccagcaggcgtcagaggcggacgccgcagcag |
| | | caaccttccgggcaaacgaccatcagcatatccgctacaacccgctgc |
| | | aggatgagtgggtgctggtgtcagctcaccgcatgaagcggccctggc |
| | | agggtcaagtggagccccagcttctgaagacagtgccccgccatgacc |
| | | ctctcaaccctctgtgtcctggggccatccgagccaacggagaggtga |
| | | atccccagtacgatagcaccttcctgtttgacaacgacttcccagctc |
| | | tgcagcctgatgcccccagtccaggacccagtgatcatccccttttcc |
| | | aagcaaagtctgctcgaggagtctgtaaggtcatgtgcttccacccct |
| | | ggtcggatgtaacgctgccactcatgtcggtccctgagatccgggctg |
| | | ttgttgatgcatgggcctcagtcacagaggagctgggtgcccagtacc |
| | | cttgggtgcagatctttgaaaacaaaggtgccatgatgggctgttcta |
| | | acccccacccccactgccaggtatgggccagcagtttcctgccagata |
| | | ttgcccagcgtgaggagcgatctcagcaggcctataagagtcagcatg |
| | | gagagcccctgctaatggagtacagccgccaggagctactcaggaagg |
| | | aacgtctggtcctaaccagtgagcactggttagtactggtccccttct |
| | | gggcaacatggccctaccagacactgctgctgccccgtcggcatgtgc |
| | | ggcggctacctgagctgacccctgctgagcgtgatgatctagcctcca |
| | | tcatgaagaagctcttgaccaagtatgacaacctctttgagacgtcct |
| | | ttccctactccatgggctggcatggggctcccacaggatcagaggctg |
| | | gggccaactggaaccattggcagctgcacgctcattactaccctccgc |
| | | tcctgcgctctgccactgtccggaaattcatggttggctacgaaatgc |
| | | ttgctcaggctcagagggacctcacccctgagcaggctgcagagagac |
| | | taagggcacttcctgaggttcattaccacctggggcagaaggacaggg |
| | | agacagcaaccatcgcctgacggcgcgccaagcttatcgataatcaac |
| | | ctctggattacaaaatttgtgaaagattgactggtattcttaactatg |
| | | ttgctccttttacgctatgtggatacgctgctttaatgcctttgtatc |
| | | atgctattgcttcccgtatggctttcattttctcctccttgtataaat |
| | | cctggtagctgtctctttatgaggagttgtggcccgttgtcaggcaac |
| | | gtggcgtagtgtgcactgtgtttgctgacgcaacccccactggttggg |
| | | gcattgccaccacctgtcagctcctttccgggactttcgctttccccc |
| | | tccctattgccacggcggaactcatcgccgcctgccttgcccgctgct |
| | | ggacaggggctcggctgttgggcactgacaattccgtggtgttgtcgg |
| | | ggaaatcatcgtcctttccttggctgctcgcctgtgttgccacctgga |
| | | ttctgcgcgggacgtccttctgctacgtcccttcggccctcaatccag |
| | | cggaccttccttcccgcggcctgctgccggctctgcggcctcttccgc |
| | | gtcttcgccttcgccctcagacgagtcggatctccctttgggccgcct |
| | | ccccgcatcgataccgagcgctgctcgagagatctacctgtgccttct |
| | | agttgccagccatctgttgtttgcccctcccccgtgccttccttgacc |
| | | ctggaaggtgccactcccactgtcctttcctaataaaatgaggaaatt |
| | | gcatcgcattgtctgagtaggtgtcattctattctggggggtggggtg |
| | | gggcaggacagcaagggggaggattgggaagacaatagcaggcatgct |
| | | ggggatgcggtgggctctatggaggcctaggtgagctctggtaccctc |
| | | tagtcaaggatcaggaacccctagtgatggagttggccactccctctc |
| | | tgcgcgctcgctcgctcactgaggccgggcgaccaaaggtcgcccgac |
| | | gcccgggctttgcccgggcggcctcagtgagcgagcgagcgcgcagag |
| | | agggagtggccaactgcattaatgaatcggccaacgcgcggggagagg |
| | | cggtttgcgtattggttaattaacatggtcatagctgtttcctgtgtg |
| | | aaattgttatccgctcacaattccacacaacatacgagccggaagcat |
| | | aaagtgtaaagcctggggtgcctaatgagtgagctaactcacattaat |
| | | tgcgttgcgctcactgcccgctttccagtcgggaaacctgtcgtgcca |
| | | gctgcattaatgaatcggccaacgcgcggggagaggcggtttgcgtat |
| | | tgggcgctcttccgcttcctcgctcactgactcgctgcgctcggtcgt |
| | | tcggctgcggcgagcggtatcagctcactcaaaggcggtaatacggtt |
| | | atccacagaatcaggggataacgcaggaaagaacatgtgagcaaaagg |
| | | ccagcaaaaggccaggaaccgtaaaaaggccgcgttgctggcgttttt |
| | | ccataggctccgcccccctgacgagcatcacaaaaatcgacgctcaag |
| | | tcagaggtggcgaaacccgacaggactataaagataccaggcgtttcc |
| | | ccctggaagctccctcgtgcgctctcctgttccgaccctgccgcttac |
| | | cggatacctgtccgcctttctcccttcgggaagcgtggcgctttctca |
| | | tagctcacgctgtaggtatctcagttcggtgtaggtcgttcgctccaa |
| | | gctgggctgtgtgcacgaaccccccgttcagcccgaccgctgcgcctt |
| | | atccggtaactatcgtcttgagtccaacccggtaagacacgacttatc |
| | | gccactggcagcagccactggtaacaggattagcagagcgaggtatgt |
| | | aggcggtgctacagagttcttgaagtggtggcctaactacggctacac |
| | | tagaagaacagtatttggtatctgcgctctgctgaagccagttacctt |
| | | cggaaaaagagttggtagctcttgatccggcaaacaaaccaccgctgg |
| | | tagcggtggtttttttgtttgcaagcagcagattacgcgcagaaaaaa |
| | | aggatctcaagaagatcctttgatcttttctacggggtctgacgctca |
| | | gtggaacgaaaactcacgttaagggattttggtcatgagattatcaaa |
| | | aaggatcttcacctagatccttttaaattaaaaatgaagttttaaatc |
| | | aatctaaagtatatatgagtaaacttggtctgacagttagaaaaactc |
| | | atcgagcatcaaatgaaactgcaatttattcatatcaggattatcaat |
| | | accatatttttgaaaaagccgtttctgtaatgaaggagaaaactcacc |
| | | gaggcagttccataggatggcaagatcctggtatcggtctgcgattcc |
| | | gactcgtccaacatcaatacaacctattaatttcccctcgtcaaaaat |
| | | aaggttatcaagtgagaaatcaccatgagtgacgactgaatccggtga |
| | | gaatggcaaaagtttatgcatttctttccagacttgttcaacaggcca |
| | | gccattacgctcgtcatcaaaatcactcgcatcaaccaaaccgttatt |
| | | cattcgtgattgcgcctgagcgagacgaaatacgcgatcgctgttaaa |
| | | aggacaattacaaacaggaatcgaatgcaaccggcgcaggaacactgc |
| | | cagcgcatcaacaatattttcacctgaatcaggatattcttctaatac |
| | | ctggaatgctgtttttccggggatcgcagtggtgagtaaccatgcatc |
| | | atcaggagtacggataaaatgcttgatggtcggaagaggcataaattc |
| | | cgtcagccagtttagtctgaccatctcatctgtaacatcattggcaac |
| | | gctacctttgccatgtttcagaaacaactctggcgcatcgggcttccc |
| | | atacaagcgatagattgtcgcacctgattgcccgacattatcgcgagc |
| | | ccatttatacccatataaatcagcatccatgttggaatttaatcgcgg |
| | | cctcgacgtttcccgttgaatatggctcatactcttcctttttcaata |
| | | ttattgaagcatttatcagggttattgtctcatgagcggatacatatt |
| | | tgaatgtatttagaaaaataaacaaataggggttccgcgcacatttcc |
| | | ccgaaaagtgccacctgacgtctaagaaaccattattatcatgacatt |
| | | aacctataaaaataggcgtatcacgaggccctttcgtc |
| CAG | 9 | ccattgacgtcaataatgacgtatgttcccatagtaacgccaataggg |
| promoter | | actttccattgacgtcaatgggtggagtatttacggtaaactgcccac |
| | | ttggcagtacatcaagtgtatcatatgccaagtacgccccctattgac |
| | | gtcaatgacggtaaatggcccgcctggcattatgcccagtacatgacc |
| | | ttatgggactttcctacttggcagtacatctacgtattagtcatcgct |
| | | attaccatggtcgaggtgagccccacgttctgcttcactctccccatc |
| | | tcccccccctccccacccccaattttgtatttatttattttttaatta |
| | | ttttgtgcagcgatgggggcggggggggggggggggcgcgcgccaggc |
| | | ggggcggggcggggcgaggggcggggcggggcgaggcggagaggtgcg |
| | | gcggcagccaatcagagcggcgcgctccgaaagtttccttttatggcg |
| | | aggcggcggcggcggcggccctataaaaagcgaagcgcgcggcgggcg |
| | | ggagtcgctgcgcgctgccttcgccccgtgccccgctccgccgccgcc |
| | | tcgcgccgcccgccccggctctgactgaccgcgttactcccacaggtg |
| | | agcgggcgggacggcccttctcctccgggctgtaattagcgcttgg |
| EF1alpha | 10 | gctccggtgcccgtcagtgggcagagcgcacatcgcccacagtccccg |
| promoter | | agaagttggggggaggggtcggcaattgaaccggtgcctagagaaggt |
| (including | | ggcgcggggtaaactgggaaagtgatgtcgtgtactggctccgccttt |
| Intron A) | | ttcccgagggtgggggagaaccgtatataagtgcagtagtcgccgtga |
| | | acgttctttttcgcaacgggtttgccgccagaacacaggtaagtgccg |
| | | tgtgtggttcccgcgggcctggcctctttacgggttatggcccttgcg |
| | | tgccttgaattacttccacgcccctggctgcagtacgtgattcttgat |
| | | cccgagcttcgggttggaagtgggtgggagagttcgaggccttgcgct |
| | | taaggagccccttcgcctcgtgcttgagttgaggcctggcctgggcgc |
| | | tggggccgccgcgtgcgaatctggtggcaccttcgcgcctgtctcgct |
| | | gctttcgataagtctctagccatttaaaatttttgatgacctgctgcg |
| | | acgctttttttctggcaagatagtcttgtaaatgcgggccaagatctg |
| | | cacactggtatttcggtttttggggccgcgggcggcgacggggcccgt |
| | | gcgtcccagcgcacatgttcggcgaggcggggcctgcgagcgcggcca |
| | | ccgagaatcggacgggggtagtctcaagctggccggcctgctctggtg |
| | | cctggcctcgcgccgccgtgtatcgccccgccctgggcggcaaggctg |
| | | gcccggtcggcaccagttgcgtgagcggaaagatggccgcttcccggc |
| | | cctgctgcagggagctcaaaatggaggacgcggcgctcgggagagcgg |
| | | gcgggtgagtcacccacacaaaggaaaagggcctttccgtcctcagcc |
| | | gtcgcttcatgtgactccacggagtaccgggcgccgtccaggcacctc |
| | | gattagttctcgagcttttggagtacgtcgtctttaggttggggggag |
| | | gggttttatgcgatggagtttccccacactgagtgggtggagactgaa |
| | | gttaggccagcttggcacttgatgtaattctccttggaatttgccctt |
| | | tttgagtttggatcttggttcattctcaagcctcagacagtggttcaa |
| | | agtttttttcttccatttcaggtgtcgtga |
| 5′ AAV2 | 11 | ttggccactccctctctgcgcgctcgctcgctcactgaggccgcccgg |
| ITR | | gcaaagcccgggcgtcgggcgacctttggtcgcccggcctcagtgagc |
| | | gagcgagcgcgcagagagggagtggccaactccatcactaggggttcc |
| | | t |
| 3′ AAV2 | 12 | aggaacccctagtgatggagttggccactccctctctgcgcgctcgct |
| ITR | | cgctcactgaggccgggcgaccaaaggtcgcccgacgcccgggctttg |
| | | cccgggcggcctcagtgagcgagcgagcgcgcagagagggagtggcca |
| | | a |
| Self- | 13 | ccactccctctctgcgcgctcgctcgctcactgaggccgggcgaccaa |
| complementary | | aggtcgcccgacgcccgggctttgcccgggcggcctcagtgagcgagc |
| ITR | | gagcgcgcag |
| WPRE | 14 | aatcaacctctggattacaaaatttgtgaaagattgactggtattctt |
| element | | aactatgttgctccttttacgctatgtggatacgctgctttaatgcct |
| | | ttgtatcatgctattgcttcccgtatggctttcattttctcctccttg |
| | | tataaatcctggtagctgtctctttatgaggagttgtggcccgttgtc |
| | | aggcaacgtggcgtagtgtgcactgtgtttgctgacgcaacccccact |
| | | ggttggggcattgccaccacctgtcagctcctttccgggactttcgct |
| | | ttccccctccctattgccacggcggaactcatcgccgcctgccttgcc |
| | | cgctgctggacaggggctcggctgttgggcactgacaattccgtggtg |
| | | ttgtcggggaaatcatcgtcctttccttggctgctcgcctgtgttgcc |
| | | acctggattctgcgcgggacgtccttctgctacgtcccttcggccctc |
| | | aatccagcggaccttccttcccgcggcctgctgccggctctgcggcct |
| | | cttccgcgtcttcgccttcgccctcagacgagtcggatctccctttgg |
| | | gccgcctccccgc |
| bGH polyA | 15 | ctgtgccttctagttgccagccatctgttgtttgcccctcccccgtgc |
| signal | | cttccttgaccctggaaggtgccactcccactgtcctttcctaataaa |
| | | atgaggaaattgcatcgcattgtctgagtaggtgtcattctattctgg |
| | | ggggtggggtggggcaggacagcaagggggaggattgggaagacaata |
| | | gcaggcatgctggggatgcggtgggctctatgg |
| SV40 polyA | 16 | aacttgtttattgcagcttataatggttacaaataaagcaatagcatc |
| signal | | acaaatttcacaaataaagcatttttttcactgcattctagttgtggt |
| | | ttgtccaaactcatcaatgtatctta |
| Chimeric | 17 | ggagtcgctgcgttgccttcgccccgtgccccgctccgcgccgcctcg |
| intron | | cgccgcccgccccggctctgactgaccgcgttactcccacaggtgagc |
| | | gggcgggacggcccttctcctccgggctgtaattagcgcttggtttaa |
| | | tgacggctcgtttcttttctgtggctgcgtgaaagccttaaagggctc |
| | | cgggagggccctttgtgcgggggggagcggctcggggggtgcgtgcgt |
| | | gtgtgtgtgcgtggggagcgccgcgtgcggcccgcgctgcccggcggc |
| | | tgtgagcgctgcgggcgcggcgcggggctttgtgcgctccgcgtgtgc |
| | | gcgaggggagcgcggccgggggcggtgccccgcggtgcgggggggctg |
| | | cgaggggaacaaaggctgcgtgcggggtgtgtgcgtgggggggtgagc |
| | | agggggtgtgggcgcggcggtcgggctgtaacccccccctgcaccccc |
| | | ctccccgagttgctgagcacggcccggcttcgggtgcggggctccgtg |
| | | cggggcgtggcgcggggctcgccgtgccgggcggggggtggcggcagg |
| | | tgggggtgccgggcggggcggggccgcctcgggccggggagggctcgg |
| | | gggaggggcgcggcggccccggagcgccggcggctgtcgaggcgcggc |
| | | gagccgcagccattgccttttatggtaatcgtgcgagagggcgcaggg |
| | | acttcctttgtcccaaatctggcggagccgaaatctgggaggcgccgc |
| | | cgcaccccctctagcgggcgcgggcgaagcggtgcggcgccggcagga |
| | | aggaaatgggcggggagggccttcgtgcgtcgccgcgccgccgtcccc |
| | | ttctccatctccagcctcggggctgccgcagggggacggctgccttcg |
| | | ggggggacggggcagggcggggttcggcttctggcgtgtgaccggcgg |
| | | ctctagagcctctgctaaccatgttcatgccttcttctttttcctaca |
| | | g |
| AAV9 | 18 | MAADGYLPDW LEDNLSEGIR EWWALKPGAP QPKANQQHQD |
| capsid | | NARGLVLPGY KYLGPGNGLD KGEPVNAADA AALEHDKAYD |
| | | QQLKAGDNPY LKYNHADAEF QERLKEDTSF GGNLGRAVFQ |
| | | AKKRLLEPLG LVEEAAKTAP GKKRPVEQSP QEPDSSAGIG |
| | | KSGAQPAKKR LNFGQTGDTE SVPDPQPIGE PPAAPSGVGS |
| | | LTMASGGGAP VADNNEGADG VGSSSGNWHC DSQWLGDRVI |
| | | TTSTRTWALP TYNNHLYKQI SNSTSGGSSN DNAYFGYSTP |
| | | WGYFDFNRFH CHFSPRDWQR LINNNWGFRP KRLNFKLFNI |
| | | QVKEVIDNNG VKTIANNLTS TVQVFTDSDY QLPYVLGSAH |
| | | EGCLPPFPAD VEMIPQYGYL TLNDGSQAVG RSSFYCLEYF |
| | | PSQMLRIGNN FQFSYEFENV PFHSSYAHSQ SLDRLMNPLI |
| | | DQYLYYLSKT INGSGQNQQT LKFSVAGPSN MAVQGRNYIP |
| | | GPSYRQQRVS TTVTQNNNSE FAWPGASSWA LNGRNSLMNP |
| | | GPAMASHKEG EDRFFPLSGS LIFGKQGTGR DNVDADKVMI |
| | | TNEEEIKTTN PVATESYGQV ATNHQSAQAQ AQTGWVQNQG |
| | | ILPGMVWQDR DVYLQGPIWA KIPHTDGNFH PSPLMGGFGM |
| | | KHPPPQILIK NTPVPADPPT AFNKDKLNSF ITQYSTGQVS |
| | | VEIEWELQKE NSKRWNPEIQ YTSNYYKSNN VEFAVNTEGV |
| | | YSEPRPIGTR YLTRNL |
| pscAAV- | 19 | ccattgacgtcaataatgacgtatgttcccatagtaacgccaatagggactttccatt |
| CAG- | | gacgtcaatgggtggagtatttacggtaaactgcccacttggcagtacatcaagtgta |
| hGALT | | tcatatgccaagtacgccccctattgacgtcaatgacggtaaatggcccgcctggcat |
| expression | | tatgcccagtacatgaccttatgggactttcctacttggcagtacatctacgtattag |
| cassette | | tcatcgctattaccatggtcgaggtgagccccacgttctgcttcactctccccatctc |
| (not | | ccccccctccccacccccaattttgtatttatttattttttaattattttgtgcagcg |
| including | | atgggggcggggggggggggggggcgcgcgccaggcggggcggggcggggcgaggggc |
| flanking | | ggggcggggcgaggcggagaggtgcggcggcagccaatcagagcggcgcgctccgaaa |
| ITRs) | | gtttccttttatggcgaggcggcggcggcggcggccctataaaaagcgaagcgcgcgg |
| | | cgggcgggagtcgctgcgcgctgccttcgccccgtgccccgctccgccgccgcctcgc |
| | | gccgcccgccccggctctgactgaccgcgttactcccacaggtgagcgggcgggacgg |
| | | cccttctcctccgggctgtaattagcgcttgggaattcgccaccatgagcagaagcgg |
| | | caccgaccctcagcagagacagcaggcctctgaagccgatgccgccgctgccaccttc |
| | | agagccaatgaccaccagcacatccggtacaaccccctgcaggacgagtgggtgctgg |
| | | tgtccgcccacagaatgaagaggccttggcagggccaggtggaaccccagctgctgaa |
| | | aaccgtgcccagacacgaccccctgaaccctctgtgtcctggcgccattagagccaac |
| | | ggcgaagtgaacccccagtacgacagcaccttcctgttcgacaacgacttccccgccc |
| | | tgcagcctgatgccccatctcctggacctagcgaccaccctctgttccaggccaagtc |
| | | tgccagaggcgtgtgcaaagtgatgtgcttccacccttggagcgacgtgaccctgccc |
| | | ctgatgagcgtgccagagatcagagccgtggtggatgcctgggccagcgtgacagaag |
| | | aactgggagcccagtacccctgggtgcagatcttcgagaacaagggcgccatgatggg |
| | | ctgcagcaacccccaccctcactgtcaagtgtgggccagcagcttcctgcccgatatc |
| | | gcccagcgggaagagagaagccagcaggcttacaagagccagcacggcgagcccctgc |
| | | tgatggaatactccagacaggaactgctgcggaaagaacggctggtgctgaccagcga |
| | | gcactggctggtgctggtgcctttttgggccacatggccctaccagaccctgctgctg |
| | | cctagaaggcacgtgcggagactgcctgagctgacacccgccgagagagatgacctgg |
| | | ccagcatcatgaagaaactgctgaccaaatacgacaacctgttcgagaccagcttccc |
| | | ctacagcatgggctggcacggcgctcctacaggatctgaggctggcgccaactggaac |
| | | cactggcagctgcacgcccactactaccccccactgctgagatctgccaccgtgcgga |
| | | agttcatggtgggatacgagatgctggctcaggcccagagagatctgacccctgaaca |
| | | ggccgccgaacggctgagagcactgcccgaagtgcactaccacctgggacagaaggac |
| | | agagagacagccacaatcgcctgaAGCGCTGCTCGAGAGATCTACAACTTGTTTATTG |
| | | CAGCTTATAATGGTTACAAATAAAGCAATAGCATCACAAATTTCACAAATAAAGCATT |
| | | TTTTTCACTGCATTCTAGTTGTGGTTTGTCCAAACTCATCAATGTATCTTA |
| pAAV2ITR- | 20 | CGTTACATAACTTACGGTAAATGGCCCGCCTGGCTGACCGCCCAACGACCCCCGCCCA |
| CAG- | | TTGACGTCAATAATGACGTATGTTCCCATAGTAACGCCAATAGGGACTTTCCATTGAC |
| hGALT | | GTCAATGGGTGGACTATTTACGGTAAACTGCCCACTTGGCAGTACATCAAGTGTATCA |
| expression | | TATGCCAAGTACGCCCCCTATTGACGTCAATGACGGTAAATGGCCCGCCTGGCATTAT |
| cassette | | GCCCAGTACATGACCTTATGGGACTTTCCTACTTGGCAGTACATCTACGTATTAGTCA |
| (not | | TCGCTATTACCATGGGTCGAGGTGAGCCCCACGTTCTGCTTCACTCTCCCCATCTCCC |
| including | | CCCCCTCCCCACCCCCAATTTTGTATTTATTTATTTTTTAATTATTTTGTGCAGCGAT |
| flanking | | GGGGGCGGGGGGGGGGGGGGCGCGCGCCAGGCGGGGGGGGGGGGGGCGAGGGGCGGGG |
| ITRs) | | CGGGGCGAGGCGGAGAGGTGCGGCGGCAGCCAATCAGAGCGGCGCGCTCCGAAAGTTT |
| | | CCTITTATGGCGAGGCGGCGGCGGCGGCGGCCCTATAAAAAGCGAAGCGCGCGGCGGG |
| | | CGGGAGTCGCTGCGTTGCCTTCGCCCCGTGCCCCGCTCCGCGCCGCCTCGCGCCGCCC |
| | | GCCCCGGCTCTGACTGACCGCGTTACTCCCACAGGTGAGCGGGCGGGACGGCCCTTCT |
| | | CCTCCGGGCTGTAATTAGCGCTTGGTTTAATGACGGCTCGTTTCTTTTCTGTGGCTGC |
| | | GTGAAAGCCTTAAAGGGCTCCGGGAGGGCCCTTTGTGCGGGGGGGAGCGGCTCGGGGG |
| | | GTGCGTGCGTGTGTGTGTGCGTGGGGAGCGCCGCGTGCGGCCCGCGCTGCCCGGCGGC |
| | | TGTGAGCGCTGCGGGCGCGGCGCGGGGCTTTGTGCGCTCCGCGTGTGCGCGAGGGGAG |
| | | CGCGGCCGGGGGCGGTGCCCCGCGGTGCGGGGGGGCTGCGAGGGGAACAAAGGCTGCG |
| | | TGCGGGGTGTGTGCGTGGGGGGGTGAGCAGGGGGTGTGGGCGCGGCGGTCGGGCTGTA |
| | | ACCCCCCCCTGCACCCCCCTCCCCGAGTTGCTGAGCACGGCCCGGCTTCGGGTGCGGG |
| | | GCTCCGTGCGGGGCGTGGCGCGGGGCTCGCCGTGCCGGGCGGGGGGTGGCGGCAGGTG |
| | | GGGGTGCCGGGCGGGGGGGGGCCGCCTCGGGCCGGGGAGGGCTCGGGGGAGGGGCGCG |
| | | GCGGCCCCGGAGCGCCGGCGGCTGTCGAGGCGCGGCGAGCCGCAGCCATTGCCTTTTA |
| | | TGGTAATCGTGCGAGAGGGCGCAGGGACTTCCTTTGTCCCAAATCTGGCGGAGCCGAA |
| | | ATCTGGGAGGCGCCGCCGCACCCCCTCTAGCGGGCGCGGGCGAAGCGGTGCGGCGCCG |
| | | GCAGGAAGGAAATGGGGGGGGAGGGCCTTCGTGCGTCGCCGCGCCGCCGTCCCCTTCT |
| | | CCATCTCCAGCCTCGGGGCTGCCGCAGGGGGACGGCTGCCTTCGGGGGGGACGGGGCA |
| | | GGGCGGGGTTCGGCTTCTGGCGTGTGACCGGCGGCTCTAGAGCCTCTGCTAACCATGT |
| | | TCATGCCTTCTTCTTTTTCCTACAGGCCACCATGTCGCGCAGTGGAACCGATCCTCAG |
| | | CAACGCCAGCAGGCGTCAGAGGCGGACGCCGCAGCAGCAACCTTCCGGGCAAACGACC |
| | | ATCAGCATATCCGCTACAACCCGCTGCAGGATGAGTGGGTGCTGGTGTCAGCTCACCG |
| | | CATGAAGCGGCCCTGGCAGGGTCAAGTGGAGCCCCAGCTTCTGAAGACAGTGCCCCGC |
| | | CATGACCCTCTCAACCCTCTGTGTCCTGGGGCCATCCGAGCCAACGGAGAGGTGAATC |
| | | CCCAGTACGATAGCACCTTCCTGTTTGACAACGACTTCCCAGCTCTGCAGCCTGATGC |
| | | CCCCAGTCCAGGACCCAGTGATCATCCCCTTTTCCAAGCAAAGTCTGCTCGAGGAGTC |
| | | TGTAAGGTCATGTGCTTCCACCCCTGGTCGGATGTAACGCTGCCACTCATGTCGGTCC |
| | | CTGAGATCCGGGCTGTTGTTGATGCATGGGCCTCAGTCACAGAGGAGCTGGGTGCCCA |
| | | GTACCCTTGGGTGCAGATCTTTGAAAACAAAGGTGCCATGATGGGCTGTTCTAACCCC |
| | | CACCCCCACTGCCAGGTATGGGCCAGCAGTTTCCTGCCAGATATTGCCCAGCGTGAGG |
| | | AGCGATCTCAGCAGGCCTATAAGAGTCAGCATGGAGAGCCCCTGCTAATGGAGTACAG |
| | | CCGCCAGGAGCTACTCAGGAAGGAACGTCTGGTCCTAACCAGTGAGCACTGGTTAGTA |
| | | CTGGTCCCCTTCTGGGCAACATGGCCCTACCAGACACTGCTGCTGCCCCGTCGGCATG |
| | | TGCGGCGGCTACCTGAGCTGACCCCTGCTGAGCGTGATGATCTAGCCTCCATCATGAA |
| | | GAAGCTCTTGACCAAGTATGACAACCTCTTTGAGACGTCCTTTCCCTACTCCATGGGC |
| | | TGGCATGGGGCTCCCACAGGATCAGAGGCTGGGGCCAACTGGAACCATTGGCAGCTGC |
| | | ACGCTCATTACTACCCTCCGCTCCTGCGCTCTGCCACTGTCCGGAAATTCATGGTTGG |
| | | CTACGAAATGCTTGCTCAGGCTCAGAGGGACCTCACCCCTGAGCAGGCTGCAGAGAGA |
| | | CTAAGGGCACTTCCTGAGGTTCATTACCACCTGGGGCAGAAGGACAGGGAGACAGCAA |
| | | CCATCGCCTGACGGCGCGCCAAGCTTATCGATAATCAACCTCTGGATTACAAAATTTG |
| | | TGAAAGATTGACTGGTATTCTTAACTATGTTGCTCCTTTTACGCTATGTGGATACGCT |
| | | GCTTTAATGCCTTTGTATCATGCTATTGCTTCCCGTATGGCTTTCATTTTCTCCTCCT |
| | | TGTATAAATCCTGGTAGCTGTCTCTTTATGAGGAGTTGTGGCCCGTTGTCAGGCAACG |
| | | TGGCGTAGTGTGCACTGTGTTTGCTGACGCAACCCCCACTGGTTGGGGCATTGCCACC |
| | | ACCTGTCAGCTCCTTTCCGGGACTTTCGCTTTCCCCCTCCCTATTGCCACGGCGGAAC |
| | | TCATCGCCGCCTGCCTTGCCCGCTGCTGGACAGGGGCTCGGCTGTTGGGCACTGACAA |
| | | TTCCGTGGTGTTGTCGGGGAAATCATCGTCCTTTCCTTGGCTGCTCGCCTGTGTTGCC |
| | | ACCTGGATTCTGCGCGGGACGTCCTTCTGCTACGTCCCTTCGGCCCTCAATCCAGCGG |
| | | ACCTTCCTTCCCGCGGCCTGCTGCCGGCTCTGCGGCCTCTTCCGCGTCTTCGCCTTCG |
| | | CCCTCAGACGAGTCGGATCTCCCTTTGGGCCGCCTCCCCGCATCGATACCGAGCGCTG |
| | | CTCGACTGTGCCTTCTAGTTGCCAGCCATCTGTTGTTTGCCCCTCCCCCGTGCCTTCC |
| | | TTGACCCTGGAAGGTGCCACTCCCACTGTCCITTCCTAATAAAATGAGGAAATTGCAT |
| | | CGCATTGTCTGAGTAGGTGTCATTCTATTCTGGGGGGTGGGGTGGGGCAGGACAGCAA |
| | | GGGGGAGGATTGGGAAGACAATAGCAGGCATGCTGGGGATGCGGTGGGCTCTATGG |
| pAAV2ITR- | 21 | gctccggtgcccgtcagtgggcagagcgcacatcgcccacagtccccgagaagttggg |
| EF1a- | | gggaggggtcggcaattgaaccggtgcctagagaaggtggcgcggggtaaactgggaa |
| hGALT | | agtgatgtcgtgtactggctccgcctttttcccgagggtgggggagaaccgtatataa |
| expression | | gtgcagtagtcgccgtgaacgttctttttcgcaacgggtttgccgccagaacacaggt |
| cassette | | aagtgccgtgtgtggttcccgcgggcctggcctctttacgggttatggcccttgcgtg |
| (not | | ccttgaattacttccacgcccctggctgcagtacgtgattcttgatcccgagcttcgg |
| including | | gttggaagtgggtgggagagttcgaggccttgcgcttaaggagccccttcgcctcgtg |
| flanking | | cttgagttgaggcctggcctgggcgctggggccgccgcgtgcgaatctggtggcacct |
| ITRs) | | tcgcgcctgtctcgctgctttcgataagtctctagccatttaaaatttttgatgacct |
| | | gctgcgacgctttttttctggcaagatagtcttgtaaatgcgggccaagatctgcaca |
| | | ctggtatttcggtttttggggccgcgggcggcgacggggcccgtgcgtcccagcgcac |
| | | atgttcggcgaggcggggcctgcgagcgcggccaccgagaatcggacgggggtagtct |
| | | caagctggccggcctgctctggtgcctggcctcgcgccgccgtgtatcgccccgccct |
| | | gggcggcaaggctggcccggtcggcaccagttgcgtgagcggaaagatggccgcttcc |
| | | cggccctgctgcagggagctcaaaatggaggacgcggcgctcgggagagcgggcgggt |
| | | gagtcacccacacaaaggaaaagggcctttccgtcctcagccgtcgcttcatgtgact |
| | | ccacggagtaccgggcgccgtccaggcacctcgattagttctcgagcttttggagtac |
| | | gtcgtctttaggttggggggaggggttttatgcgatggagtttccccacactgagtgg |
| | | gtggagactgaagttaggccagcttggcacttgatgtaattctccttggaatttgccc |
| | | tttttgagtttggatcttggttcattctcaagcctcagacagtggttcaaagtttttt |
| | | tcttccatttcaggtgtcgtgaGccaccATGTCGCGCAGTGGAACCGATCCTCAGCAA |
| | | CGCCAGCAGGCGTCAGAGGCGGACGCCGCAGCAGCAACCTTCCGGGCAAACGACCATC |
| | | AGCATATCCGCTACAACCCGCTGCAGGATGAGTGGGTGCTGGTGTCAGCTCACCGCAT |
| | | GAAGCGGCCCTGGCAGGGTCAAGTGGAGCCCCAGCTTCTGAAGACAGTGCCCCGCCAT |
| | | GACCCTCTCAACCCTCTGTGTCCTGGGGCCATCCGAGCCAACGGAGAGGTGAATCCCC |
| | | AGTACGATAGCACCTTCCTGITTGACAACGACTTCCCAGCTCTGCAGCCTGATGCCCC |
| | | CAGTCCAGGACCCAGTGATCATCCCCTTTTCCAAGCAAAGTCTGCTCGAGGAGTCTGT |
| | | AAGGTCATGTGCTTCCACCCCTGGTCGGATGTAACGCTGCCACTCATGTCGGTCCCTG |
| | | AGATCCGGGCTGTTGTTGATGCATGGGCCTCAGTCACAGAGGAGCTGGGTGCCCAGTA |
| | | CCCTTGGGTGCAGATCTTTGAAAACAAAGGTGCCATGATGGGCTGTTCTAACCCCCAC |
| | | CCCCACTGCCAGGTATGGGCCAGCAGTTTCCTGCCAGATATTGCCCAGCGTGAGGAGC |
| | | GATCTCAGCAGGCCTATAAGAGTCAGCATGGAGAGCCCCTGCTAATGGAGTACAGCCG |
| | | CCAGGAGCTACTCAGGAAGGAACGTCTGGTCCTAACCAGTGAGCACTGGTTAGTACTG |
| | | GTCCCCTTCTGGGCAACATGGCCCTACCAGACACTGCTGCTGCCCCGTCGGCATGTGC |
| | | GGCGGCTACCTGAGCTGACCCCTGCTGAGCGTGATGATCTAGCCTCCATCATGAAGAA |
| | | GCTCTTGACCAAGTATGACAACCTCTTTGAGACGTCCTTTCCCTACTCCATGGGCTGG |
| | | CATGGGGCTCCCACAGGATCAGAGGCTGGGGCCAACTGGAACCATTGGCAGCTGCACG |
| | | CTCATTACTACCCTCCGCTCCTGCGCTCTGCCACTGTCCGGAAATTCATGGTTGGCTA |
| | | CGAAATGCTTGCTCAGGCTCAGAGGGACCTCACCCCTGAGCAGGCTGCAGAGAGACTA |
| | | AGGGCACTTCCTGAGGTTCATTACCACCTGGGGCAGAAGGACAGGGAGACAGCAACCA |
| | | TCGCCTGACGGCGCGCCAAGCTTATCGATAATCAACCTCTGGATTACAAAATTTGTGA |
| | | AAGATTGACTGGTATTCTTAACTATGTTGCTCCTTTTACGCTATGTGGATACGCTGCT |
| | | TTAATGCCTTTGTATCATGCTATTGCTTCCCGTATGGCTTTCATTTTCTCCTCCTTGT |
| | | ATAAATCCTGGTAGCTGTCTCTTTATGAGGAGTTGTGGCCCGTTGTCAGGCAACGTGG |
| | | CGTAGTGTGCACTGTGTTTGCTGACGCAACCCCCACTGGTTGGGGCATTGCCACCACC |
| | | TGTCAGCTCCTTTCCGGGACTTTCGCTTTCCCCCTCCCTATTGCCACGGCGGAACTCA |
| | | TCGCCGCCTGCCTTGCCCGCTGCTGGACAGGGGCTCGGCTGTTGGGCACTGACAATTC |
| | | CGTGGTGTTGTCGGGGAAATCATCGTCCTTTCCTTGGCTGCTCGCCTGTGTTGCCACC |
| | | TGGATTCTGCGCGGGACGTCCTTCTGCTACGTCCCTTCGGCCCTCAATCCAGCGGACC |
| | | TTCCTTCCCGCGGCCTGCTGCCGGCTCTGCGGCCTCTTCCGCGTCTTCGCCTTCGCCC |
| | | TCAGACGAGTCGGATCTCCCTTTGGGCCGCCTCCCCGCATCGATACCGAGCGCTGCTC |
| | | GAGAGATCTACCTGTGCCTTCTAGTTGCCAGCCATCTGTTGTTTGCCCCTCCCCCGTG |
| | | CCTTCCTTGACCCTGGAAGGTGCCACTCCCACTGTCCTTTCCTAATAAAATGAGGAAA |
| | | TTGCATCGCATTGTCTGAGTAGGTGTCATTCTATTCTGGGGGGTGGGGTGGGGCAGGA |
| | | CAGCAAGGGGGAGGATTGGGAAGACAATAGCAGGCATGCTGGGGATGCGGTGGGCTCT |
| | | ATGG |

### REFERENCES

All publications, patent applications, patents, and other references mentioned herein (e.g., sequence database reference numbers) are incorporated by reference in their entirety. For example, all GenBank, Unigene, and Entrez sequences referred to herein, e.g., in any Table herein, are incorporated by reference. Unless otherwise specified, the sequence accession numbers specified herein, including in any Table herein, refer to the database entries current as of the filing date of this application. When one gene or protein references a plurality of sequence accession numbers, all of the sequence variants are encompassed.
US Patent Application Publication Number US 2003/0138772;
Sambrook et al, Molecular Cloning: A Laboratory Manual, Cold Spring Harbor Press, Cold Spring Harbor, N. Y.;
K. Fisher et al, J. Virol., 70:520-532 (1993);
U.S. Pat. No. 5,478,745;
U.S. Pat. No. 6,001,650;
U.S. Pat. No. 6,156,303;
Graham et al. (1973) Virology, 52:456;
Davis et al. (1986) Basic Methods in Molecular Biology, Elsevier;
Chu et al. (1981) Gene 13:197;
Wright F R, et al., Molecular Therapy (2005) 12, 171-178;
U.S. Pat. No. 5,543,158;
U.S. Pat. No. 5,641,515;
U.S. Pat. No. 5,399,363;
“Remington's Pharmaceutical Sciences” 15th Edition, pages 1035-1038 and 1570-1580;
U.S. Pat. No. 5,741,516;
U.S. Pat. No. 5,567,434;
U.S. Pat. No. 5,552,157;
U.S. Pat. No. 5,565,213;
U.S. Pat. No. 5,738,868;
U.S. Pat. No. 5,795,587;
U.S. Pat. No. 5,656,016;
U.S. Pat. No. 5,779,708;
U.S. Pat. No. 5,797,898;
U.S. Pat. No. 5,770,219;
U.S. Pat. No. 6,083,716;
U.S. Pat. No. 5,871,982;
U.S. Pat. No. 6,251,677;
U.S. Pat. No. 5,783,208; and
U.S. Pat. No. 5,697,899.

We claim:

1. A recombinant adeno-associated virus (AAV) vector comprising an expression cassette, which comprises a nucleotide sequence encoding human galactose-1-phosphate uridylyl transferase (hGALT), operably linked to one or more regulatory elements that promote expression of the hGALT coding sequence and a polyadenylation (poly(A)) tail signal; the promoter comprising a cytomegalovirus (CMV) early enhancer/chicken β-actin/rabbit β-globin splice acceptor (CAG) promoter or an elongation factor-1 (EF-1) promoter; the poly(A) tail signal comprising a bovine growth hormone (bGH) poly(A) tail signal or a simian virus 40 (SV40) poly(A) tail signal, flanked by inverted terminal repeat (ITR) nucleotide sequences.

2. The recombinant AAV vector of claim 1, the hGALT comprising the amino acid sequence of SEQ ID NO.: 1.

3. The recombinant AAV vector of claim 1 or 2, the nucleic acid that encodes GALT comprising a nucleic acid having at least 85% identity to the nucleotide sequence of SEQ ID NO.: 2 or a sequence reverse complementary thereto.

4. The recombinant AAV vector of any one of claims 1-3, the nucleic acid that encodes GALT comprising or consisting of the nucleotide sequence of SEQ ID NO.: 2 or a sequence reverse complementary thereto.

5. The recombinant AAV vector of any one of claims 1-4 wherein the promoter has a nucleotide sequence of SEQ ID NO: 9 or SEQ ID NO: 10, or the reverse complement thereof, and the polyA signal sequence has a nucleotide sequence of SEQ ID NO: 15 or SEQ ID NO: 16, or the reverse complement thereof.

6. The recombinant AAV vector of any one of claims 1-5, which comprises a WPRE element.

7. The recombinant AAV vector of any one of claims 1-6, wherein the ITRs comprise a 5′ AAV2 ITR having a nucleotide sequence of SEQ ID NO: 11 and a 3′ AAV2 ITR having a nucleotide sequence of SEQ ID NO: 12, or the reverse complement thereof.

8. The recombinant AAV vector of any one of claims 1-6 wherein the ITRs comprise a 5′ ITR having a nucleotide sequence of SEQ ID NO: 11 and a modified self-complementary 3′ ITR having a nucleotide sequence of SEQ ID NO: 13, or reverse complement thereof.

9. The recombinant AAV vector of any one of claims 1-8 comprising an expression cassette comprising a nucleic acid that has at least 85% identity to the nucleotide sequence of SEQ ID NO.: 3, SEQ ID NO.: 4, or SEQ ID NO.: 5, or a sequence reverse complementary thereto, and encodes a human GALT.

10. The recombinant AAV vector of any one of claims 1-8 comprising an expression cassette comprising a nucleic acid that has at least 85% identity to the nucleotide sequence of SEQ ID NO.: 19, SEQ ID NO.: 20, or SEQ ID NO.: 21, or a sequence reverse complementary thereto, and encodes a human GALT.

11. The recombinant AAV vector of any one of claims 1-9 comprising an expression cassette comprising a nucleic acid having a nucleotide sequence of SEQ ID NO.: 3, SEQ ID NO.: 4, or SEQ ID NO.: 5, or a sequence reverse complementary thereto.

12. The recombinant AAV vector of any one of claims 1-8 or claim 10 comprising an expression cassette comprising a nucleic acid having a nucleotide sequence of SEQ ID NO.: 19, SEQ ID NO.: 20, or SEQ ID NO.: 21, or a sequence reverse complementary thereto.

13. A recombinant self-complementary AAV (scAAV) vector comprising the recombinant AAV vector of any one of claims 1-12 comprising an scAAV ITR.

14. A recombinant AAV virion comprising: 1) an AAV capsid; and 2) the recombinant AAV vector of any one of claims 1-13; and the AAV capsid protein encapsulating the recombinant AAV vector.

15. The AAV virion of claim 14, wherein the AAV capsid has an amino acid sequence at least 85% identical to SEQ ID NO: 18 (AAV9).

16. The AAV virion of claim 15, wherein the AAV capsid has an amino acid sequence of SEQ ID NO: 18.

17. A method for treating galactosemia in a subject in need thereof, the method comprising administering to the subject a therapeutically effective amount of the AAV virion of any one of claims 14-16.

18. A method of increasing galactose metabolism in a subject in need thereof, the method comprising administering to the subject a therapeutically effective amount of the AAV virion of any one of claims 14-16.

19. A method of reducing a disease condition in a subject suffering from galactosemia, the method comprising administering to the subject a therapeutically effective amount of the AAV virion of any one of claims 14-16, the disease condition comprising jaundice, hepatosplenomegaly, hepatocellular insufficiency, hypoglycemia, renal tubular dysfunction, muscle hypotonia, sepsis, cataract, ataxia, tremor, decreased bone density, or primary ovarian insufficiency.

20. The method of any one of claims 17-19, said administering comprising intravenous administration, intra-arterial, intramuscular administration, intracardiac administration, intrathecal administration, subventricular administration, epidural administration, intracerebral administration, intracerebroventricular administration, sub-retinal administration, intravitreal administration, intraarticular administration, intraocular administration, intraperitoneal administration, intrauterine administration, intradermal administration, subcutaneous administration, transdermal administration, transmucosal administration, or administration by inhalation.

21. The method of any one of claims 17-19, the administering comprising intravenous administration, or intrathecal administration.

22. An AAV vector plasmid comprising 1) an origin of replication and 2) the recombinant AAV vector of any one of claims 1-13.

23. The AAV vector plasmid of claim 22, comprising a nucleic acid having at least 85% identity to the nucleotide sequence of SEQ ID NO.: 6, SEQ ID NO.: 7 or SEQ ID NO.: 8, which encodes a human GALT.

24. The AAV vector plasmid of claim 22, comprising a nucleic acid having at least 85% identity to the nucleotide sequence of SEQ ID NO.: 19, SEQ ID NO.: 20 or SEQ ID NO.: 21, which encodes a human GALT.

25. The AAV vector plasmid of claim 22 or 23 comprising the nucleotide sequence of SEQ ID NO.: 6, SEQ ID NO.: 7, or SEQ ID NO.: 8.

26. The AAV vector plasmid of claim 22 or 24 comprising the nucleotide sequence of SEQ ID NO.: 19, SEQ ID NO.: 20, or SEQ ID NO.: 21.

27. A cell comprising an AAV vector plasmid of any one of claims 22-26 and a second plasmid comprising nucleotide sequences encoding rep and cap; the cap encoding a VP1, a VP2, and a VP3; the rep encoding rep78, rep68, rep 52, and rep 40.

28. The cell of claim 27, the cap being AAV9 cap.

29. A method of producing an AAV virion, the method comprising culturing a host cell comprising the AAV vector plasmid of any one of claims 22-26, a second plasmid encoding the cap and rep; the cap encoding the VP1, the VP2, and the VP3; the rep encoding rep78, rep68, rep 52, and rep 40; and any additional adenoviral helper functions, under conditions sufficient to produce the AAV virion; and isolating the AAV virion produced by the host cell.


SEQUENCE LISTING (21 sequences)

SEQ ID NO: 1 - protein, 379 aa, synthetic construct
MSRSGTDPQQRQQASEADAAAATFRANDHQHIRYNPLQDEWVLVSAHRMKRPWQGQVEPQLLKTVPRHDPLNPLCPGAIRANGEVNPQYDSTFLFDNDFPALQPDAPSPGPSDHPLFQAKSARGVCKVMCFHPWSDVTLPLMSVPEIRAVVDAWASVTEELGAQYPWVQIFENKGAMMGCSNPHPHCQVWASSFLPDIAQREERSQQAYKSQHGEPLLMEYSRQELLRKERLVLTSEHWLVLVPFWATWPYQTLLLPRRHVRRLPELTPAERDDLASIMKKLLTKYDNLFETSFPYSMGWHGAPTGSEAGANWNHWQLHAHYYPPLLRSATVRKFMVGYEMLAQAQRDLTPEQAAERLRALPEVHYHLGQKDRETATIA

SEQ ID NO: 2 - DNA, 1140 bp, synthetic construct
  source 1..1140: mol_type = other DNA
atgagcagaagcggcaccgaccctcagcagagacagcaggcctctgaagccgatgccgccgctgccaccttcagagccaatgaccaccagcacatccggtacaaccccctgcaggacgagtgggtgctggtgtccgcccacagaatgaagaggccttggcagggccaggtggaaccccagctgctgaaaaccgtgcccagacacgaccccctgaaccctctgtgtcctggcgccattagagccaacggcgaagtgaacccccagtacgacagcaccttcctgttcgacaacgacttccccgccctgcagcctgatgccccatctcctggacctagcgaccaccctctgttccaggccaagtctgccagaggcgtgtgcaaagtgatgtgcttccacccttggagcgacgtgaccctgcccctgatgagcgtgccagagatcagagccgtggtggatgcctgggccagcgtgacagaagaactgggagcccagtacccctgggtgcagatcttcgagaacaagggcgccatgatgggctgcagcaacccccaccctcactgtcaagtgtgggccagcagcttcctgcccgatatcgcccagcgggaagagagaagccagcaggcttacaagagccagcacggcgagcccctgctgatggaatactccagacaggaactgctgcggaaagaacggctggtgctgaccagcgagcactggctggtgctggtgcctttttgggccacatggccctaccagaccctgctgctgcctagaaggcacgtgcggagactgcctgagctgacacccgccgagagagatgacctggccagcatcatgaagaaactgctgaccaaatacgacaacctgttcgagaccagcttcccctacagcatgggctggcacggcgctcctacaggatctgaggctggcgccaactggaaccactggcagctgcacgcccactactaccccccactgctgagatctgccaccgtgcggaagttcatggtgggatacgagatgctggctcaggcccagagagatctgacccctgaacaggccgccgaacggctgagagcactgcccgaagtgcactaccacctgggacagaaggacagagagacagccacaatcgcctga

SEQ ID NO: 3 - DNA, 2292 bp, synthetic construct
  source 1..2292: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgatgtaatacgactcactagttcctggaggggtggagtcgtgacctaggccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttgggaattcgccaccatgagcagaagcggcaccgaccctcagcagagacagcaggcctctgaagccgatgccgccgctgccaccttcagagccaatgaccaccagcacatccggtacaaccccctgcaggacgagtgggtgctggtgtccgcccacagaatgaagaggccttggcagggccaggtggaaccccagctgctgaaaaccgtgcccagacacgaccccctgaaccctctgtgtcctggcgccattagagccaacggcgaagtgaacccccagtacgacagcaccttcctgttcgacaacgacttccccgccctgcagcctgatgccccatctcctggacctagcgaccaccctctgttccaggccaagtctgccagaggcgtgtgcaaagtgatgtgcttccacccttggagcgacgtgaccctgcccctgatgagcgtgccagagatcagagccgtggtggatgcctgggccagcgtgacagaagaactgggagcccagtacccctgggtgcagatcttcgagaacaagggcgccatgatgggctgcagcaacccccaccctcactgtcaagtgtgggccagcagcttcctgcccgatatcgcccagcgggaagagagaagccagcaggcttacaagagccagcacggcgagcccctgctgatggaatactccagacaggaactgctgcggaaagaacggctggtgctgaccagcgagcactggctggtgctggtgcctttttgggccacatggccctaccagaccctgctgctgcctagaaggcacgtgcggagactgcctgagctgacacccgccgagagagatgacctggccagcatcatgaagaaactgctgaccaaatacgacaacctgttcgagaccagcttcccctacagcatgggctggcacggcgctcctacaggatctgaggctggcgccaactggaaccactggcagctgcacgcccactactaccccccactgctgagatctgccaccgtgcggaagttcatggtgggatacgagatgctggctcaggcccagagagatctgacccctgaacaggccgccgaacggctgagagcactgcccgaagtgcactaccacctgggacagaaggacagagagacagccacaatcgcctgaagcgctgctcgagagatctacaacttgtttattgcagcttataatggttacaaataaagcaatagcatcacaaatttcacaaataaagcatttttttcactgcattctagttgtggtttgtccaaactcatcaatgtatcttaaggcctaggtgagctctggtaccctctccactccctctctgcgcgctcgctcgctcactgaggccgggcgaccaaaggtcgcccgacgcccgggctttgcccgggcggcctcagtgagcgagcgagcgcgcag

SEQ ID NO: 4 - DNA, 2959 bp, synthetic construct
  source 1..2959: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgatgtaatacgactcactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacaggccaccatgtcgcgcagtggaaccgatcctcagcaacgccagcaggcgtcagaggcggacgccgcagcagcaaccttccgggcaaacgaccatcagcatatccgctacaacccgctgcaggatgagtgggtgctggtgtcagctcaccgcatgaagcggccctggcagggtcaagtggagccccagcttctgaagacagtgccccgccatgaccctctcaaccctctgtgtcctggggccatccgagccaacggagaggtgaatccccagtacgatagcaccttcctgtttgacaacgacttcccagctctgcagcctgatgcccccagtccaggacccagtgatcatccccttttccaagcaaagtctgctcgaggagtctgtaaggtcatgtgcttccacccctggtcggatgtaacgctgccactcatgtcggtccctgagatccgggctgttgttgatgcatgggcctcagtcacagaggagctgggtgcccagtacccttgggtgcagatctttgaaaacaaaggtgccatgatgggctgttctaacccccacccccactgccaggtatgggccagcagtttcctgccagatattgcccagcgtgaggagcgatctcagcaggcctataagagtcagcatggagagcccctgctaatggagtacagccgccaggagctactcaggaaggaacgtctggtcctaaccagtgagcactggttagtactggtccccttctgggcaacatggccctaccagacactgctgctgccccgtcggcatgtgcggcggctacctgagctgacccctgctgagcgtgatgatctagcctccatcatgaagaagctcttgaccaagtatgacaacctctttgagacgtcctttccctactccatgggctggcatggggctcccacaggatcagaggctggggccaactggaaccattggcagctgcacgctcattactaccctccgctcctgcgctctgccactgtccggaaattcatggttggctacgaaatgcttgctcaggctcagagggacctcacccctgagcaggctgcagagagactaagggcacttcctgaggttcattaccacctggggcagaaggacagggagacagcaaccatcgcctga

SEQ ID NO: 5 - DNA, 3543 bp, synthetic construct
  source 1..3543: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgatgtaatacgactcactagtgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagccaccatgtcgcgcagtggaaccgatcctcagcaacgccagcaggcgtcagaggcggacgccgcagcagcaaccttccgggcaaacgaccatcagcatatccgctacaacccgctgcaggatgagtgggtgctggtgtcagctcaccgcatgaagcggccctggcagggtcaagtggagccccagcttctgaagacagtgccccgccatgaccctctcaaccctctgtgtcctggggccatccgagccaacggagaggtgaatccccagtacgatagcaccttcctgtttgacaacgacttcccagctctgcagcctgatgcccccagtccaggacccagtgatcatccccttttccaagcaaagtctgctcgaggagtctgtaaggtcatgtgcttccacccctggtcggatgtaacgctgccactcatgtcggtccctgagatccgggctgttgttgatgcatgggcctcagtcacagaggagctgggtgcccagtacccttgggtgcagatctttgaaaacaaaggtgccatgatgggctgttctaacccccacccccactgccaggtatgggccagcagtttcctgccagatattgcccagcgtgaggagcgatctcagcaggcctataagagtcagcatggagagcccctgctaatggagtacagccgccaggagctactcaggaaggaacgtctggtcctaaccagtgagcactggttagtactggtccccttctgggcaacatggccctaccagacactgctgctgccccgtcggcatgtgcggcggctacctgagctgacccctgctgagcgtgatgatctagcctccatcatgaagaagctcttgaccaagtatgacaacctctttgagacgtcctttccctactccatgggctggcatggggctcccacaggatcagaggctggggccaactggaaccattggcagctgcacgctcattactaccctccgctcctgcgctctgccactgtccggaaattcatggttggctacgaaatgcttgctcaggctcagagggacctcacccctgagcaggctgcagagagactaagggcacttcctgaggttcattaccacctggggcagaaggacagggagacagcaaccatcgcctgacggcgcgccaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggtagctgtctctttatgaggagttgtggcccgttgtcaggcaacgtggcgtagtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaggcctaggtgagctctggtaccctctagtcaaggatcaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 6 - DNA, 4979 bp, synthetic construct
  source 1..4979: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtaggcgcgcccaaaggctatcaggtcattgcctgagagtctggagcaaacaagagaatcgttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgatgtaatacgactcactagttcctggaggggtggagtcgtgacctaggccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttgggaattcgccaccatgagcagaagcggcaccgaccctcagcagagacagcaggcctctgaagccgatgccgccgctgccaccttcagagccaatgaccaccagcacatccggtacaaccccctgcaggacgagtgggtgctggtgtccgcccacagaatgaagaggccttggcagggccaggtggaaccccagctgctgaaaaccgtgcccagacacgaccccctgaaccctctgtgtcctggcgccattagagccaacggcgaagtgaacccccagtacgacagcaccttcctgttcgacaacgacttccccgccctgcagcctgatgccccatctcctggacctagcgaccaccctctgttccaggccaagtctgccagaggcgtgtgcaaagtgatgtgcttccacccttggagcgacgtgaccctgcccctgatgagcgtgccagagatcagagccgtggtggatgcctgggccagcgtgacagaagaactgggagcccagtacccctgggtgcagatcttcgagaacaagggcgccatgatgggctgcagcaacccccaccctcactgtcaagtgtgggccagcagcttcctgcccgatatcgcccagcgggaagagagaagccagcaggcttacaagagccagcacggcgagcccctgctgatggaatactccagacaggaactgctgcggaaagaacggctggtgctgaccagcgagcactggctggtgctggtgcctttttgggccacatggccctaccagaccctgctgctgcctagaaggcacgtgcggagactgcctgagctgacacccgccgagagagatgacctggccagcatcatgaagaaactgctgaccaaatacgacaacctgttcgagaccagcttcccctacagcatgggctggcacggcgctcctacaggatctgaggctggcgccaactggaaccactggcagctgcacgcccactactaccccccactgctgagatctgccaccgtgcggaagttcatggtgggatacgagatgctggctcaggcccagagagatctgacccctgaacaggccgccgaacggctgagagcactgcccgaagtgcactaccacctgggacagaaggacagagagacagccacaatcgcctgaagcgctgctcgagagatctacaacttgtttattgcagcttataatggttacaaataaagcaatagcatcacaaatttcacaaataaagcatttttttcactgcattctagttgtggtttgtccaaactcatcaatgtatcttaaggcctaggtgagctctggtaccctctccactccctctctgcgcgctcgctcgctcactgaggccgggcgaccaaaggtcgcccgacgcccgggctttgcccgggcggcctcagtgagcgagcgagcgcgcagctgcattaatgaatcggccaacgcgcggggagaggcggtttgcgtattggttaattaacatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 7 - DNA, 6695 bp, synthetic construct
  source 1..6695: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtaggcgcgcccaaaggctatcaggtcattgcctgagagtctggagcaaacaagagaatcgttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgatgtaatacgactcactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacaggccaccatgtcgcgcagtggaaccgatcctcagcaacgccagcaggcgtcagaggcggacgccgcagcagcaaccttccgggcaaacgaccatcagcatatccgctacaacccgctgcaggatgagtgggtgctggtgtcagctcaccgcatgaagcggccctggcagggtcaagtggagccccagcttctgaagacagtgccccgccatgaccctctcaaccctctgtgtcctggggccatccgagccaacggagaggtgaatccccagtacgatagcaccttcctgtttgacaacgacttcccagctctgcagcctgatgcccccagtccaggacccagtgatcatccccttttccaagcaaagtctgctcgaggagtctgtaaggtcatgtgcttccacccctggtcggatgtaacgctgccactcatgtcggtccctgagatccgggctgttgttgatgcatgggcctcagtcacagaggagctgggtgcccagtacccttgggtgcagatctttgaaaacaaaggtgccatgatgggctgttctaacccccacccccactgccaggtatgggccagcagtttcctgccagatattgcccagcgtgaggagcgatctcagcaggcctataagagtcagcatggagagcccctgctaatggagtacagccgccaggagctactcaggaaggaacgtctggtcctaaccagtgagcactggttagtactggtccccttctgggcaacatggccctaccagacactgctgctgccccgtcggcatgtgcggcggctacctgagctgacccctgctgagcgtgatgatctagcctccatcatgaagaagctcttgaccaagtatgacaacctctttgagacgtcctttccctactccatgggctggcatggggctcccacaggatcagaggctggggccaactggaaccattggcagctgcacgctcattactaccctccgctcctgcgctctgccactgtccggaaattcatggttggctacgaaatgcttgctcaggctcagagggacctcacccctgagcaggctgcagagagactaagggcacttcctgaggttcattaccacctggggcagaaggacagggagacagcaaccatcgcctgacggcgcgccaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggtagctgtctctttatgaggagttgtggcccgttgtcaggcaacgtggcgtagtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggagatctacaggcctaggtgagctctggtaccctctagtcaaggatcaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaactgcattaatgaatcggccaacgcgcggggagaggcggtttgcgtattggttaattaacatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 8 - DNA, 6230 bp, synthetic construct
  source 1..6230: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtaggcgcgcccaaaggctatcaggtcattgcctgagagtctggagcaaacaagagaatcgttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgatgtaatacgactcactagtgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagccaccatgtcgcgcagtggaaccgatcctcagcaacgccagcaggcgtcagaggcggacgccgcagcagcaaccttccgggcaaacgaccatcagcatatccgctacaacccgctgcaggatgagtgggtgctggtgtcagctcaccgcatgaagcggccctggcagggtcaagtggagccccagcttctgaagacagtgccccgccatgaccctctcaaccctctgtgtcctggggccatccgagccaacggagaggtgaatccccagtacgatagcaccttcctgtttgacaacgacttcccagctctgcagcctgatgcccccagtccaggacccagtgatcatccccttttccaagcaaagtctgctcgaggagtctgtaaggtcatgtgcttccacccctggtcggatgtaacgctgccactcatgtcggtccctgagatccgggctgttgttgatgcatgggcctcagtcacagaggagctgggtgcccagtacccttgggtgcagatctttgaaaacaaaggtgccatgatgggctgttctaacccccacccccactgccaggtatgggccagcagtttcctgccagatattgcccagcgtgaggagcgatctcagcaggcctataagagtcagcatggagagcccctgctaatggagtacagccgccaggagctactcaggaaggaacgtctggtcctaaccagtgagcactggttagtactggtccccttctgggcaacatggccctaccagacactgctgctgccccgtcggcatgtgcggcggctacctgagctgacccctgctgagcgtgatgatctagcctccatcatgaagaagctcttgaccaagtatgacaacctctttgagacgtcctttccctactccatgggctggcatggggctcccacaggatcagaggctggggccaactggaaccattggcagctgcacgctcattactaccctccgctcctgcgctctgccactgtccggaaattcatggttggctacgaaatgcttgctcaggctcagagggacctcacccctgagcaggctgcagagagactaagggcacttcctgaggttcattaccacctggggcagaaggacagggagacagcaaccatcgcctgacggcgcgccaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggtagctgtctctttatgaggagttgtggcccgttgtcaggcaacgtggcgtagtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaggcctaggtgagctctggtaccctctagtcaaggatcaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaactgcattaatgaatcggccaacgcgcggggagaggcggtttgcgtattggttaattaacatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 9 - DNA, 670 bp, synthetic construct
  source 1..670: mol_type = other DNA
ccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttgg

SEQ ID NO: 10 - DNA, 1182 bp, synthetic construct
  source 1..1182: mol_type = other DNA
gctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 11 - DNA, 145 bp, synthetic construct
  source 1..145: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 12 - DNA, 145 bp, synthetic construct
  source 1..145: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 13 - DNA, 106 bp, synthetic construct
  source 1..106: mol_type = other DNA
ccactccctctctgcgcgctcgctcgctcactgaggccgggcgaccaaaggtcgcccgacgcccgggctttgcccgggcggcctcagtgagcgagcgagcgcgcag

SEQ ID NO: 14 - DNA, 589 bp, synthetic construct
  source 1..589: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggtagctgtctctttatgaggagttgtggcccgttgtcaggcaacgtggcgtagtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 15 - DNA, 225 bp, synthetic construct
  source 1..225: mol_type = other DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 16 - DNA, 122 bp, synthetic construct
  source 1..122: mol_type = other DNA
aacttgtttattgcagcttataatggttacaaataaagcaatagcatcacaaatttcacaaataaagcatttttttcactgcattctagttgtggtttgtccaaactcatcaatgtatctta

SEQ ID NO: 17 - DNA, 1009 bp, synthetic construct
  source 1..1009: mol_type = other DNA
ggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacag

SEQ ID NO: 18 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 19 - DNA, 1965 bp, synthetic construct
  source 1..1965: mol_type = other DNA
ccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttgggaattcgccaccatgagcagaagcggcaccgaccctcagcagagacagcaggcctctgaagccgatgccgccgctgccaccttcagagccaatgaccaccagcacatccggtacaaccccctgcaggacgagtgggtgctggtgtccgcccacagaatgaagaggccttggcagggccaggtggaaccccagctgctgaaaaccgtgcccagacacgaccccctgaaccctctgtgtcctggcgccattagagccaacggcgaagtgaacccccagtacgacagcaccttcctgttcgacaacgacttccccgccctgcagcctgatgccccatctcctggacctagcgaccaccctctgttccaggccaagtctgccagaggcgtgtgcaaagtgatgtgcttccacccttggagcgacgtgaccctgcccctgatgagcgtgccagagatcagagccgtggtggatgcctgggccagcgtgacagaagaactgggagcccagtacccctgggtgcagatcttcgagaacaagggcgccatgatgggctgcagcaacccccaccctcactgtcaagtgtgggccagcagcttcctgcccgatatcgcccagcgggaagagagaagccagcaggcttacaagagccagcacggcgagcccctgctgatggaatactccagacaggaactgctgcggaaagaacggctggtgctgaccagcgagcactggctggtgctggtgcctttttgggccacatggccctaccagaccctgctgctgcctagaaggcacgtgcggagactgcctgagctgacacccgccgagagagatgacctggccagcatcatgaagaaactgctgaccaaatacgacaacctgttcgagaccagcttcccctacagcatgggctggcacggcgctcctacaggatctgaggctggcgccaactggaaccactggcagctgcacgcccactactaccccccactgctgagatctgccaccgtgcggaagttcatggtgggatacgagatgctggctcaggcccagagagatctgacccctgaacaggccgccgaacggctgagagcactgcccgaagtgcactaccacctgggacagaaggacagagagacagccacaatcgcctgaagcgctgctcgagagatctacaacttgtttattgcagcttataatggttacaaataaagcaatagcatcacaaatttcacaaataaagcatttttttcactgcattctagttgtggtttgtccaaactcatcaatgtatctta

SEQ ID NO: 20 - DNA, 3594 bp, synthetic construct
  source 1..3594: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacaggccaccatgtcgcgcagtggaaccgatcctcagcaacgccagcaggcgtcagaggcggacgccgcagcagcaaccttccgggcaaacgaccatcagcatatccgctacaacccgctgcaggatgagtgggtgctggtgtcagctcaccgcatgaagcggccctggcagggtcaagtggagccccagcttctgaagacagtgccccgccatgaccctctcaaccctctgtgtcctggggccatccgagccaacggagaggtgaatccccagtacgatagcaccttcctgtttgacaacgacttcccagctctgcagcctgatgcccccagtccaggacccagtgatcatccccttttccaagcaaagtctgctcgaggagtctgtaaggtcatgtgcttccacccctggtcggatgtaacgctgccactcatgtcggtccctgagatccgggctgttgttgatgcatgggcctcagtcacagaggagctgggtgcccagtacccttgggtgcagatctttgaaaacaaaggtgccatgatgggctgttctaacccccacccccactgccaggtatgggccagcagtttcctgccagatattgcccagcgtgaggagcgatctcagcaggcctataagagtcagcatggagagcccctgctaatggagtacagccgccaggagctactcaggaaggaacgtctggtcctaaccagtgagcactggttagtactggtccccttctgggcaacatggccctaccagacactgctgctgccccgtcggcatgtgcggcggctacctgagctgacccctgctgagcgtgatgatctagcctccatcatgaagaagctcttgaccaagtatgacaacctctttgagacgtcctttccctactccatgggctggcatggggctcccacaggatcagaggctggggccaactggaaccattggcagctgcacgctcattactaccctccgctcctgcgctctgccactgtccggaaattcatggttggctacgaaatgcttgctcaggctcagagggacctcacccctgagcaggctgcagagagactaagggcacttcctgaggttcattaccacctggggcagaaggacagggagacagcaaccatcgcctgacggcgcgccaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggtagctgtctctttatgaggagttgtggcccgttgtcaggcaacgtggcgtagtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 21 - DNA, 3194 bp, synthetic construct
  source 1..3194: mol_type = other DNA
gctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagccaccatgtcgcgcagtggaaccgatcctcagcaacgccagcaggcgtcagaggcggacgccgcagcagcaaccttccgggcaaacgaccatcagcatatccgctacaacccgctgcaggatgagtgggtgctggtgtcagctcaccgcatgaagcggccctggcagggtcaagtggagccccagcttctgaagacagtgccccgccatgaccctctcaaccctctgtgtcctggggccatccgagccaacggagaggtgaatccccagtacgatagcaccttcctgtttgacaacgacttcccagctctgcagcctgatgcccccagtccaggacccagtgatcatccccttttccaagcaaagtctgctcgaggagtctgtaaggtcatgtgcttccacccctggtcggatgtaacgctgccactcatgtcggtccctgagatccgggctgttgttgatgcatgggcctcagtcacagaggagctgggtgcccagtacccttgggtgcagatctttgaaaacaaaggtgccatgatgggctgttctaacccccacccccactgccaggtatgggccagcagtttcctgccagatattgcccagcgtgaggagcgatctcagcaggcctataagagtcagcatggagagcccctgctaatggagtacagccgccaggagctactcaggaaggaacgtctggtcctaaccagtgagcactggttagtactggtccccttctgggcaacatggccctaccagacactgctgctgccccgtcggcatgtgcggcggctacctgagctgacccctgctgagcgtgatgatctagcctccatcatgaagaagctcttgaccaagtatgacaacctctttgagacgtcctttccctactccatgggctggcatggggctcccacaggatcagaggctggggccaactggaaccattggcagctgcacgctcattactaccctccgctcctgcgctctgccactgtccggaaattcatggttggctacgaaatgcttgctcaggctcagagggacctcacccctgagcaggctgcagagagactaagggcacttcctgaggttcattaccacctggggcagaaggacagggagacagcaaccatcgcctgacggcgcgccaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggtagctgtctctttatgaggagttgtggcccgttgtcaggcaacgtggcgtagtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg